(19) United States
(12) Patent Application Publication
(10) Pub. No.: US 2022/0056050 A1
(43) Pub. Date: Feb. 24, 2022

(54) COMPOUND FOR ORGANIC ELECTRONIC ELEMENT, ORGANIC ELECTRONIC ELEMENT USING THE SAME, AND AN ELECTRONIC DEVICE THEREOF

(71) Applicant: DUK SAN NEOLUX CO., LTD., Cheonan-si (KR)

(72) Inventors: Soung Yun MUN, Cheonan-si (KR); Hyung Dong LEE, Cheonan-si (KR); Sun Hee LEE, Cheonan-si (KR); Hyoung Keun PARK, Cheonan-si (KR); Dae Sic KIM, Cheonan-si (KR); Byoung Yeop KANG, Cheonan-si (KR)

(73) Assignee: DUK SAN NEOLUX CO., LTD., Cheonan-si (KR)

(21) Appl. No.: 17/445,133

(22) Filed: Aug. 16, 2021

(30) Foreign Application Priority Data
Aug. 19, 2020 (KR) 10-2020-0104018

(51) Int. Cl. C07D 519/00; H01L 51/00

(52) U.S. Cl. CPC C07D 519/00; H01L 51/5016; H01L 51/0071

(57) ABSTRACT

Provided is a novel compound capable of improving the luminous efficiency and stability of a device, an organic electronic element using the same, and an electronic device thereof.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function. And the light emitting material can be classified into a high molecular weight type and a low molecular weight type according to the molecular weight, and according to the light emission mechanism, it can be classified into a fluorescent material derived from a singlet excited state of an electron and a phosphorescent material derived from a triplet excited state of an electron. Also, the light emitting material may be divided into blue, green, and red light emitting materials and yellow and orange light emitting materials necessary for realizing a better natural color according to the emission color.

However, when only one material is used as a light emitting material, due to intermolecular interaction, the maximum emission wavelength shifts to a longer wavelength, and there are problems in that the color purity is lowered or the device efficiency is reduced due to the emission attenuation effect, therefore in order to increase color purity and increase luminous efficiency through energy transfer, a host/dopant system may be used as a light emitting material. The principle is that when a small amount of a dopant having a smaller energy band gap than that of the host forming the emitting layer is mixed in the emitting layer, excitons generated in the emitting layer are transported to the dopant to emit light with high efficiency. At this time, since the wavelength of the host moves to the wavelength band of the dopant, light having a desired wavelength can be obtained according to the type of dopant used.

Lifespan and efficiency are the most problematic in organic electroluminescent device, and as displays become larger, these problems of efficiency and lifespan must be solved. Efficiency, lifespan, and driving voltage are related to each other, and when the efficiency is increased, the driving voltage is relatively decreased, and as the driving voltage is decreased, crystallization of the organic material due to Joule heating generated during driving decreases, and as a result, the lifespan tends to increase.

However, the efficiency cannot be maximized simply by improving the organic material layer, and when the energy level and T1 value between each organic material layer, and the intrinsic properties of the material (mobility, interfacial properties, etc.) are optimally combined, a long lifespan and high efficiency can be achieved at the same time. Therefore, there is a need to develop a light emitting material that has high thermal stability and can efficiently achieve charge balance in the emitting layer. That is, in order to sufficiently exhibit the excellent characteristics of the organic electronic element, it should be preceded that materials constituting the organic material layer in the element, such as a hole injection material, a hole transport material, an emitting material, an electron transport material, an electron injection material, an emitting-auxiliary layer material, etc. are supported by a stable and efficient material, but the development of a stable and efficient organic material layer material for an organic electronic element has not yet been sufficiently made. Accordingly, the development of new materials continues to be demanded.

Recently, in addition to research on improving device characteristics by changing the performance of each material, technology for improving color purity and increasing efficiency by optimized optical thickness between the anode and cathode in a top device with a resonance structure is one of the important factors to improve performance. Compared with the bottom device structure of the non-resonant structure, the top device structure has a large optical energy loss due to surface plasmon polariton (SPP) because the formed light is reflected by the anode, which is a reflective film, and light is emitted toward the cathode.

Therefore, one of the important methods for improving the shape and efficiency of EL Spectral is a method of using a light efficiency enhancing layer for the top cathode. In general, in SPP, Al, Pt, Ag, and Au, these four metals are mainly used for electron emission, and surface plasmon is generated on the surface of the metal electrode. For example, when the cathode is used as Ag, the light emitted by the Ag of the cathode is quenched by the SPP (light energy loss due to Ag) and the efficiency is reduced.

Whereas, when the light efficiency enhancing layer is used, SPP occurs at the interface between the MgAg electrode and the high refractive organic material, and among them, transverse electric (TE) polarized light is annihilated in the vertical direction by an evanescent wave on the plane of the light efficiency enhancing layer, and TM (transverse magnetic) polarized light moving along the cathode and the light efficiency enhancing layer is amplified by surface plasma resonance, due to this, the intensity of the peak is increased, so that high efficiency and effective color purity control are possible.

### DETAILED DESCRIPTION OF THE INVENTION

### Summary

In order to solve the problems of the background art described above, the present invention has revealed a compound having a novel structure, and also found that when this compound is applied to an organic electronic element, the luminous efficiency, stability and lifespan of the element can be greatly improved.

Accordingly, an object of the present invention is to provide a novel compound, an organic electronic element using the same, and an electronic device thereof.

### Technical Solution

The present invention provides a compound represented by Formula 1.

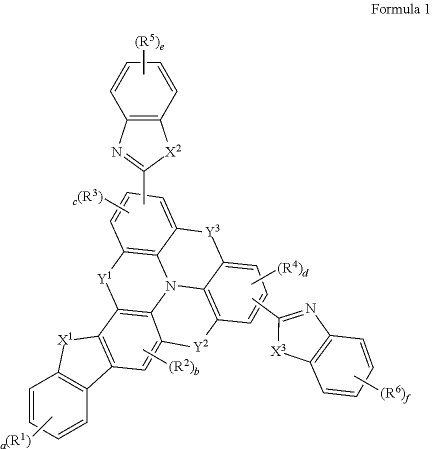

In another aspect, the present invention provides an organic electronic element comprising the compound represented by Formula 1 and an electronic device thereof.

### Effects of the Invention

By using the compound according to the present invention, high luminous efficiency of the device can be achieved, and the color purity of the device can be greatly improved.

### BRIEF DESCRIPTION OF THE DRAWINGS

FIG. 1 to FIG. 3 are exemplary views of an organic electroluminescent device according to the present invention.

FIG. 4 shows a Formula according to an aspect of the present invention.

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

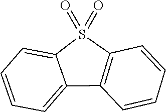 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

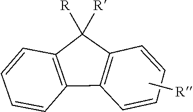

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro-’ and ‘tri-spiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

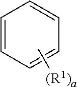

here, when a is an integer of zero, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

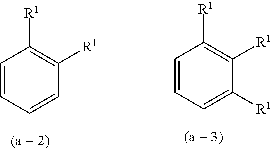

As used herein, the term “absence” means no binding unless otherwise specified.

Hereinafter, a compound according to an aspect of the present invention and an organic electronic device including the same will be described.

The present invention provides a compound represented by Formula 1.

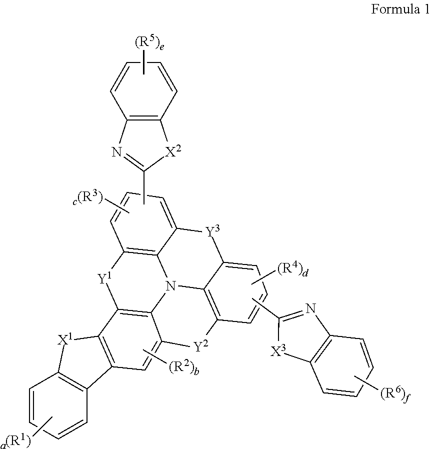

wherein, each symbol may be defined as follows.

1) X¹, X² and X³ are each independently O or S,

2) Y¹, Y² and Y³ are each independently O, S or absent.

3) R¹, R², R³, R⁴, R⁵ and R⁶ are each the same or different, and each independently selected from a group consisting of hydrogen; deuterium; halogen; cyano group; nitro group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; a C₆-C₆₀ aryloxy group; and -L′-N(R^{a})(R^{b}); or a plurality of adjacent R¹s, or a plurality of R²s, or a plurality of R³s, a plurality of adjacent R⁴s, or a plurality of R⁵s, or a plurality of R⁶s may be bonded to each other to form a ring.

R¹, R², R³, R⁴, R⁵ and R⁶ are an aryl group, they may be preferably a C₆-C₃₀ aryl group, more preferably a C₆-C₂₄ aryl group, such as phenylene, biphenyl, naphthalene, terphenyl, and the like.

R¹, R², R³, R⁴, R⁵ and R⁶ are a heterocyclic group, they may be preferably a C₂-C₃₀ heterocyclic group, and more preferably a C₂-C₂₄ heterocyclic group, for example, they may be pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.

When R¹, R², R³, R⁴, R⁵ and R⁶ are a fused ring group, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R¹, R², R³, R⁴, R⁵ and R⁶ are an alkyl group, they may be preferably an C₁˜C₃₀ alkyl group, more preferably an C₁˜C₂₄ alkyl group.

When R¹, R², R³, R⁴, R⁵ and R⁶ are an alkenyl group, they may be preferably an C₂-C₃₀ alkenyl group, more preferably an C₂-C₂₄ alkenyl group.

When R¹, R², R³, R⁴, R⁵ and R⁶ are an alkynyl group, they may be preferably a C₂-C₃₀ alkynyl group, and more preferably a C₂-C₂₄ alkynyl group.

When R¹, R², R³, R⁴, R⁵ and R⁶ are an alkoxy group, they may be preferably a C₁-C₃₀ alkoxy group, and more preferably a C₁-C₂₄ alkoxy group.

When R¹, R², R³, R⁴, R⁵ and R⁶ are an aryloxy group, they may be preferably a C₆-C₃₀ aryloxy group, and more preferably a C₆-C₂₄ aryloxy group.

4) wherein L′ is each independently selected from the group consisting of single bond; a C₆-C₆₀ arylene group; a fluorenylene group; a C₂-C₆₀ heterocyclic group including at least one heteroatom of O, N, S, Si or P; and a C₃-C₆₀ aliphatic ring; and R^{a} and R^{b} are each independently a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring;

when L′ is an arylene group, it may be preferably a C₆-C₃₀ arylene group, more preferably a C₆-C₂₄ arylene group, for example, phenylene, biphenyl, naphthalene, terphenyl, etc. when L′ is a heterocyclic group, it may be preferably a C₂-C₃₀ heterocyclic group, and more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.

when L′ is an aliphatic ring group, it may be preferably an C₃˜C₃₀ aliphatic ring, and more preferably an C₃˜C₂₄ aliphatic ring,

When R^{a} and R^{b} are an aryl group, it may be preferably a C₆-C₃₀ aryl group, and more preferably a C₆-C₂₅ aryl group, for example, phenyl, biphenyl, naphthyl, phenanthrene, terphenyl, etc.

When R^{a} and R^{b} are a heterocyclic group, it may be preferably a C₂-C₃₀ heterocyclic group, and more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazole, dibenzofuran, benzothienopyrimidine, benzofuropyrimidine, phenothiazine, phenylphenothiazine, etc.

When R^{a} and R^{b} are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

5) a, c, d, e and f are each independently an integer from 0 to 4, and b is an integer from 0 to 3.

6) wherein, the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; C₈-C₂₀ arylalkenyl group; and -L′-NR^{a}R^{b}; and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the compound represented by Formula 1 is represented by any one of Formulas 2 to 4

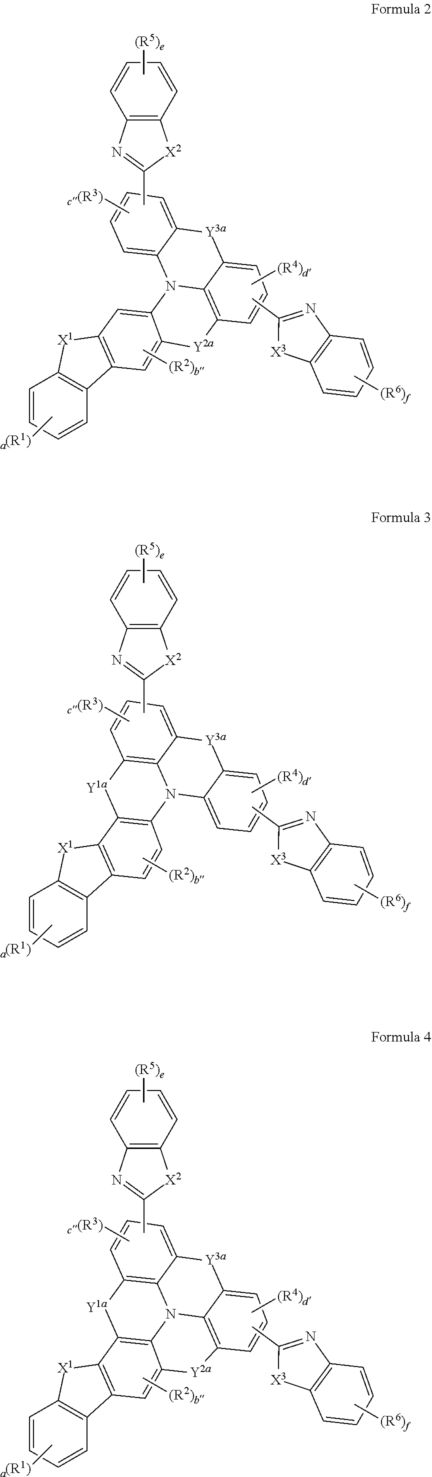

{Wherein,

1) X¹, X², X³, R¹, R², R³, R⁴, R⁵, R⁶, a, e and f are the same as defined in Formula 1,

2) Y^{1a}, Y^{2a} and Y^{3a} are each independently O or S,

3) b′ is 0 or 1, and b″, c′ and d′ are independently of each other an integer of 0 to 2, and c″ and d″ are independently of each other integers from 0 to 3.}

Also, the compound represented by Formula 1 is represented by any one of the following Formulas 5 to 7

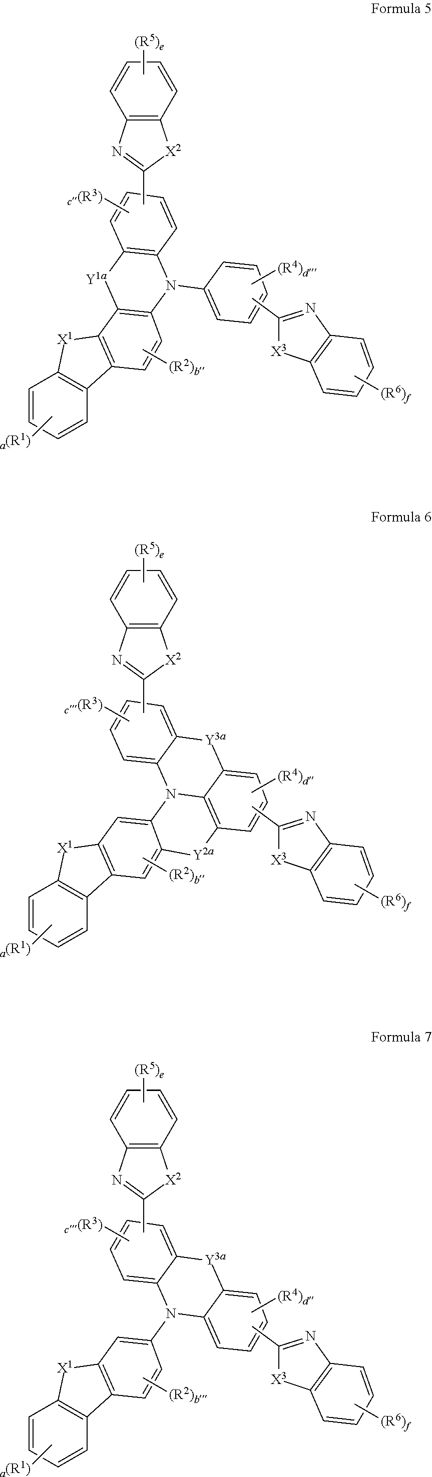

{Wherein,

1) X¹, X², X³, R¹, R², R³, R⁴, R⁵, R⁶, a, e and f are the same as defined in Formula 1,

2) Y^{1a}, Y^{2a}, Y^{3a}, b″, c″ and d″ are the same as defined in Formula 2 to Formula 4,

3) b′″ is an integer from 0 to 3, and c′″ and d′″ are independently integers from 0 to 4.}

Also, the compound represented by Formula 1 is represented by any one of the following compounds P-1 to P-84

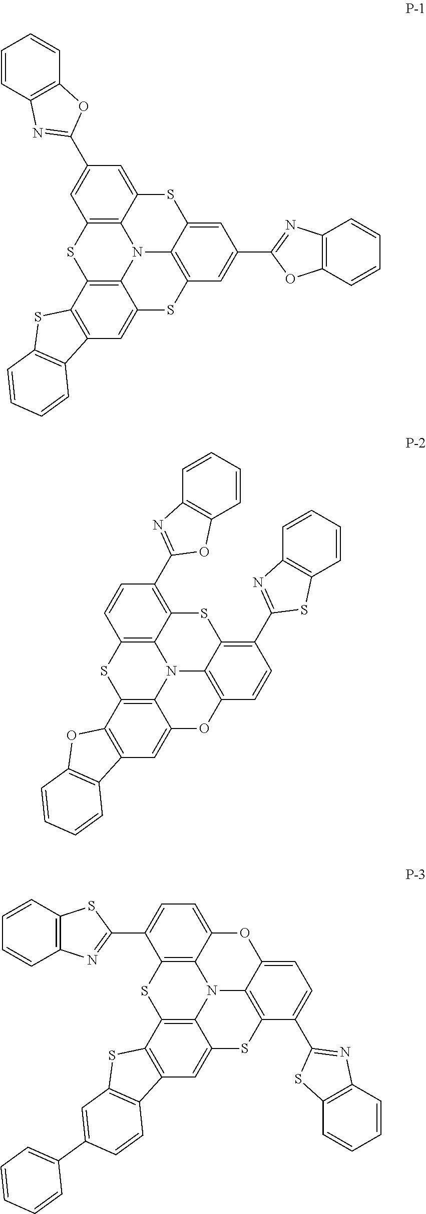 [SMILES: c1ccc2oc(-c3cc4c5c(c3)Sc3cc6c(sc7ccccc76)c6c3N5c3c(cc(-c5nc7ccccc7o5)cc3S6)S4)nc2c1 ; c1ccc2oc(-c3ccc4c5c3Sc3c(-c6nc7ccccc7s6)ccc6c3N5c3c(cc5c(oc7ccccc75)c3S4)O6)nc2c1 ; c1ccc(-c2ccc3c(c2)sc2c4c5c(cc23)Sc2c(-c3nc6ccccc6s3)ccc3c2N5c2c(ccc(-c5nc6ccccc6s5)c2S4)O3)cc1]

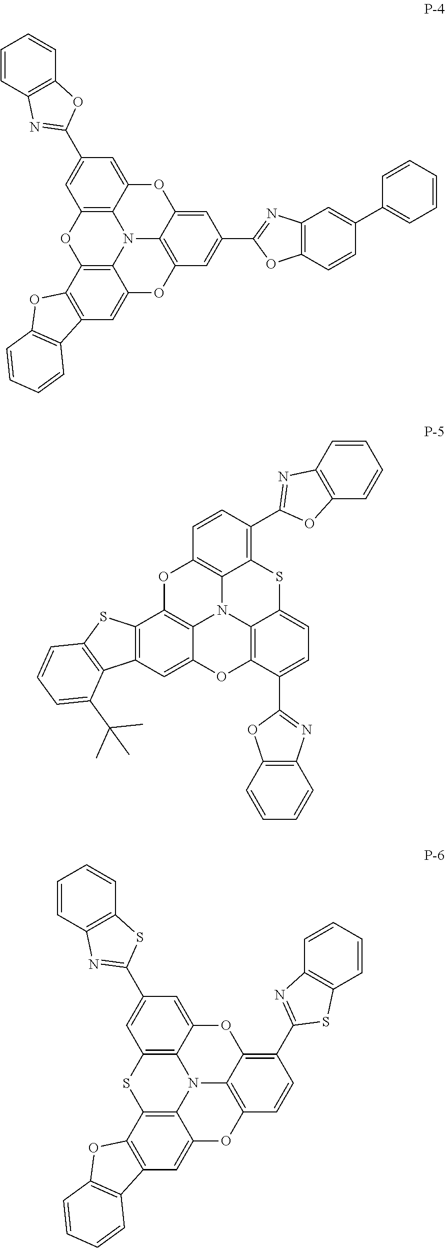 [SMILES: c1ccc(-c2ccc3oc(-c4cc5c6c(c4)Oc4cc7c(oc8ccccc87)c7c4N6c4c(cc(-c6nc8ccccc8o6)cc4O7)O5)nc3c2)cc1 ; CC(C)(C)c1cccc2sc3c4c5c(cc3c12)Oc1c(-c2nc3ccccc3o2)ccc2c1N5c1c(ccc(-c3nc5ccccc5o3)c1S2)O4 ; c1ccc2sc(-c3cc4c5c(c3)Sc3c6c(cc7c3oc3ccccc37)Oc3ccc(-c7nc8ccccc8s7)c(c3N56)O4)nc2c1]

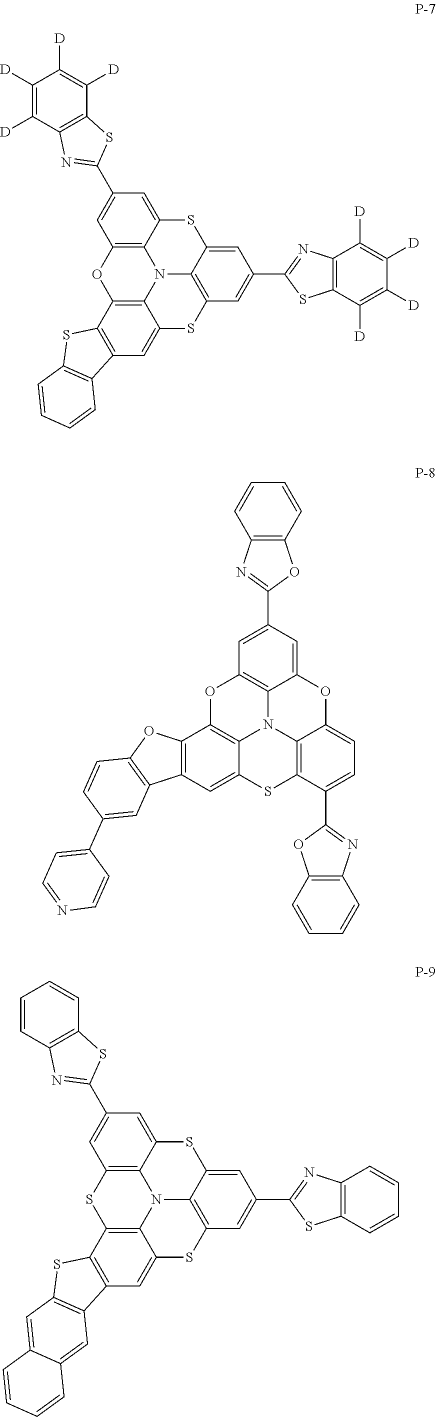 [SMILES: [2H]c1c([2H])c([2H])c2sc(-c3cc4c5c(c3)Sc3cc(-c6nc7c([2H])c([2H])c([2H])c([2H])c7s6)cc6c3N5c3c(cc5c(sc7ccccc75)c3O4)S6)nc2c1[2H] ; c1ccc2oc(-c3cc4c5c(c3)Oc3c6c(cc7c3oc3ccc(-c8ccncc8)cc37)Sc3c(-c7nc8ccccc8o7)ccc(c3N56)O4)nc2c1 ; c1ccc2cc3c(cc2c1)sc1c2c4c(cc13)Sc1cc(-c3nc5ccccc5s3)cc3c1N4c1c(cc(-c4nc5ccccc5s4)cc1S2)S3]

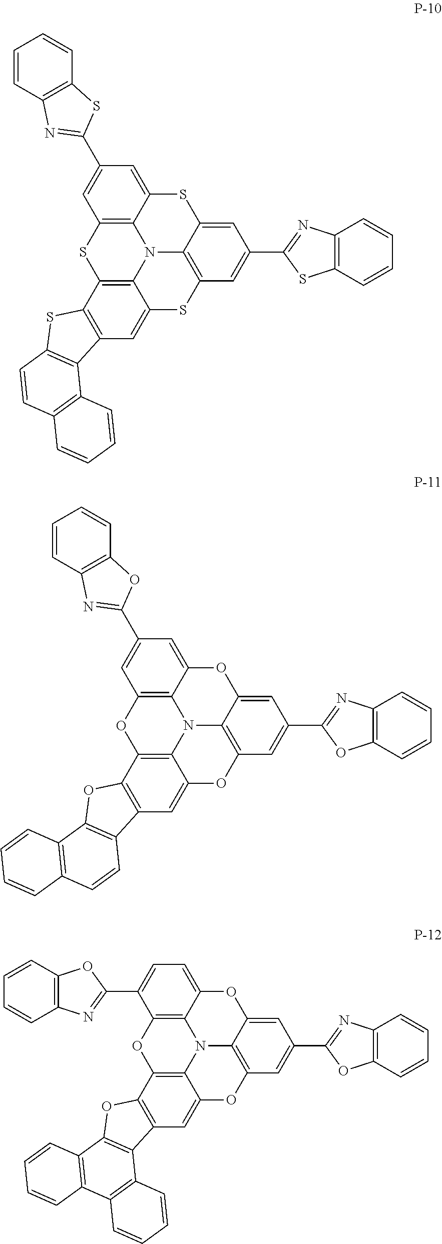

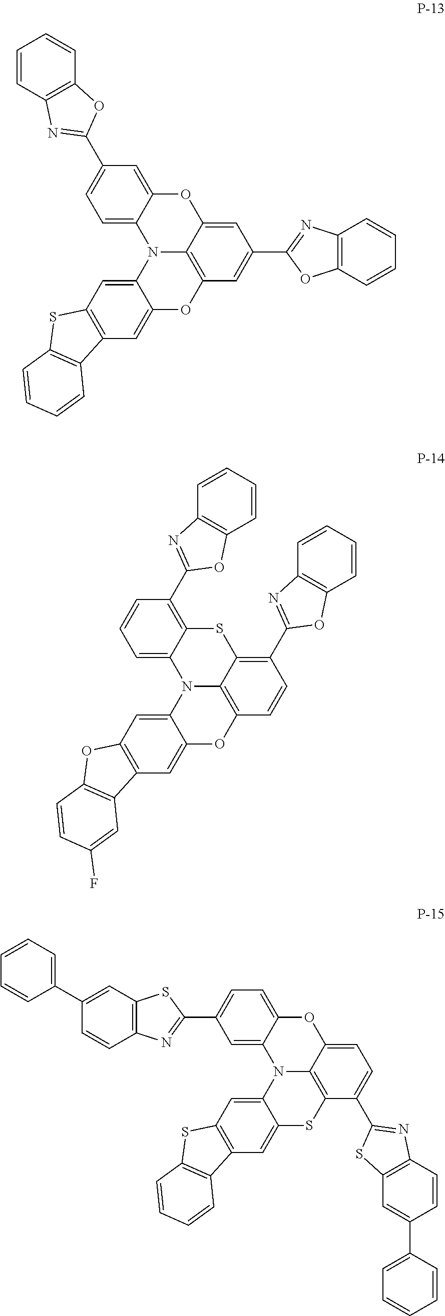 [SMILES: c1ccc2oc(-c3ccc4c(c3)Oc3cc(-c5nc6ccccc6o5)cc5c3N4c3cc4sc6ccccc6c4cc3O5)nc2c1 ; Fc1ccc2oc3cc4c(cc3c2c1)Oc1ccc(-c2nc3ccccc3o2)c2c1N4c1cccc(-c3nc4ccccc4o3)c1S2 ; c1ccc(-c2ccc3nc(-c4ccc5c(c4)N4c6cc7sc8ccccc8c7cc6Sc6c(-c7nc8ccc(-c9ccccc9)cc8s7)ccc(c64)O5)sc3c2)cc1]

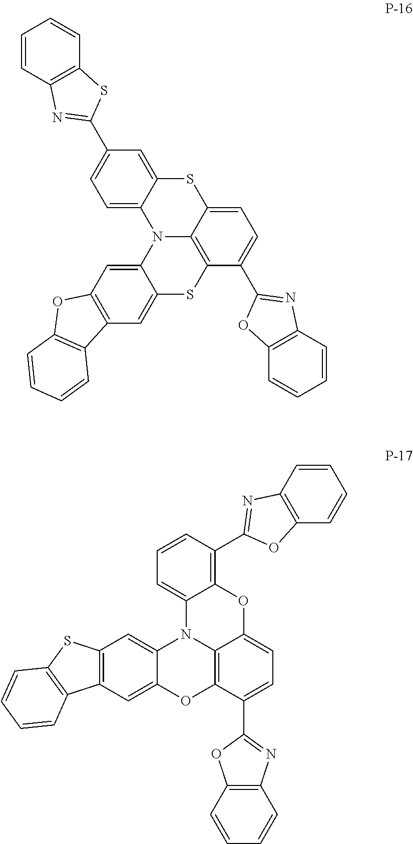 [SMILES: c1ccc2oc(-c3ccc4c5c3Sc3cc6c(cc3N5c3ccc(-c5nc7ccccc7s5)cc3S4)oc3ccccc36)nc2c1 ; c1cc(-c2nc3ccccc3o2)c2c(c1)N1c3cc4sc5ccccc5c4cc3Oc3c(-c4nc5ccccc5o4)ccc(c31)O2]

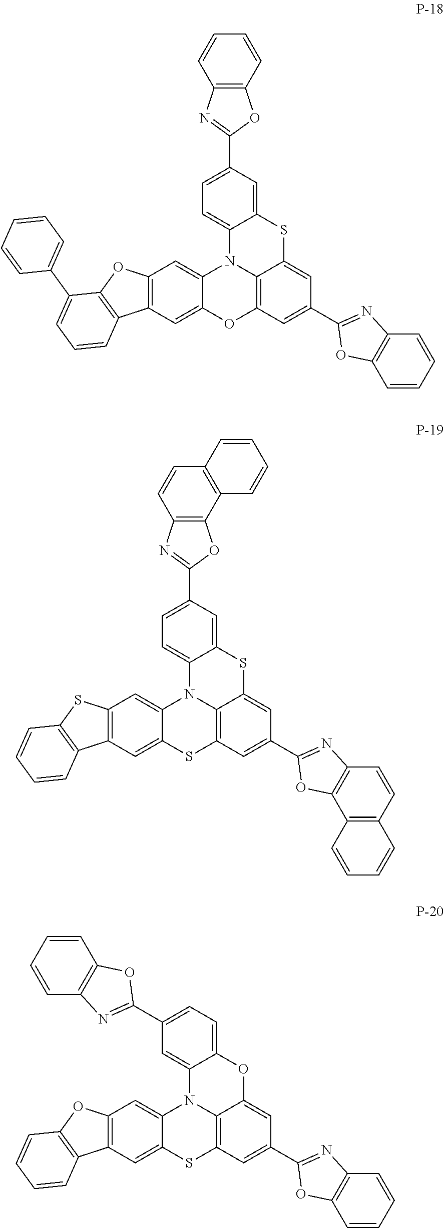 [SMILES: c1ccc(-c2cccc3c2oc2cc4c(cc23)Oc2cc(-c3nc5ccccc5o3)cc3c2N4c2ccc(-c4nc5ccccc5o4)cc2S3)cc1 ; c1ccc2c(c1)ccc1nc(-c3ccc4c(c3)Sc3cc(-c5nc6ccc7ccccc7c6o5)cc5c3N4c3cc4sc6ccccc6c4cc3S5)oc12 ; c1ccc2oc(-c3cc4c5c(c3)Sc3cc6c(cc3N5c3cc(-c5nc7ccccc7o5)ccc3O4)oc3ccccc36)nc2c1]

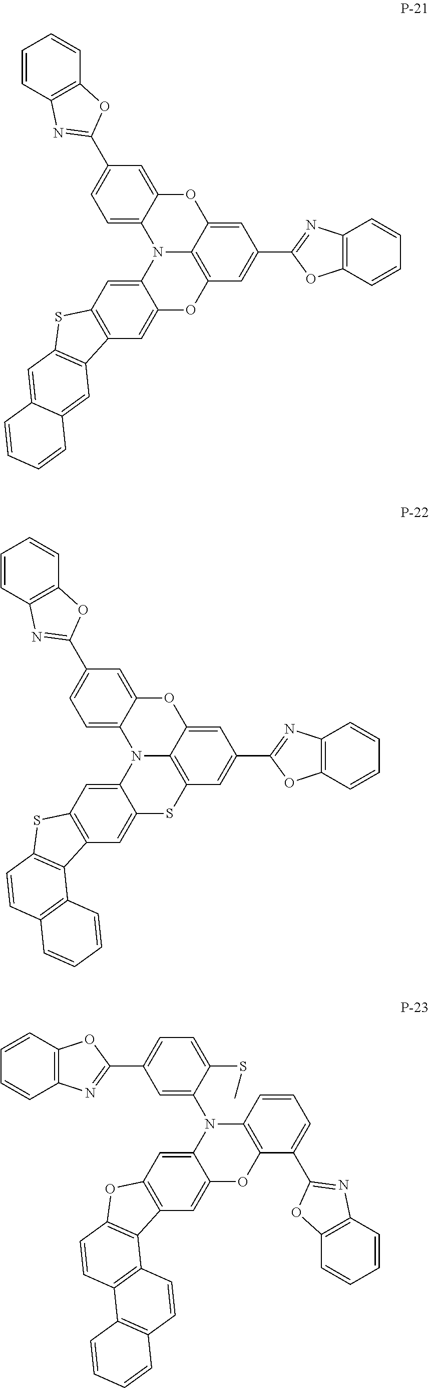 [SMILES: c1ccc2cc3c(cc2c1)sc1cc2c(cc13)Oc1cc(-c3nc4ccccc4o3)cc3c1N2c1ccc(-c2nc4ccccc4o2)cc1O3 ; c1ccc2c(c1)ccc1sc3cc4c(cc3c12)Sc1cc(-c2nc3ccccc3o2)cc2c1N4c1ccc(-c3nc4ccccc4o3)cc1O2 ; CSc1ccc(-c2nc3ccccc3o2)cc1N1c2cc3oc4ccc5c6ccccc6ccc5c4c3cc2Oc2c(-c3nc4ccccc4o3)cccc21]

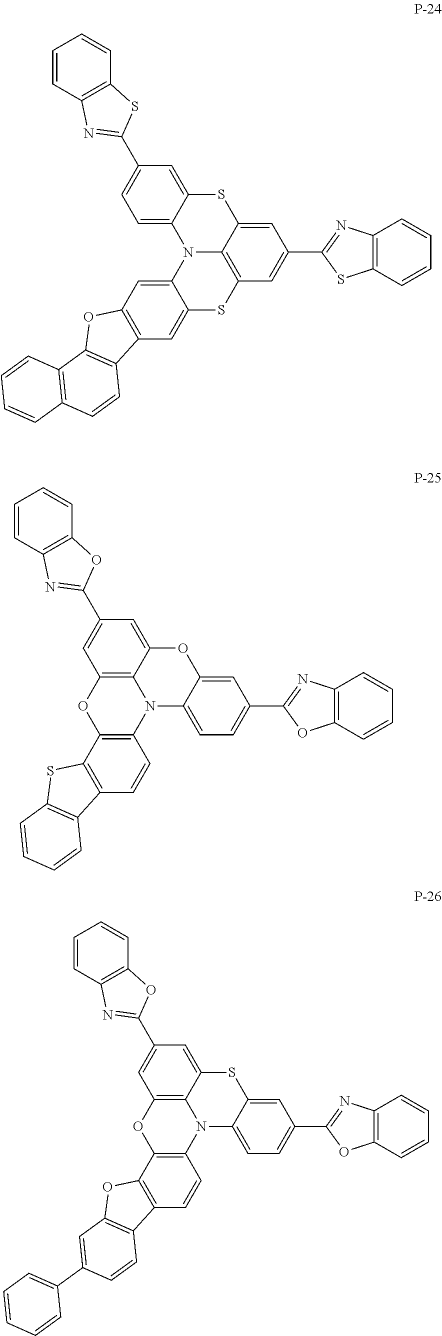 [SMILES: c1ccc2c(c1)ccc1c3cc4c(cc3oc21)N1c2ccc(-c3nc5ccccc5s3)cc2Sc2cc(-c3nc5ccccc5s3)cc(c21)S4 ; c1ccc2oc(-c3ccc4c(c3)Oc3cc(-c5nc6ccccc6o5)cc5c3N4c3ccc4c(sc6ccccc64)c3O5)nc2c1 ; c1ccc(-c2ccc3c(c2)oc2c4c(ccc23)N2c3ccc(-c5nc6ccccc6o5)cc3Sc3cc(-c5nc6ccccc6o5)cc(c32)O4)cc1]

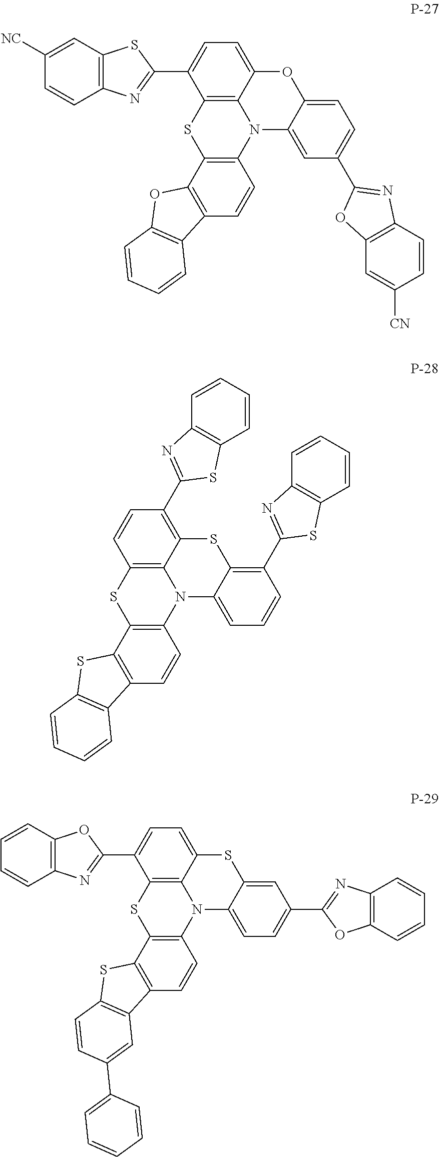 [SMILES: N#Cc1ccc2nc(-c3ccc4c(c3)N3c5ccc6c(oc7ccccc76)c5Sc5c(-c6nc7ccc(C#N)cc7s6)ccc(c53)O4)oc2c1 ; c1cc(-c2nc3ccccc3s2)c2c(c1)N1c3ccc4c(sc5ccccc54)c3Sc3ccc(-c4nc5ccccc5s4)c(c31)S2 ; c1ccc(-c2ccc3sc4c5c(ccc4c3c2)N2c3ccc(-c4nc6ccccc6o4)cc3Sc3ccc(-c4nc6ccccc6o4)c(c32)S5)cc1]

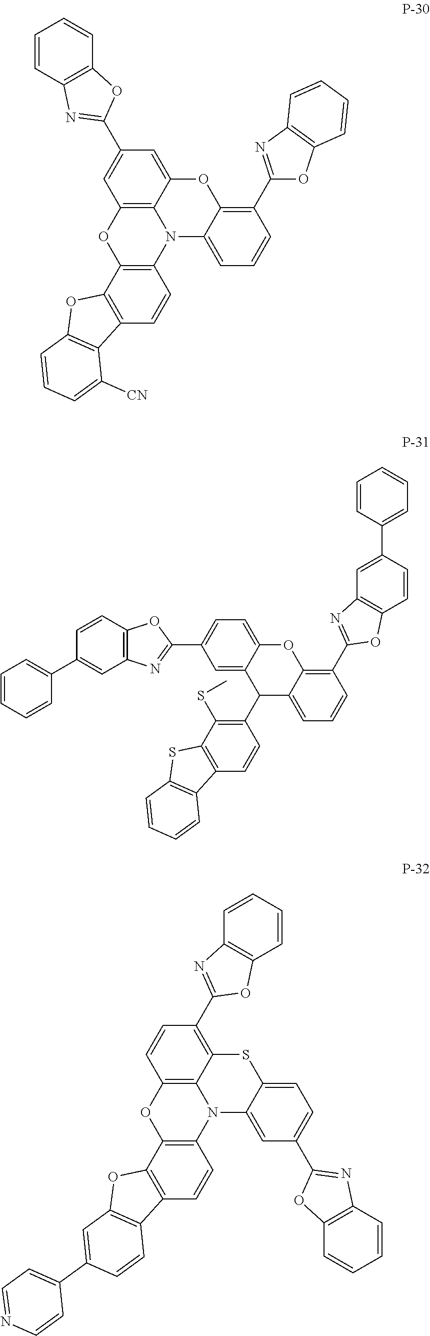 [SMILES: N#Cc1cccc2oc3c4c(ccc3c12)N1c2cccc(-c3nc5ccccc5o3)c2Oc2cc(-c3nc5ccccc5o3)cc(c21)O4 ; CSc1c(C2c3cc(-c4nc5cc(-c6ccccc6)ccc5o4)ccc3Oc3c(-c4nc5cc(-c6ccccc6)ccc5o4)cccc32)ccc2c1sc1ccccc12 ; c1ccc2oc(-c3ccc4c(c3)N3c5ccc6c(oc7cc(-c8ccncc8)ccc76)c5Oc5ccc(-c6nc7ccccc7o6)c(c53)S4)nc2c1]

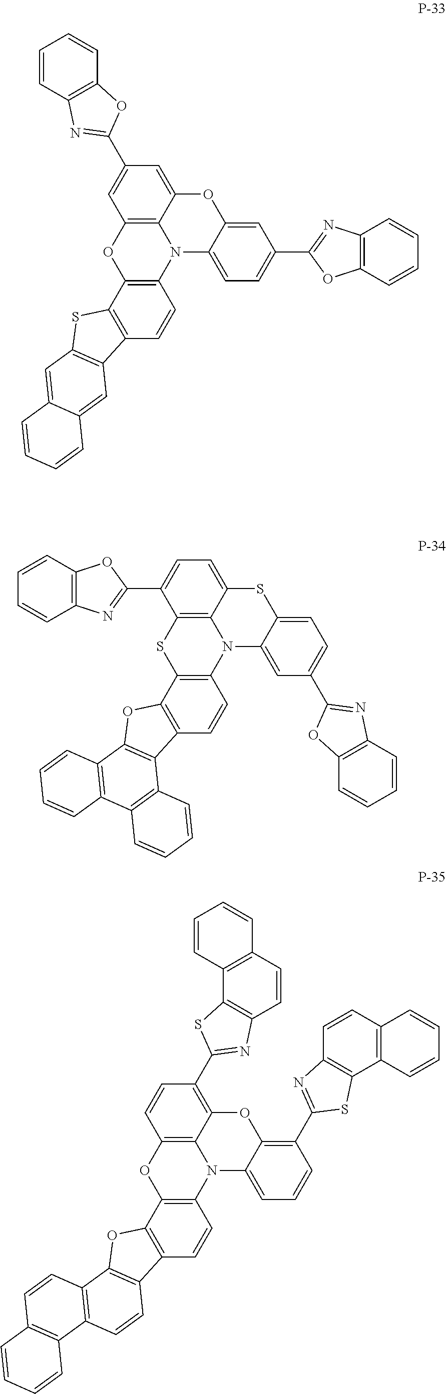 [SMILES: c1ccc2cc3c(cc2c1)sc1c2c(ccc13)N1c3ccc(-c4nc5ccccc5o4)cc3Oc3cc(-c4nc5ccccc5o4)cc(c31)O2 ; c1ccc2oc(-c3ccc4c(c3)N3c5ccc6c(oc7c8ccccc8c8ccccc8c67)c5Sc5c(-c6nc7ccccc7o6)ccc(c53)S4)nc2c1 ; c1cc(-c2nc3ccc4ccccc4c3s2)c2c(c1)N1c3ccc4c(oc5c4ccc4c6ccccc6ccc45)c3Oc3ccc(-c4nc5ccc6ccccc6c5s4)c(c31)O2]

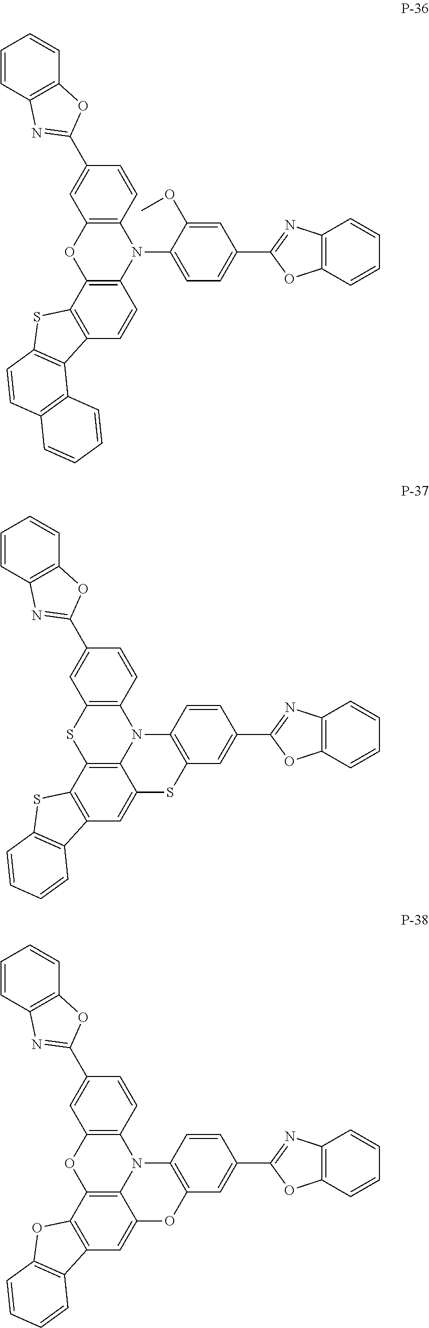 [SMILES: COc1cc(-c2nc3ccccc3o2)ccc1N1c2ccc(-c3nc4ccccc4o3)cc2Oc2c1ccc1c2sc2ccc3ccccc3c21 ; c1ccc2oc(-c3ccc4c(c3)Sc3cc5c(sc6ccccc65)c5c3N4c3ccc(-c4nc6ccccc6o4)cc3S5)nc2c1 ; c1ccc2oc(-c3ccc4c(c3)Oc3cc5c(oc6ccccc65)c5c3N4c3ccc(-c4nc6ccccc6o4)cc3O5)nc2c1]

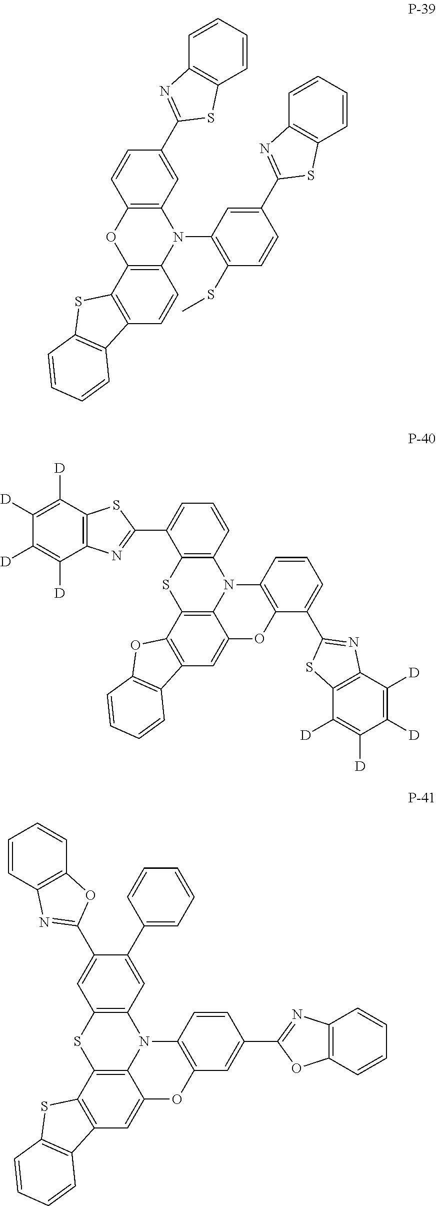 [SMILES: CSc1ccc(-c2nc3ccccc3s2)cc1N1c2cc(-c3nc4ccccc4s3)ccc2Oc2c1ccc1c2sc2ccccc21 ; [2H]c1c([2H])c([2H])c2sc(-c3cccc4c3Oc3cc5c(oc6ccccc65)c5c3N4c3cccc(-c4nc6c([2H])c([2H])c([2H])c([2H])c6s4)c3S5)nc2c1[2H] ; c1ccc(-c2cc3c(cc2-c2nc4ccccc4o2)Sc2c4c(cc5c2sc2ccccc25)Oc2cc(-c5nc6ccccc6o5)ccc2N34)cc1]

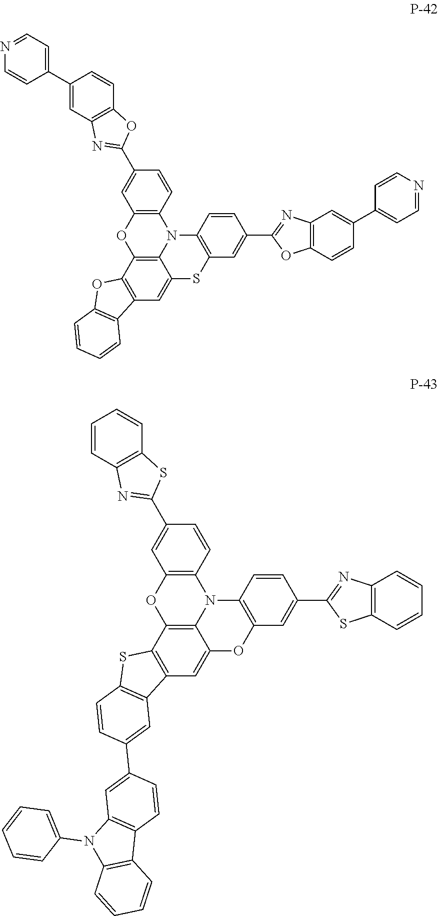 [SMILES: c1ccc2c(c1)oc1c3c4c(cc12)Sc1cc(-c2nc5cc(-c6ccncc6)ccc5o2)ccc1N4c1ccc(-c2nc4cc(-c5ccncc5)ccc4o2)cc1O3 ; c1ccc(-n2c3ccccc3c3ccc(-c4ccc5sc6c7c8c(cc6c5c4)Oc4cc(-c5nc6ccccc6s5)ccc4N8c4ccc(-c5nc6ccccc6s5)cc4O7)cc32)cc1]

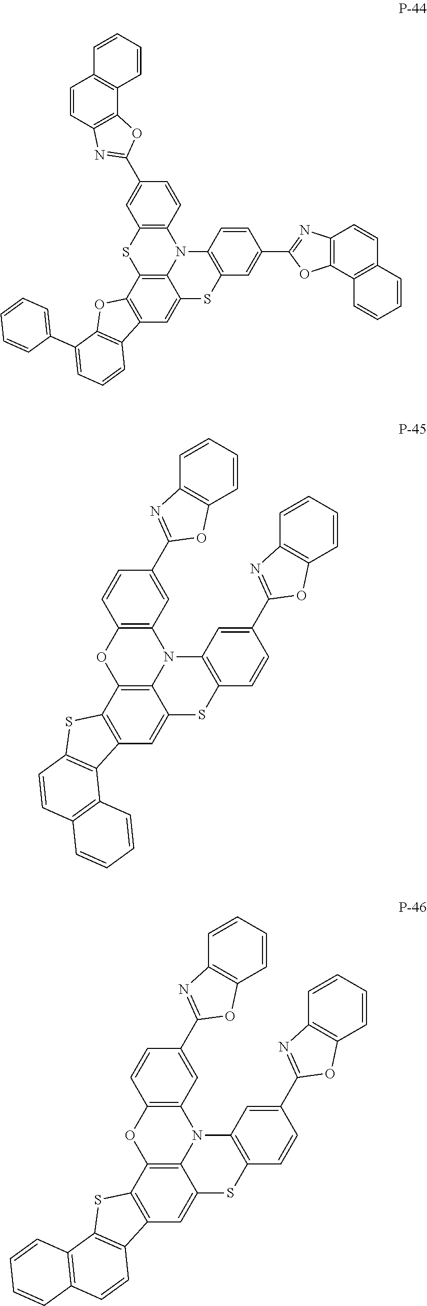 [SMILES: c1ccc(-c2cccc3c2oc2c4c5c(cc23)Sc2cc(-c3nc6ccc7ccccc7c6o3)ccc2N5c2ccc(-c3nc5ccc6ccccc6c5o3)cc2S4)cc1 ; c1ccc2c(c1)ccc1sc3c4c5c(cc3c12)Sc1ccc(-c2nc3ccccc3o2)cc1N5c1cc(-c2nc3ccccc3o2)ccc1O4 ; c1ccc2c(c1)ccc1c3cc4c5c(c3sc21)Oc1ccc(-c2nc3ccccc3o2)cc1N5c1cc(-c2nc3ccccc3o2)ccc1S4]

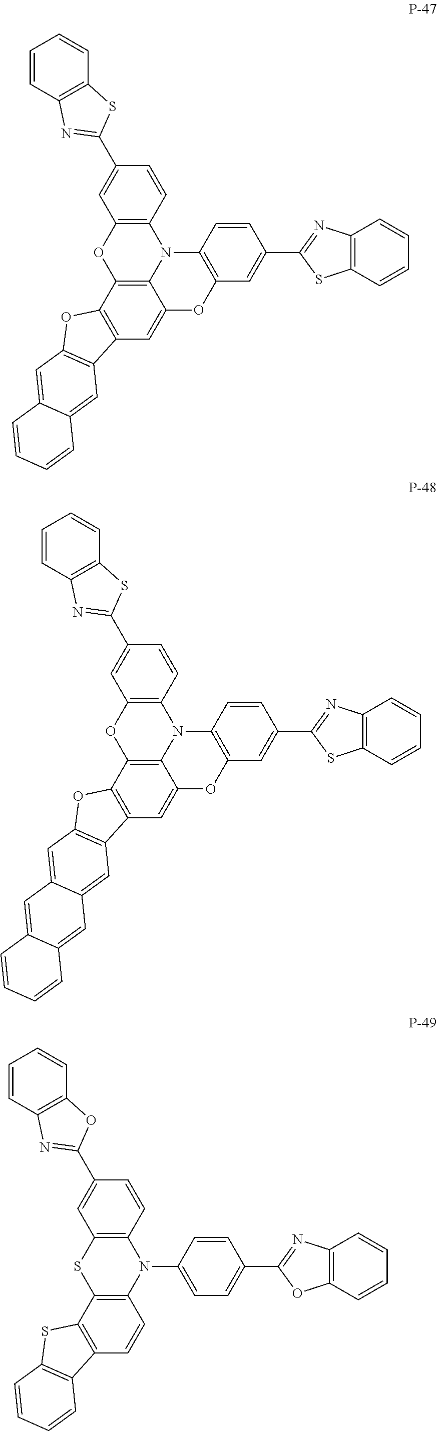 [SMILES: c1ccc2cc3c(cc2c1)oc1c2c4c(cc13)Oc1cc(-c3nc5ccccc5s3)ccc1N4c1ccc(-c3nc4ccccc4s3)cc1O2 ; c1ccc2cc3cc4c(cc3cc2c1)oc1c2c3c(cc14)Oc1cc(-c4nc5ccccc5s4)ccc1N3c1ccc(-c3nc4ccccc4s3)cc1O2 ; c1ccc2oc(-c3ccc(N4c5ccc(-c6nc7ccccc7o6)cc5Sc5c4ccc4c5sc5ccccc54)cc3)nc2c1]

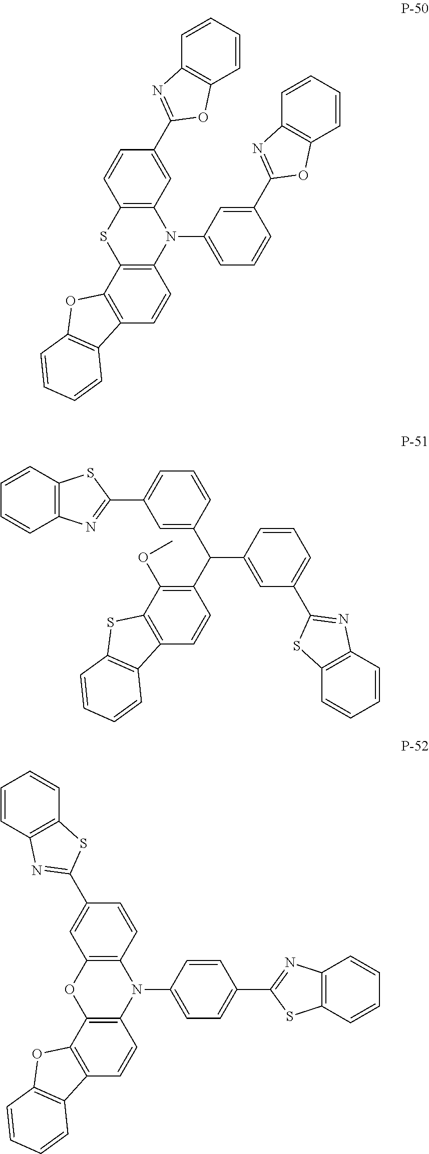 [SMILES: c1cc(-c2nc3ccccc3o2)cc(N2c3cc(-c4nc5ccccc5o4)ccc3Sc3c2ccc2c3oc3ccccc32)c1 ; COc1c(C(c2cccc(-c3nc4ccccc4s3)c2)c2cccc(-c3nc4ccccc4s3)c2)ccc2c1sc1ccccc12 ; c1ccc2sc(-c3ccc(N4c5ccc(-c6nc7ccccc7s6)cc5Oc5c4ccc4c5oc5ccccc54)cc3)nc2c1]

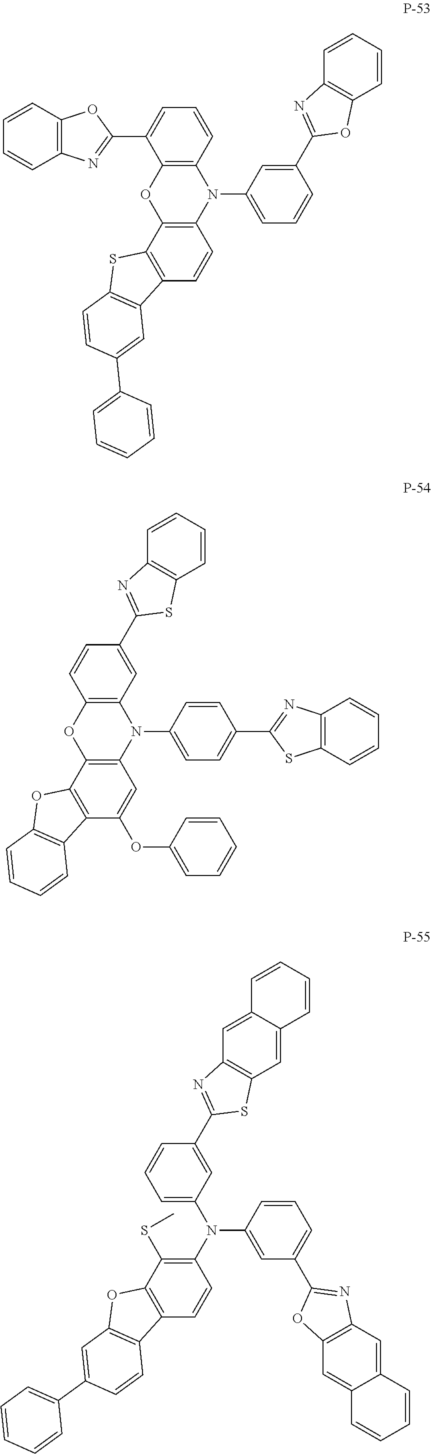 [SMILES: c1ccc(-c2ccc3sc4c5c(ccc4c3c2)N(c2cccc(-c3nc4ccccc4o3)c2)c2cccc(-c3nc4ccccc4o3)c2O5)cc1 ; c1ccc(Oc2cc3c(c4oc5ccccc5c24)Oc2ccc(-c4nc5ccccc5s4)cc2N3c2ccc(-c3nc4ccccc4s3)cc2)cc1 ; CSc1c(N(c2cccc(-c3nc4cc5ccccc5cc4o3)c2)c2cccc(-c3nc4cc5ccccc5cc4s3)c2)ccc2c1oc1cc(-c3ccccc3)ccc12]

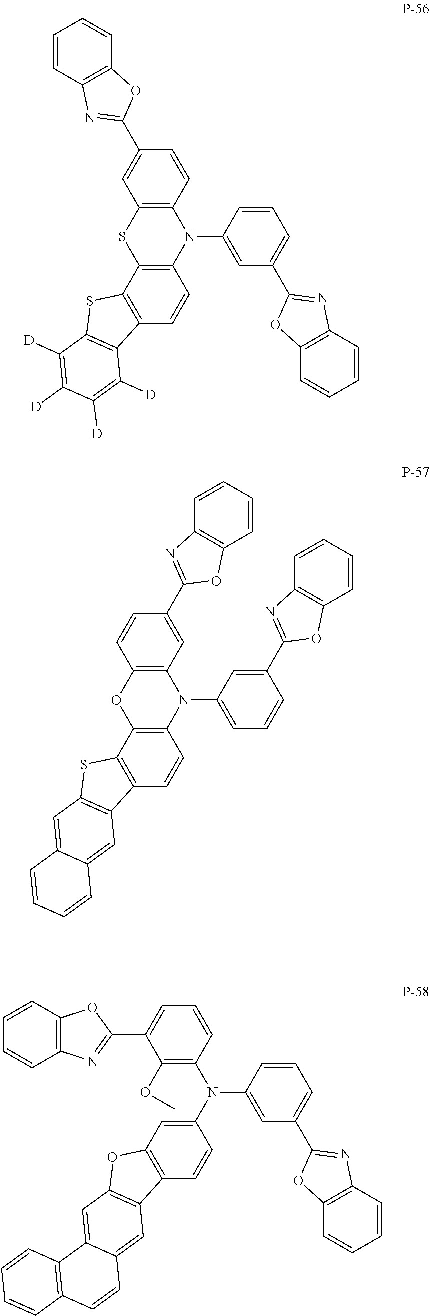 [SMILES: [2H]c1c([2H])c([2H])c2c(sc3c4c(ccc32)N(c2cccc(-c3nc5ccccc5o3)c2)c2ccc(-c3nc5ccccc5o3)cc2S4)c1[2H] ; c1cc(-c2nc3ccccc3o2)cc(N2c3cc(-c4nc5ccccc5o4)ccc3Oc3c2ccc2c3sc3cc4ccccc4cc32)c1 ; COc1c(-c2nc3ccccc3o2)cccc1N(c1cccc(-c2nc3ccccc3o2)c1)c1ccc2c(c1)oc1cc3c(ccc4ccccc43)cc12]

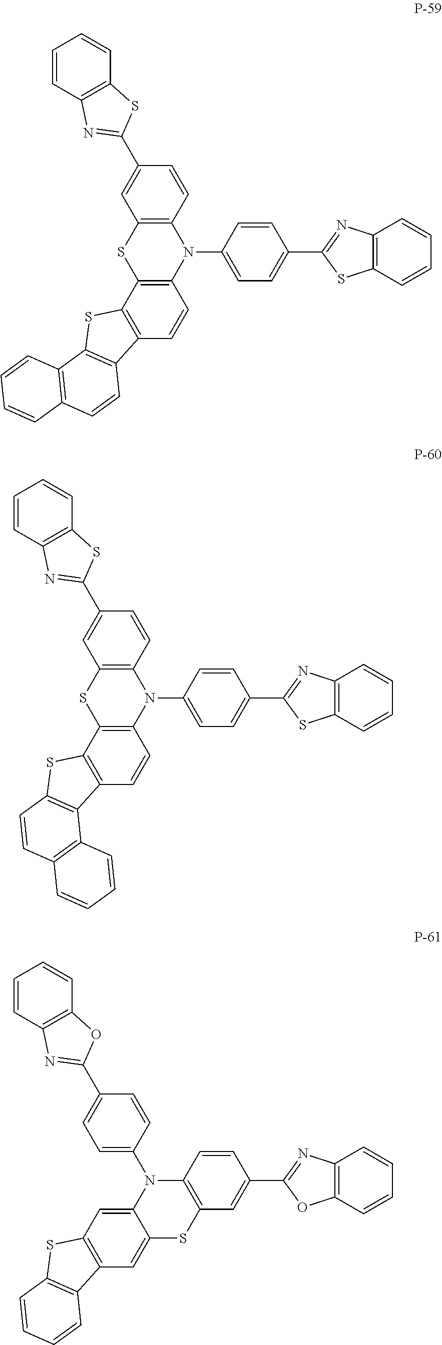 [SMILES: c1ccc2c(c1)ccc1c3ccc4c(c3sc21)Sc1cc(-c2nc3ccccc3s2)ccc1N4c1ccc(-c2nc3ccccc3s2)cc1 ; c1ccc2c(c1)ccc1sc3c4c(ccc3c12)N(c1ccc(-c2nc3ccccc3s2)cc1)c1ccc(-c2nc3ccccc3s2)cc1S4 ; c1ccc2oc(-c3ccc(N4c5ccc(-c6nc7ccccc7o6)cc5Sc5cc6c(cc54)sc4ccccc46)cc3)nc2c1]

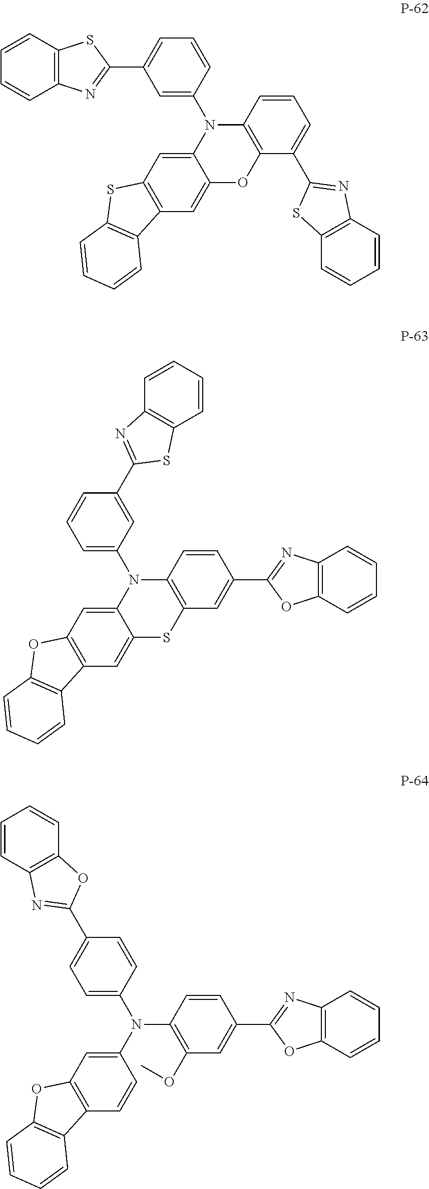 [SMILES: c1cc(-c2nc3ccccc3s2)cc(N2c3cc4sc5ccccc5c4cc3Oc3c(-c4nc5ccccc5s4)cccc32)c1 ; c1cc(-c2nc3ccccc3s2)cc(N2c3ccc(-c4nc5ccccc5o4)cc3Sc3cc4c(cc32)oc2ccccc24)c1 ; COc1cc(-c2nc3ccccc3o2)ccc1N(c1ccc(-c2nc3ccccc3o2)cc1)c1ccc2c(c1)oc1ccccc12]

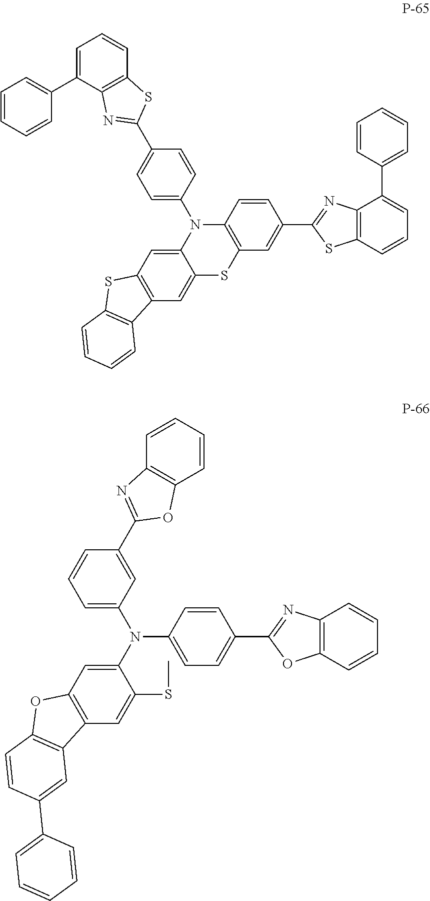 [SMILES: c1ccc(-c2cccc3sc(-c4ccc(N5c6ccc(-c7nc8c(-c9ccccc9)cccc8s7)cc6Sc6cc7c(cc65)sc5ccccc57)cc4)nc23)cc1 ; CSc1cc2c(cc1N(c1ccc(-c3nc4ccccc4o3)cc1)c1cccc(-c3nc4ccccc4o3)c1)oc1ccc(-c3ccccc3)cc12]

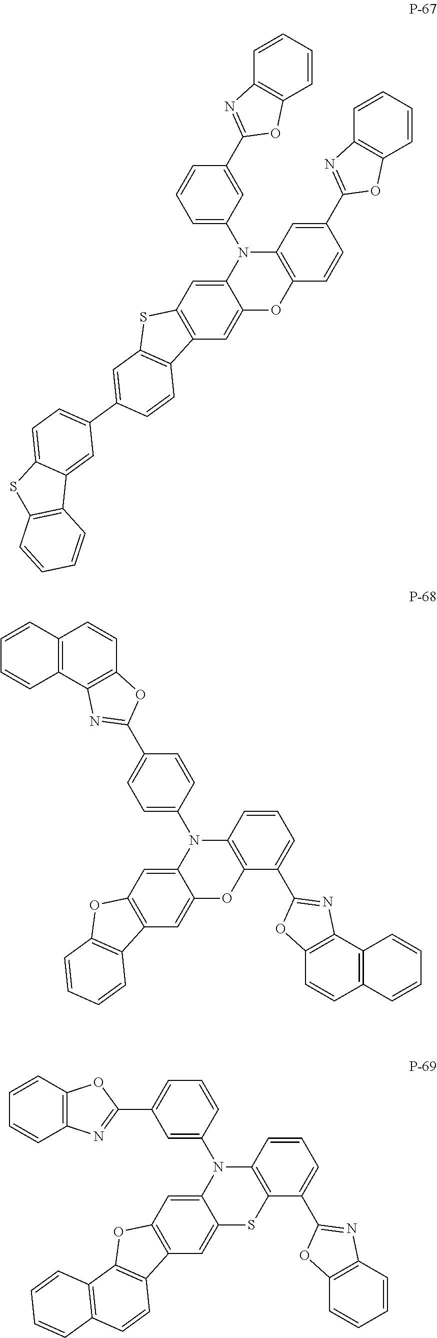 [SMILES: c1cc(-c2nc3ccccc3o2)cc(N2c3cc(-c4nc5ccccc5o4)ccc3Oc3cc4c(cc32)sc2cc(-c3ccc5sc6ccccc6c5c3)ccc24)c1 ; c1cc(-c2nc3c(ccc4ccccc43)o2)c2c(c1)N(c1ccc(-c3nc4c(ccc5ccccc54)o3)cc1)c1cc3oc4ccccc4c3cc1O2 ; c1cc(-c2nc3ccccc3o2)cc(N2c3cc4oc5c6ccccc6ccc5c4cc3Sc3c(-c4nc5ccccc5o4)cccc32)c1]

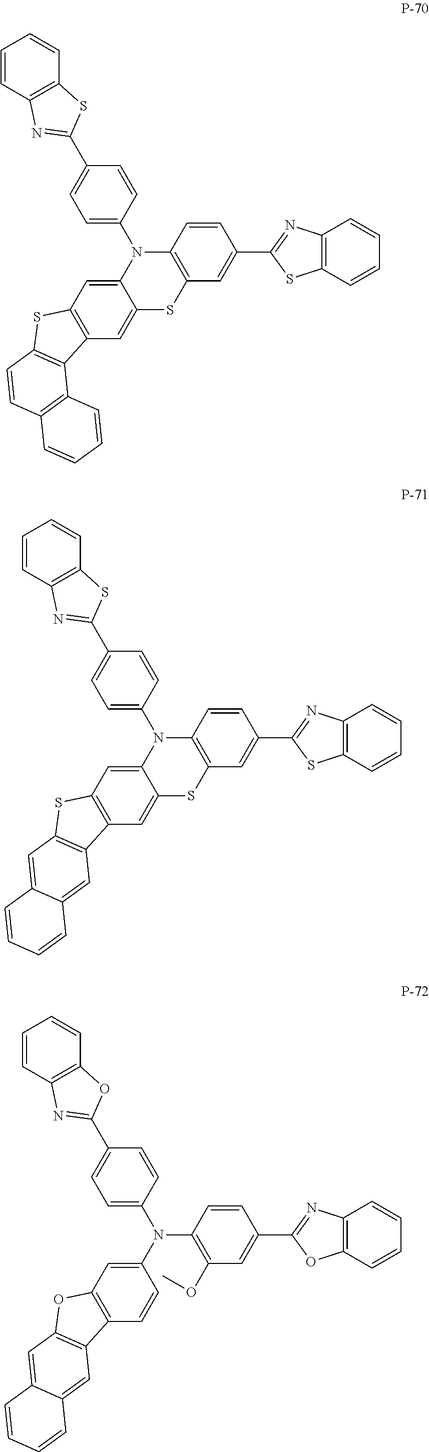 [SMILES: c1ccc2c(c1)ccc1sc3cc4c(cc3c12)Sc1cc(-c2nc3ccccc3s2)ccc1N4c1ccc(-c2nc3ccccc3s2)cc1 ; c1ccc2cc3c(cc2c1)sc1cc2c(cc13)Sc1cc(-c3nc4ccccc4s3)ccc1N2c1ccc(-c2nc3ccccc3s2)cc1 ; COc1cc(-c2nc3ccccc3o2)ccc1N(c1ccc(-c2nc3ccccc3o2)cc1)c1ccc2c(c1)oc1cc3ccccc3cc12]

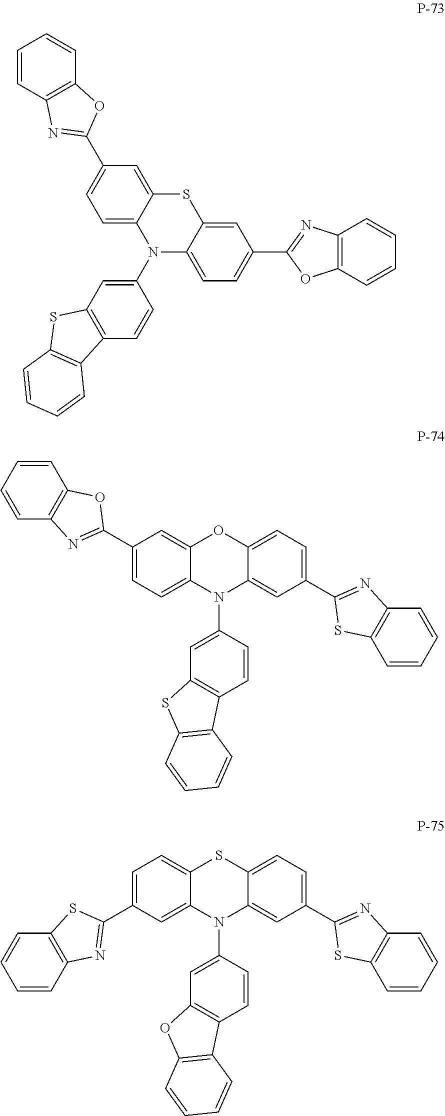

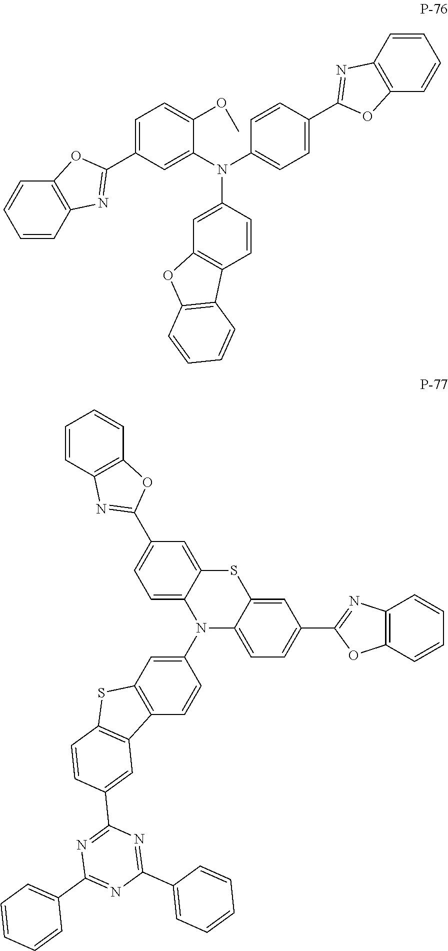 [SMILES: COc1ccc(-c2nc3ccccc3o2)cc1N(c1ccc(-c2nc3ccccc3o2)cc1)c1ccc2c(c1)oc1ccccc12 ; c1ccc(-c2nc(-c3ccccc3)nc(-c3ccc4sc5cc(N6c7ccc(-c8nc9ccccc9o8)cc7Sc7cc(-c8nc9ccccc9o8)ccc76)ccc5c4c3)n2)cc1]

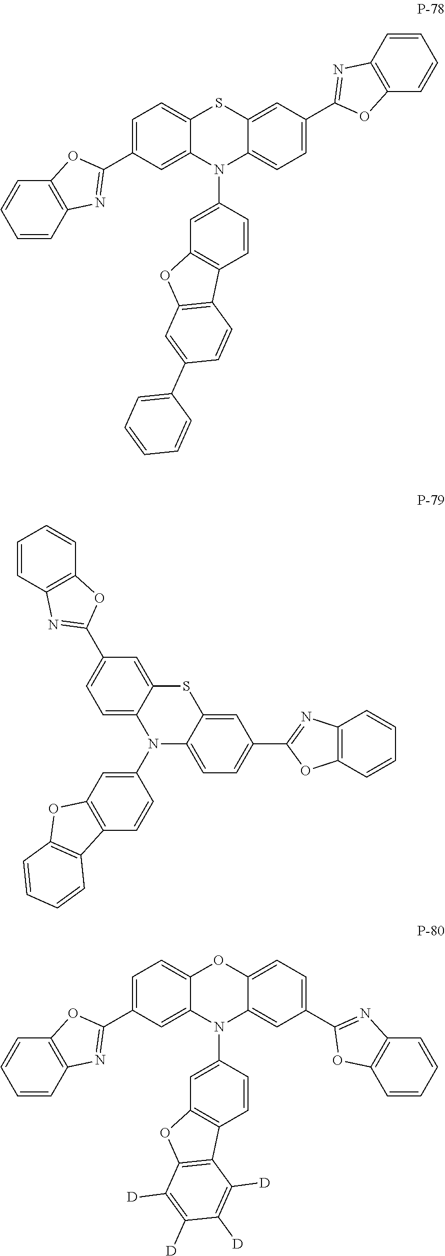 [SMILES: c1ccc(-c2ccc3c(c2)oc2cc(N4c5ccc(-c6nc7ccccc7o6)cc5Sc5ccc(-c6nc7ccccc7o6)cc54)ccc23)cc1 ; c1ccc2oc(-c3ccc4c(c3)Sc3cc(-c5nc6ccccc6o5)ccc3N4c3ccc4c(c3)oc3ccccc34)nc2c1 ; [2H]c1c([2H])c([2H])c2c(oc3cc(N4c5cc(-c6nc7ccccc7o6)ccc5Oc5ccc(-c6nc7ccccc7o6)cc54)ccc32)c1[2H]]

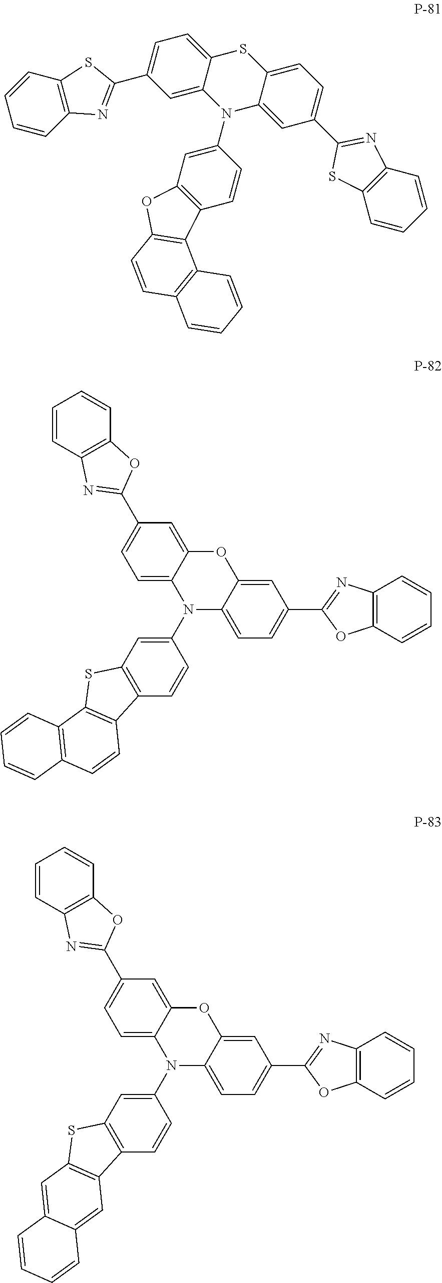 [SMILES: c1ccc2c(c1)ccc1oc3cc(N4c5cc(-c6nc7ccccc7s6)ccc5Sc5ccc(-c6nc7ccccc7s6)cc54)ccc3c12 ; c1ccc2c(c1)ccc1c3ccc(N4c5ccc(-c6nc7ccccc7o6)cc5Oc5cc(-c6nc7ccccc7o6)ccc54)cc3sc21 ; c1ccc2cc3c(cc2c1)sc1cc(N2c4ccc(-c5nc6ccccc6o5)cc4Oc4cc(-c5nc6ccccc6o5)ccc42)ccc13]

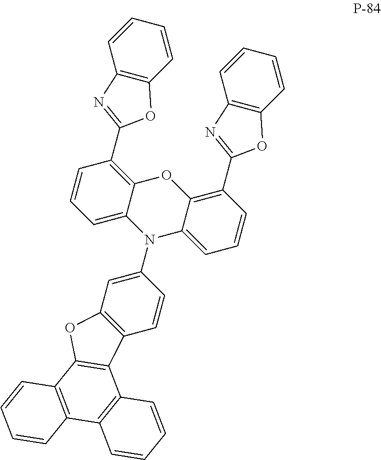 [SMILES: c1cc(-c2nc3ccccc3o2)c2c(c1)N(c1ccc3c(c1)oc1c4ccccc4c4ccccc4c31)c1cccc(-c3nc4ccccc4o3)c1O2]

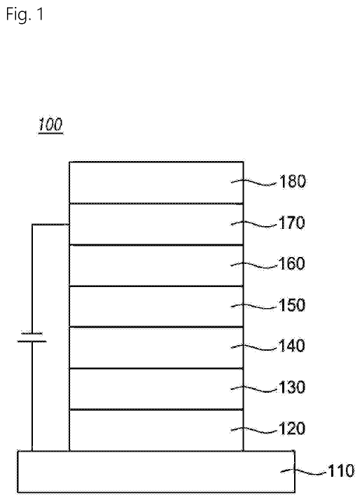

Referring to FIG. 1, the organic electronic element (100) according to the present invention includes a first electrode (110), a second electrode (170),

an organic material layer and a light efficiency enhancing layer formed between the first electrode (110) and the second electrode (170), and the light efficiency enhancing layer (180) is formed on one side of both surfaces of the first electrode (110) or the second electrode (170) that is not in contact with the organic material layer, and the organic material layer or the light efficiency enhancing layer includes a single compound or 2 or more compounds represented by Formula 1.

In this case, the first electrode (110) may be an anode or a positive electrode, and the second electrode (170) may be a cathode or a negative electrode. In the case of an inverted type, the first electrode may be a cathode and the second electrode may be an anode.

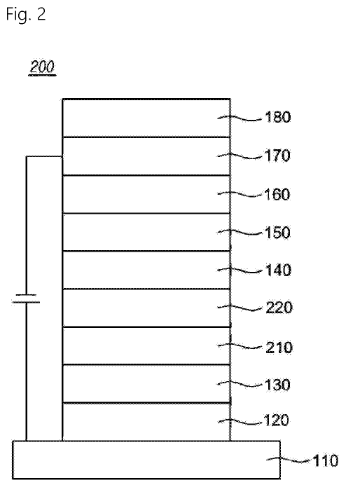

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2)

Also, the organic electronic element according to an embodiment of the present invention may further include a protective layer. The compound according to an embodiment of the present invention may be used as a material for the host or dopant of the organic material layer, that is, the hole injection layer (120), a hole transport layer (130), an emitting-auxiliary layer (220), the electron transport auxiliary layer, the electron transport layer (150), an electron injection layer (160), or an emitting layer (140), or may be used as a material for the light efficiency enhancing layer. Preferably, for example, the compound according to Formula 1 of the present invention may be used as a material for the light efficiency enhancing layer.

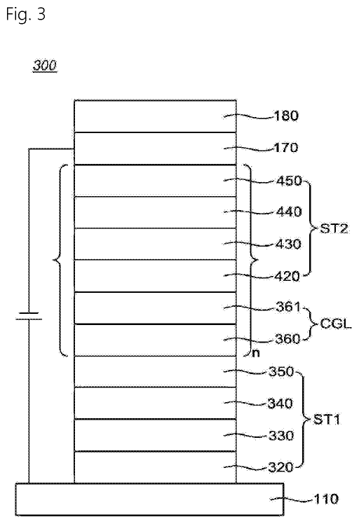

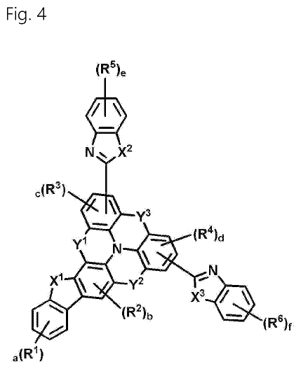

The organic material layer may include 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important.

The organic electroluminescent device according to an embodiment of the present invention may be manufactured using a PVD (physical vapor deposition) method. For example, it may be manufactured by depositing a metal or a metal oxide having conductivity or an alloy thereof on a substrate to form an anode, forming an organic material layer including the hole injection layer (120), the hole transport layer (130), the emitting layer (140), the electron transport layer (150) and the electron injection layer (160) thereon, and then depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process.

As another specific example, the present invention provides a light efficiency enhancing layer composition comprising the compound represented by Formula 1 above.

Also, in the light efficiency enhancing layer, a compound of the same kind or different kinds of the compound represented by Formula 1 is mixed and used.

Also, the present invention provides an electronic device comprising a display device including the organic electronic element; and a control unit for driving the display device;

In another aspect, the organic electronic element is at least one of an organic electroluminescent device, an organic solar cell, an organic photoreceptor, an organic transistor, and a device for monochromatic or white lighting. At this time, the electronic device may be a current or future wired/wireless communication terminal, and covers all kinds of electronic devices including mobile communication terminals such as mobile phones, a personal digital assistant (PDA), an electronic dictionary, a point-to-multipoint (PMP), a remote controller, a navigation unit, a game player, various kinds of TVs, and various kinds of computers.

Hereinafter, a synthesis example of the compound represented by Formula 1 of the present invention and a manufacturing example of an organic electronic element of the present invention will be described in detail with reference to Examples, but the present invention is not limited to the following Examples.

### Synthesis Example 1

The compound represented by Formula 1 according to the present invention (Final product) may be prepared by reacting as shown in Scheme 1 below, but is not limited thereto.

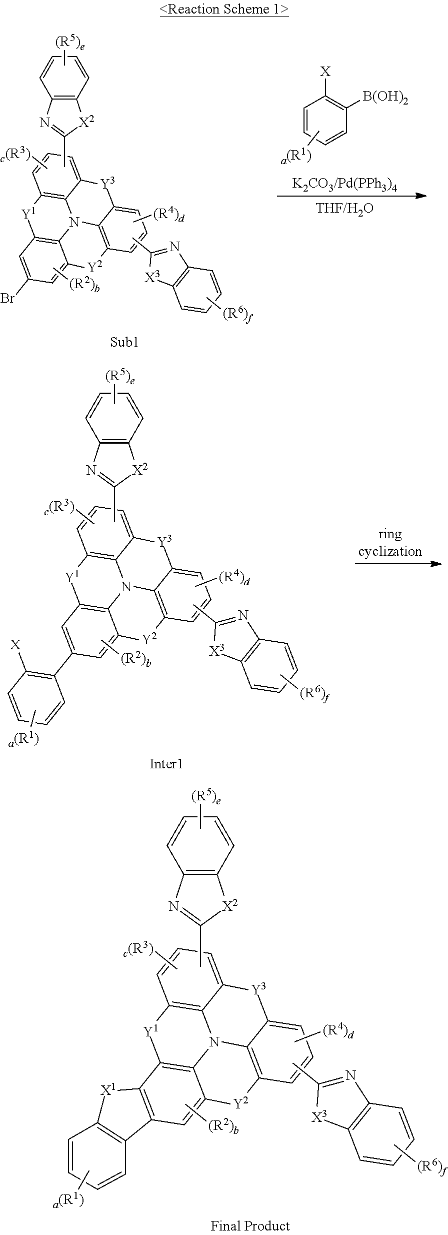

(wherein, X is —OH or —SMe.)

### Synthesis of Sub 1

Sub 1 of Reaction Scheme 1 may be synthesized by the reaction route of Reaction Scheme 2 below, but is not limited thereto.

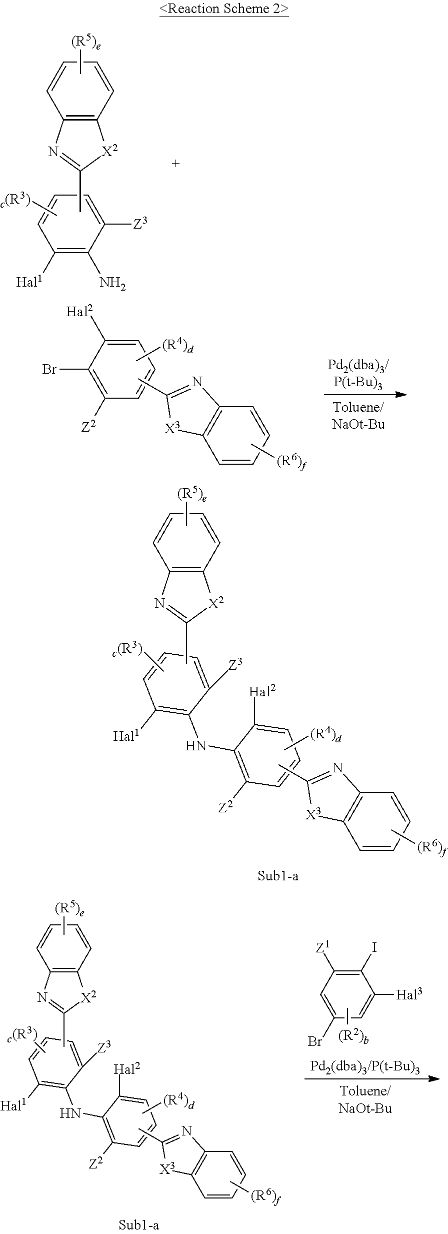

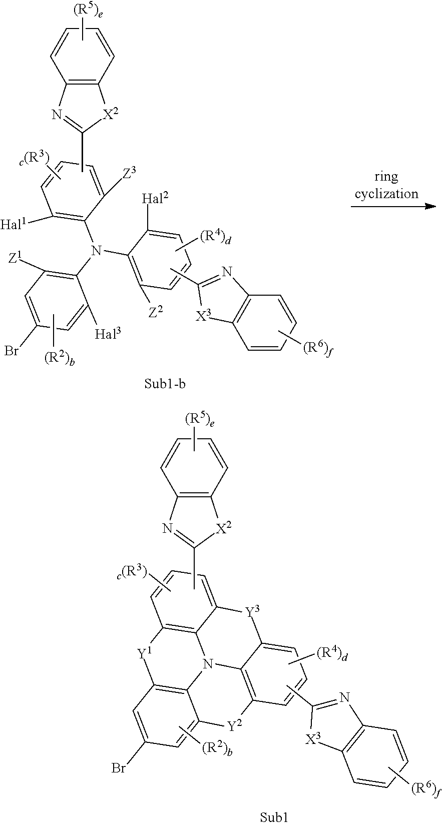

(wherein, Hal¹ to Hal³ are F, Cl or absent, and Z¹ to Z³ are —OH, —SH, or absent.)

### 1. Synthesis Example of Sub1-1

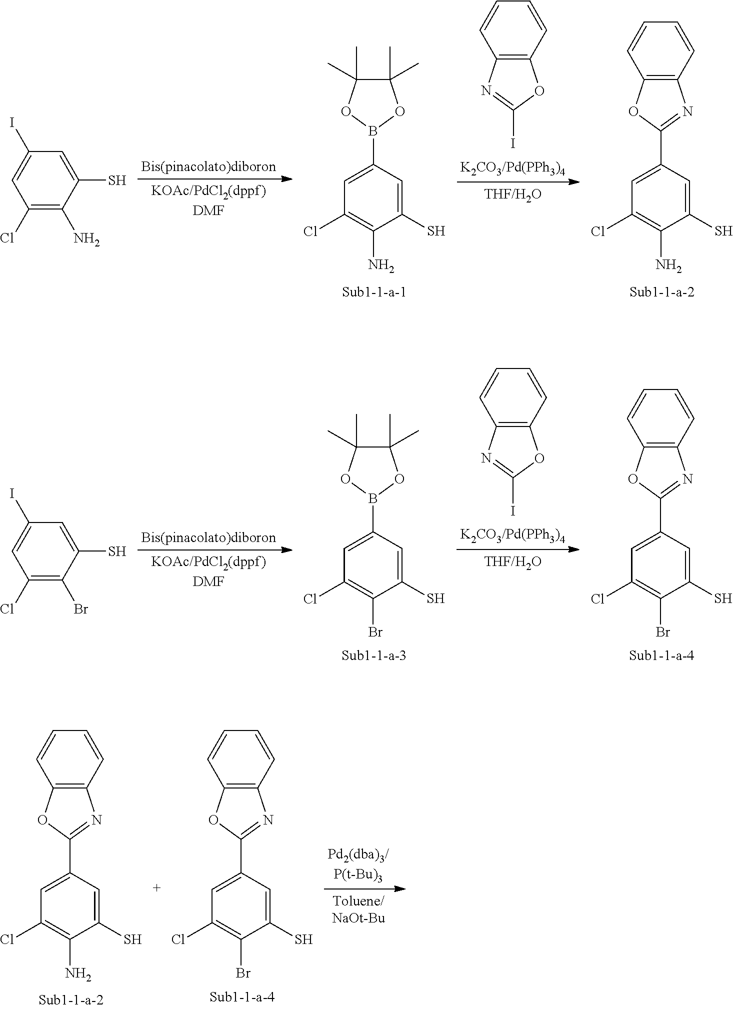

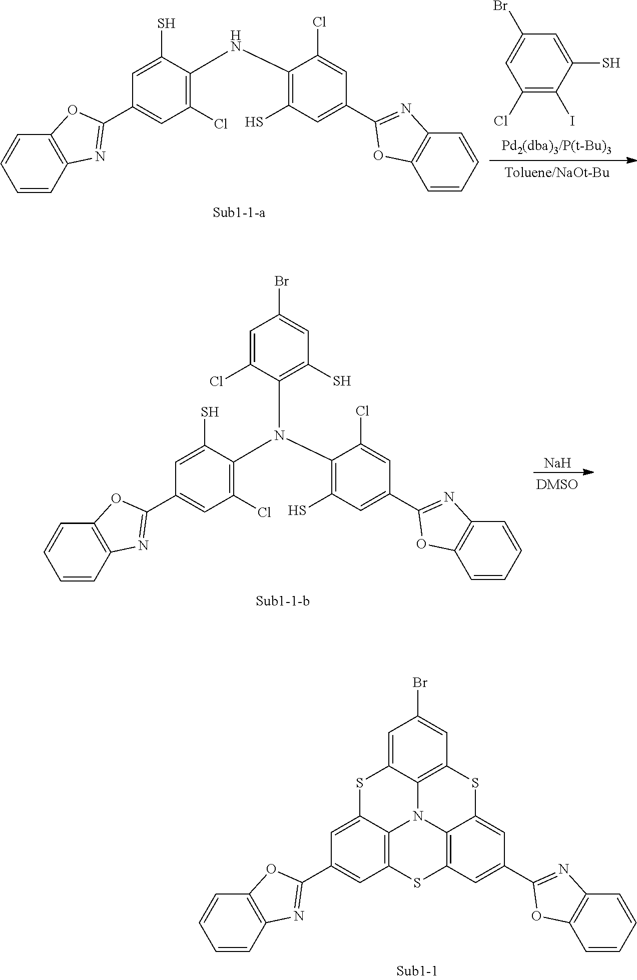

### (1) Synthesis of Sub1-1-a-1

After dissolving 2-amino-3-chloro-5-iodobenzenethiol (50.0 g, 175 mmol) in DMF (Dimethylformamide) (880 mL) in a round-bottom flask, bis(pinacolato)diboron (48.9 g, 193 mmol), KOAc (51.6 g, 525 mmol), PdCl₂(dppf) (3.84 g, 5.25 mmol) were added and stirred at 120° C. When the reaction is completed, DMF is removed through distillation, extracted with CH₂Cl₂ and water, and the organic layer is dried over MgSO₄ and concentrated. Thereafter, the resulting organic material was recrystallized by silicagel column to obtain 35.5 g of a product. (Yield 71%)

### (2) Synthesis of Sub1-1-a-2

After dissolving Sub1-1-a-1 (35.5 g, 124 mmol) obtained in the above synthesis in THF (Tetrahydrofuran) (620 mL) in a round-bottom flask, 2-iodobenzo[d]oxazole (30.5 g, 124 mmol), K₂CO₃ (51.5 g, 373 mmol), Pd(PPh₃)₄ (8.62 g, 7.46 mmol), and water (310 mL) were added and stirred at 80° C. When the reaction is complete, the mixture is extracted with CH₂Cl₂ and water, and the organic layer is dried over MgSO₄ and concentrated. Thereafter, the resulting organic material was recrystallized using silicagel column to obtain 28.9 g of a product. (Yield 84%)

### (3) Synthesis of Sub1-1-a-3

2-bromo-3-chloro-5-iodobenzenethiol (50.0 g, 143 mmol), bis(pinacolato)diboron (40.0 g, 157 mmol), KOAc (42.1 g, 429 mmol), PdCl₂(dppf) (3.14 g, 4.29 mmol) were tested in the same manner as in Sub1-1-a-1 above to obtain 36.5 g of the product. (Yield 73%)

### (4) Synthesis of Sub1-1-a-4

After dissolving Sub1-1-a-3 (36.5 g, 105 mmol) obtained in the above synthesis in THF (520 mL) in a round-bottom flask, and 2-iodobenzo[d]oxazole (25.6 g, 105 mmol), K₂CO₃ (43.3 g, 313 mmol), Pd(PPh₃)₄ (7.24 g, 6.27 mmol), water (260 mL) were added and an experiment was performed in the same manner as in Sub1-1-a-2 to obtain 28.8 g of a product. (Yield 81%)

### (5) Synthesis of Sub1-1-a-a

After dissolving Sub1-1-a-2 (23.4 g, 84.6 mmol) obtained in the above synthesis in toluene (423 mL) in a round-bottom flask, Sub 1-1-a-4 (28.8 g, 84.6 mmol) obtained in the above synthesis, Pd₂(dba)₃ (2.32 g, 2.54 mmol), P(t-Bu)₃ (1.03 g, 5.08 mmol), NaOt-Bu (16.3 g, 169 mmol) were added and the reaction proceeds at 80° C. When the reaction is complete, the mixture is extracted with CH₂Cl₂ and water, and the organic layer is dried over MgSO₄ and concentrated. Thereafter, the resulting organic material was recrystallized using silicagel column to obtain 34.0 g of a product. (yield 75%)

### (6) Synthesis of Sub1-1-b

After dissolving Sub1-1-a (34.0 g, 63.5 mmol) obtained in the above synthesis in toluene (320 mL) in a round-bottom flask, 5-bromo-3-chloro-2-iodobenzenethiol (22.2 g, 63.5 mmol) Pd₂(dba)₃ (1.74 g, 1.90 mmol), P(t-Bu)₃ (0.77 g, 3.81 mmol), NaOt-Bu (12.2 g, 127 mmol) were added and the reaction was carried out at 80° C. When the reaction is complete, the mixture is extracted with CH₂Cl₂ and water, and the organic layer is dried over MgSO₄ and concentrated. Thereafter, the resulting organic material was recrystallized using silicagel column to obtain 36.6 g of a product. (yield 76%)

### (7) Synthesis of Sub1-1

In a round bottom flask, NaH (90%) (7.72 g, 322 mmol) was dissolved in DMSO (Dimethyl sulfoxide) (322 mL), and then cooled to 0° C. After dissolving Sub1-1-b (36.6 g, 48.2 mmol) obtained in the above synthesis in DMSO (161 mL), it was added dropwise to a round flask and stirred for 1 hour. Then, the mixture was stirred at 90° C. for 2 hours. When the reaction is complete, extraction is performed with ethylacetate and water, and the organic layer is dried over MgSO4 and concentrated. Thereafter, the resulting organic material was recrystallized by silicagel column to obtain 14.4 g of a product. (Yield 46%)

### 2. Synthesis Example of Sub1-4

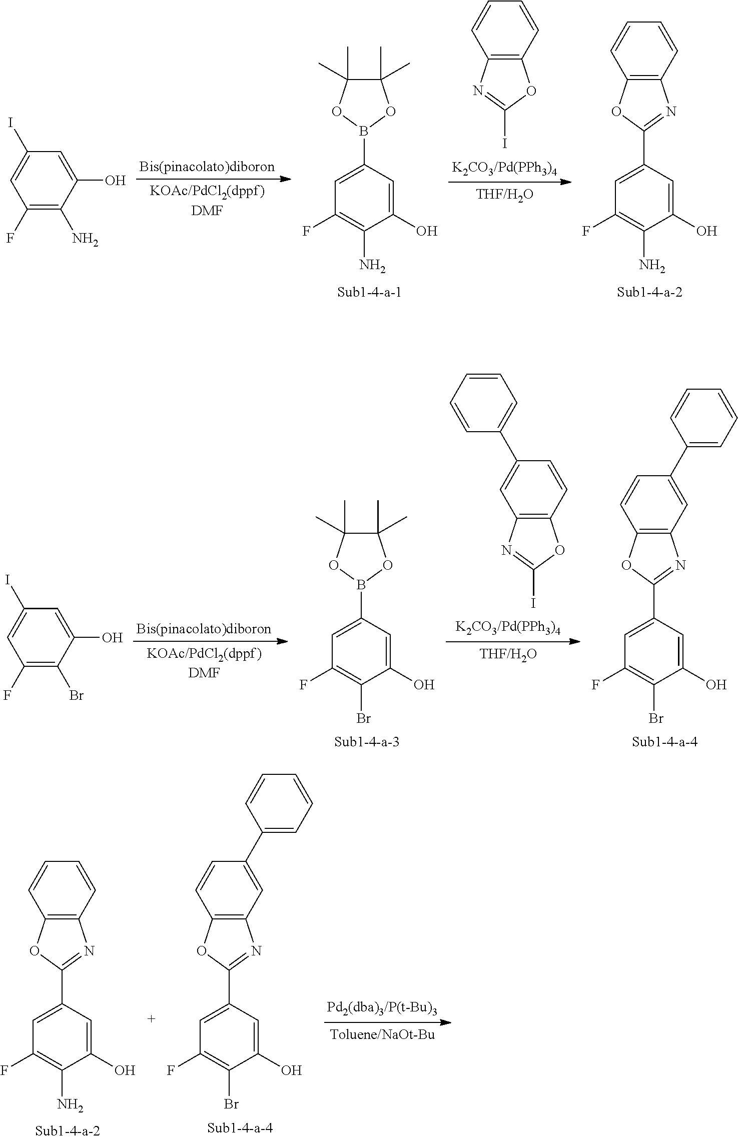

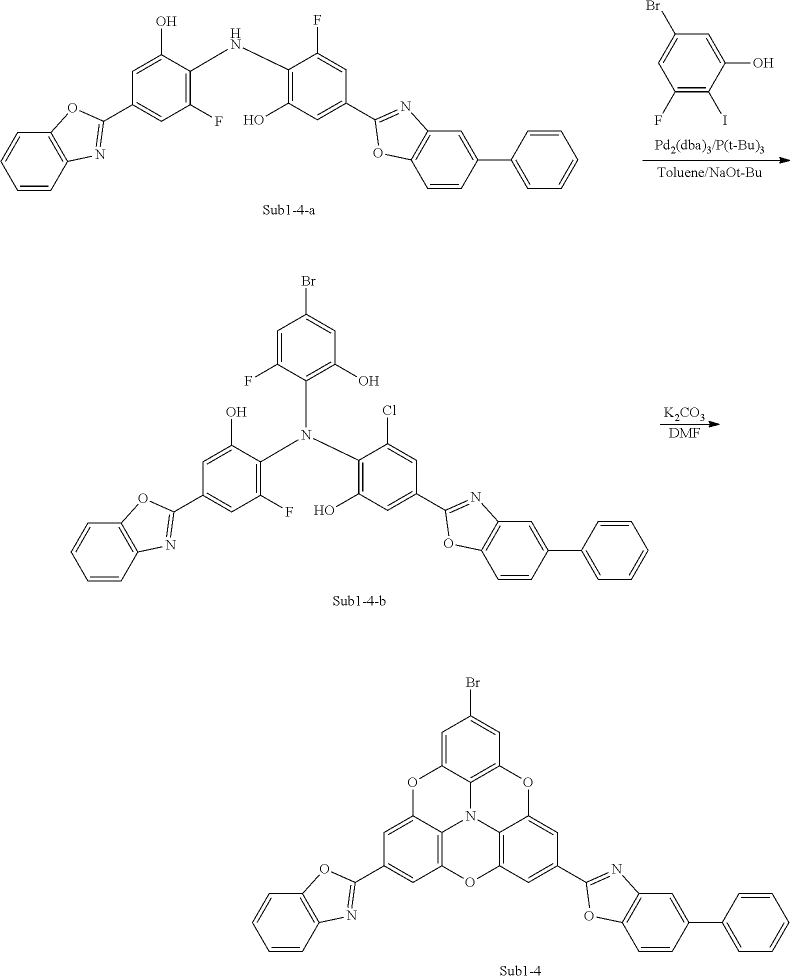

### (1) Synthesis of Sub1-4-a-1

2-amino-3-fluoro-5-iodophenol (20.0 g, 79.0 mmol), bis(pinacolato)diboron (22.1 g, 87.0 mmol), KOAc (23.3 g, 237 mmol), PdCl₂(dppf) (1.74 g, 2.37 mmol) were tested in the same manner as in Sub1-1-a-1 above to obtain 14.6 g of the product. (Yield 73%)

### (2) Synthesis of Sub1-4-a-2

After dissolving Sub1-4-a-1 (14.6 g, 57.7 mmol) obtained in the above synthesis in THF (290 mL) in a round-bottom flask, 2-iodobenzo[d]oxazole (14.1 g, 57.7 mmol), K₂CO₃ (23.9 g, 173 mmol), Pd(PPh₃)₄ (4.00 g, 3.46 mmol), water (145 mL) were added and the experiment was performed in the same manner as in Sub1-1-a-2 to obtain 11.6 g of a product. (Yield 82%)

### (3) Synthesis of Sub1-4-a-3

2-bromo-3-fluoro-5-iodophenol (20.0 g, 63.1 mmol), bis(pinacolato)diboron (17.6 g, 69.4 mmol), KOAc (18.6 g, 189 mmol), PdCl₂(dppf) (1.39 g, 1.89 mmol) were tested in the same manner as in Sub1-1-a-1 to obtain 13.6 g of the product. (Yield 68%)

### (4) Synthesis of Sub1-4-a-4

After dissolving Sub1-4-a-3 (13.6 g, 42.9 mmol) obtained in the above synthesis in THF (215 mL) in a round-bottom flask, 2-iodo-5-phenylbenzo[d]oxazole (13.8 g, 42.9 mmol), K₂CO₃ (17.8 g, 129 mmol), Pd(PPh₃)₄ (2.98 g, 2.57 mmol), water (107 mL) were added and the experiment was performed in the same manner as in Sub1-1-a-2 to obtain 14.0 g of a product. (Yield 85%)

### (5) Synthesis of Sub1-4-a

After dissolving Sub1-4-a-2 (8.9 g, 36.5 mmol) obtained in the above synthesis in Toluene (180 mL) in a round-bottom flask, Sub1-4-a-4 (14.0 g, 36.5 mmol) obtained in the above synthesis, Pd₂(dba)₃ (1.00 g, 1.09 mmol), P(t-Bu)₃ (0.44 g, 2.19 mmol), NaOt-Bu (7.0 g, 73.0 mmol) were added and tested in the same manner as in Sub1-1-a to obtain 15.0 g of the product. (yield 75%)

### (6) Synthesis of Sub1-4-b

After dissolving Sub1-4-a (15.0 g, 27.4 mmol) obtained in the above synthesis in Toluene (140 mL) in a round-bottom flask, 5-bromo-3-fluoro-2-iodophenol (9.6 g, 27.4 mmol), Pd₂(dba)₃ (0.75 g, 0.82 mmol), P(t-Bu)₃ (0.33 g, 1.64 mmol), NaOt-Bu (5.3 g, 54.7 mmol) were added, and the experiment was performed in the same manner as in Sub1-1-b, to obtain 15.8 g of a product. (yield 76%)

### (7) Synthesis of Sub1-4

After dissolving Sub1-4-b (15.8 g, 20.8 mmol) obtained in the above synthesis in DMF (208 mL) in a round bottom flask, K₂CO₃ (12.9 g, 93.6 mmol) was added and stirred for 12 hours. When the reaction is complete, extraction is performed with ethylacetate and water, and the organic layer is dried over MgSO₄ and concentrated. Thereafter, the resulting organic material was recrystallized using a silicagel column to obtain 5.9 g of a product. (Yield 42%)

### 3. Synthesis Example of Sub1-29

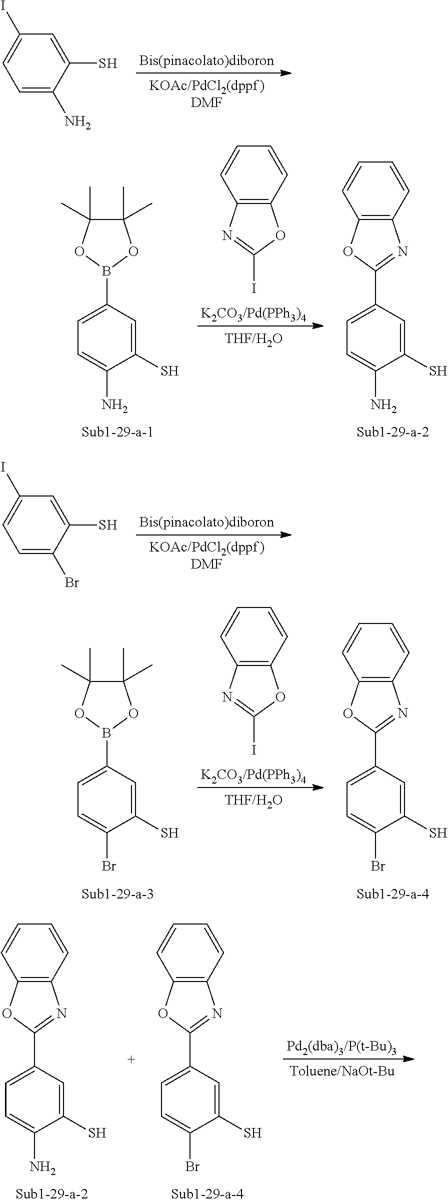

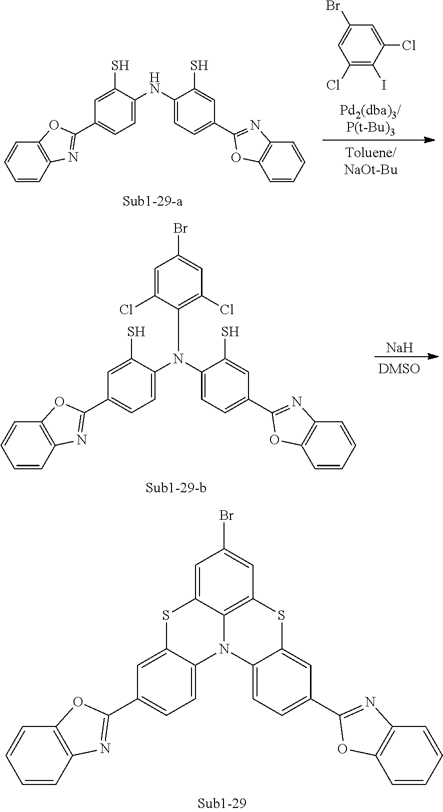

### (1) Synthesis of Sub1-29-a-1

2-amino-5-iodobenzenethiol (20.0 g, 79.7 mmol), bis(pinacolato)diboron (22.2 g, 87.6 mmol), KOAc (23.5 g, 239 mmol), PdCl₂(dppf) (1.75 g, 2.39 mmol) were tested in the same manner as in Sub1-1-a-1 above to obtain 14.8 g of the product. (Yield 74%)

### (2) Synthesis of Sub1-29-a-2

After dissolving Sub1-29-a-1 (14.8 g, 58.9 mmol) obtained in the above synthesis in THF (295 mL) in a round-bottom flask, 2-iodobenzo[d]oxazole (14.4 g, 58.9 mmol), K2CO3 (24.4 g, 177 mmol), Pd(PPh₃)₄ (4.09 g, 3.54 mmol), water (147 mL) were added, and the experiment was performed in the same manner as in Sub1-1-a-2, to obtain 11.4 g of a product. (Yield 80%)

### (3) Synthesis of Sub1-29-a-3

2-bromo-5-iodobenzenethiol (20.0 g, 63.5 mmol), bis(pinacolato)diboron (17.7 g, 69.8 mmol), KOAc (18.7 g, 191 mmol), PdCl₂(dppf) (1.39 g, 1.90 mmol) were tested in the same manner as in Sub1-1-a-1 above to obtain 14.2 g of the product. (Yield 71%)

### (4) Synthesis of Sub1-29-a-4

After dissolving Sub1-29-a-3 (14.2 g, 45.1 mmol) obtained in the above synthesis in THF (225 mL) in a round-bottom flask, 2-iodobenzo[d]oxazole (11.0 g, 45.1 mmol), K₂CO₃ (18.7 g, 135 mmol), Pd(PPh₃)₄ (3.13 g, 2.71 mmol), water (113 mL) were added, and the experiment was performed in the same manner as in Sub1-1-a-2, to obtain 11.3 g of a product. (Yield 82%)

### (5) Synthesis of Sub1-29-a

After dissolving Sub1-29-a-2 (9.0 g, 37.0 mmol) obtained in the above synthesis in Toluene (185 mL) in a round-bottom flask, Sub1-29-a-4 (11.3 g, 37.0 mmol) obtained in the above synthesis, Pd₂(dba)₃ (1.02 g, 1.11 mmol), P(t-Bu)₃ (0.45 g, 2.22 mmol), NaOt-Bu (7.1 g, 73.9 mmol) were added and tested in the same manner as in Sub1-1-a to obtain 12.3 g of a product. (Yield 71%)

### (6) Synthesis of Sub1-29-b

After dissolving Sub1-29-a (12.3 g, 26.2 mmol) obtained in the above synthesis in Toluene (131 mL) in a round bottom flask, 5-bromo-1,3-dichloro-2-iodobenzene (9.2 g, 26.2 mmol), Pd₂(dba)₃ (0.72 g, 0.79 mmol), P(t-Bu)₃ (0.32 g, 1.57 mmol), NaOt-Bu (5.0 g, 52.5 mmol) were added and the experiment was performed in the same manner as in Sub1-1-b, to obtain 13.4 g of a product. (Yield 74%)

### (7) Synthesis of Sub1-29

In a round bottom flask, NaH (90%) (2.07 g, 86.1 mmol), DMSO (194 mL) and Sub1-29-b (13.4 g, 19.4 mmol) obtained in the above synthesis were tested in the same manner as in Sub1-1 to obtain 7.4 g of product. (Yield 62%)

### 4. Synthesis Example of Sub1-47

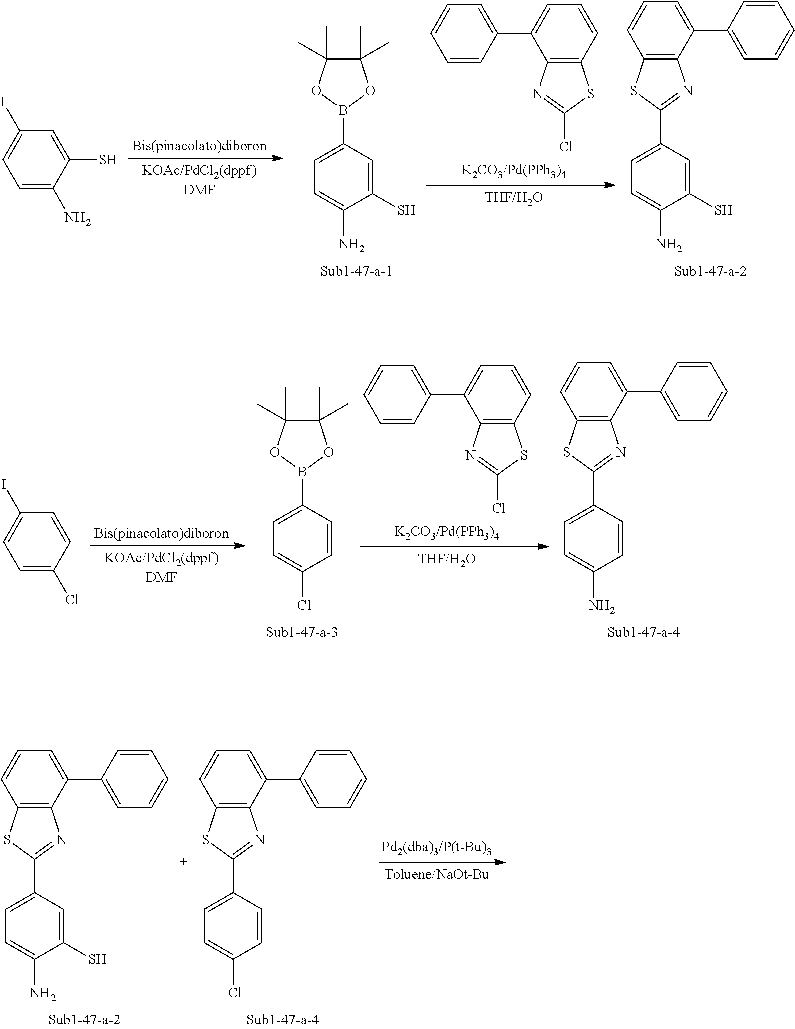

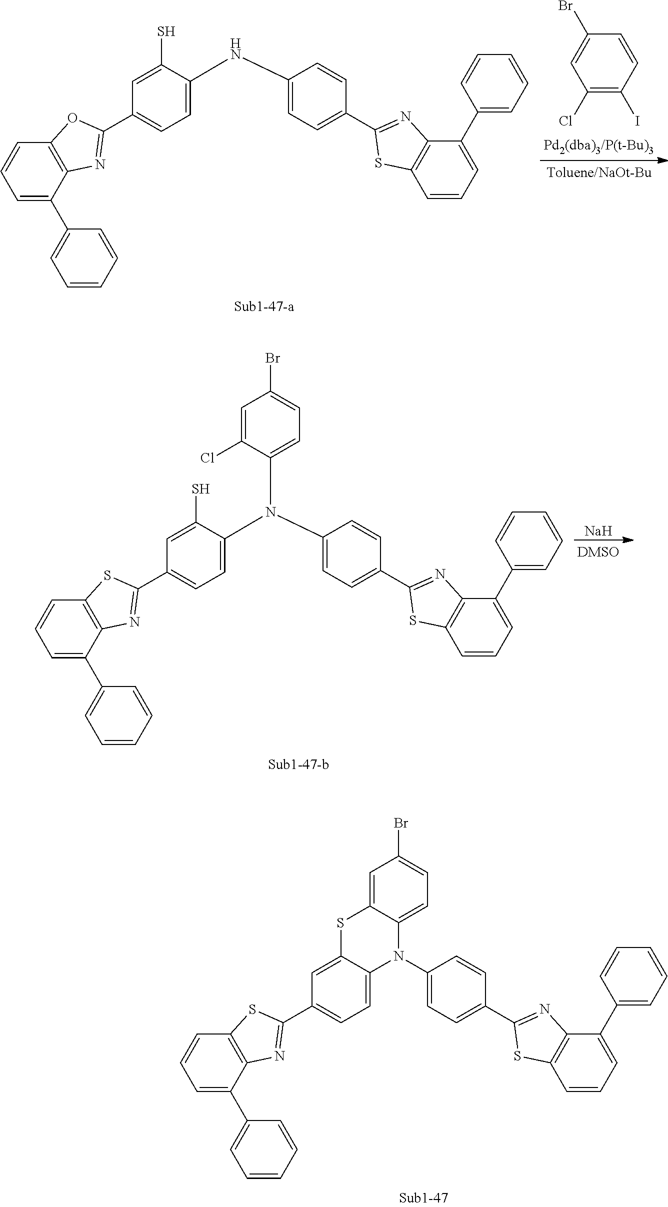

### (1) Synthesis of Sub1-47-a-1

2-amino-5-iodobenzenethiol (10.0 g, 39.8 mmol), bis(pinacolato)diboron (11.1 g, 43.8 mmol), KOAc (11.7 g, 120 mmol), PdCl₂(dppf) (0.87 g, 1.19 mmol) were tested in the same manner as in Sub1-1-a-1 above to obtain 7.3 g of the product. (Yield 73%)

### (2) Synthesis of Sub1-47-a-2

After dissolving Sub1-47-a-1 (7.3 g, 29.1 mmol) obtained in the above synthesis in THF (145 mL) in a round-bottom flask, 2-chloro-4-phenylbenzo[d]thiazole (7.1 g, 29.1 mmol), K₂CO₃ (12.1 g, 87.2 mmol), Pd(PPh₃)₄ (2.02 g, 1.74 mmol), water (73 mL) were added and tested in the same manner as in Sub1-1-a-2 to obtain 8.3 g of a product. (yield 85%)

### (3) Synthesis of Sub1-47-a-3

1-chloro-4-iodobenzene (10.0 g, 41.9 mmol), bis(pinacolato)diboron (11.7 g, 46.1 mmol), KOAc (12.3 g, 126 mmol), PdCl₂(dppf) (0.92 g, 1.26 mmol) were tested in the same manner as in Sub1-1-a-1 above to obtain 7.0 g of the product. (yield 70%)

### (4) Synthesis of Sub1-47-a-4

After dissolving Sub1-47-a-3 (7.0 g, 29.4 mmol) obtained in the above synthesis in THF (147 mL) in a round-bottom flask, 2-chloro-4-phenylbenzo[d]thiazole (7.2 g, 29.4 mmol), K₂CO₃ (12.2 g, 88.1 mmol), Pd(PPh₃)₄ (2.04 g, 1.76 mmol), water (73 mL) were added, and the same method as in Sub1-1-a-2 was performed to obtain 7.8 g of a product. (Yield 83%)

### (5) Synthesis of Sub1-47-a

After dissolving Sub1-47-a-2 (8.1 g, 24.4 mmol) obtained in the above synthesis in Toluene (122 mL) in a round-bottom flask, Sub1-47-a-4 (7.8 g, 24.4 mmol) obtained in the above synthesis, Pd₂(dba)₃ (0.67 g, 0.73 mmol), P(t-Bu)₃ (0.30 g, 1.46 mmol), NaOt-Bu (4.7 g, 48.7 mmol) were added and tested in the same manner as in Sub1-1-a to obtain 10.7 g of a product. (Yield 71%)

### (6) Synthesis of Sub1-47-b

After dissolving Sub1-47-a (10.7 g, 17.3 mmol) obtained in the above synthesis in Toluene (86 mL) in a round-bottom flask, 4-bromo-2-chloro-1-iodobenzene (5.5 g, 17.3 mmol), Pd₂(dba)₃ (0.48 g, 0.52 mmol), P(t-Bu)₃ (0.21 g, 1.04 mmol), NaOt-Bu (3.3 g, 34.6 mmol) were added and the experiment was performed in the same manner as in Sub1-1-b, to obtain 10.6 g of a product. (yield 76%)

### (7) Synthesis of Sub1-47

In a round bottom flask, NaH (90%) (0.70 g, 29.2 mmol), DMSO (131 mL), and Sub1-47-b (10.6 g, 13.1 mmol) obtained in the above synthesis were tested in the same manner as in Sub1-1. 8.2 g of product were obtained. (Yield 81%)

### 5. Synthesis Example of Sub1-50

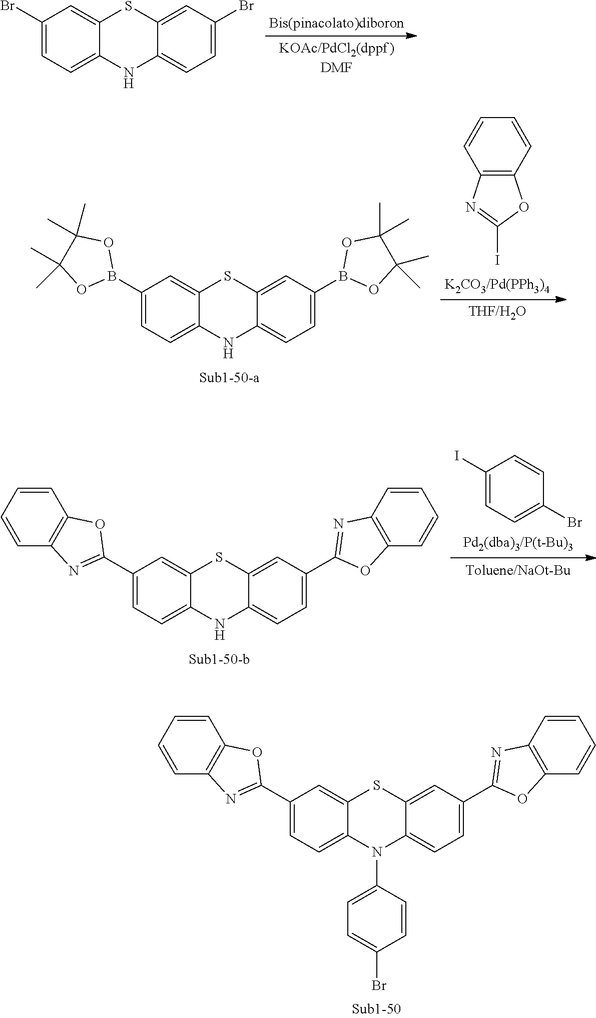

### (1) Synthesis of Sub1-50-a

3,7-dibromo-10H-phenothiazine (10.0 g, 28.0 mmol), bis(pinacolato)diboron (15.6 g, 61.6 mmol), KOAc (16.5 g, 168 mmol), PdCl₂(dppf) (1.23 g, 1.68 mmol) were tested in the same manner as in Sub1-1-a-1 above to obtain 7.1 g of the product. (Yield 56%)

### (2) Synthesis of Sub1-50-b

After dissolving Sub1-50-a (7.1 g, 15.7 mmol) obtained in the above synthesis in THF (78 mL) in a round-bottom flask, 2-iodobenzo[d]oxazole (8.5 g, 34.5 mmol), K₂CO₃ (6.5 g, 47.1 mmol), Pd(PPh₃)₄ (1.09 g, 0.94 mmol), water (39 mL) were added, and 4.6 g of a product was obtained by performing an experiment in the same manner as in Sub1-1-a-2. (Yield 67%)

### (3) Synthesis of Sub1-50

After dissolving Sub1-50-b (4.6 g, 10.5 mmol) obtained in the above synthesis in Toluene (53 mL) in a round-bottom flask, 1-bromo-4-iodobenzene (3.0 g, 10.5 mmol), Pd₂(dba)₃ (0.29 g, 0.32 mmol), P(t-Bu)₃ (0.13 g, 0.63 mmol), NaOt-Bu (2.0 g, 21.0 mmol) were added and an experiment was performed in the same manner as in Sub1-1-b to obtain 4.8 g of a product. (yield 78%)

Meanwhile, the compound belonging to Sub 1 may be a compound as follows, but is not limited thereto.

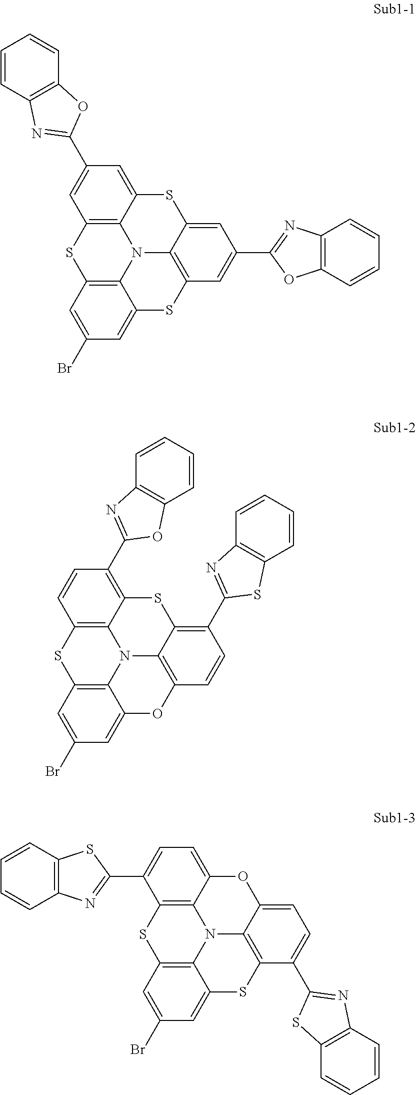 [SMILES: Brc1cc2c3c(c1)Sc1cc(-c4nc5ccccc5o4)cc4c1N3c1c(cc(-c3nc5ccccc5o3)cc1S4)S2 ; Brc1cc2c3c(c1)Sc1ccc(-c4nc5ccccc5o4)c4c1N3c1c(ccc(-c3nc5ccccc5s3)c1S4)O2 ; Brc1cc2c3c(c1)Sc1c(-c4nc5ccccc5s4)ccc4c1N3c1c(ccc(-c3nc5ccccc5s3)c1S2)O4]

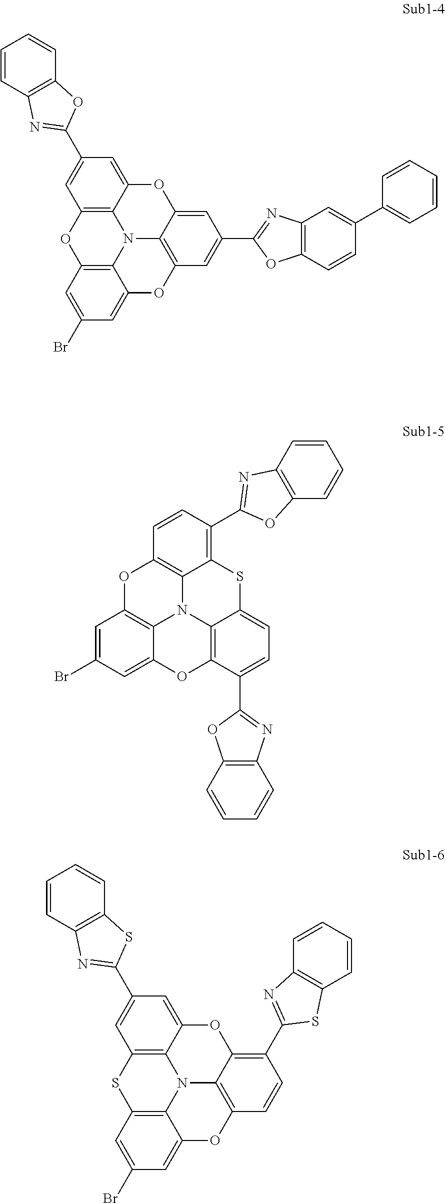 [SMILES: Brc1cc2c3c(c1)Oc1cc(-c4nc5cc(-c6ccccc6)ccc5o4)cc4c1N3c1c(cc(-c3nc5ccccc5o3)cc1O4)O2 ; Brc1cc2c3c(c1)Oc1c(-c4nc5ccccc5o4)ccc4c1N3c1c(ccc(-c3nc5ccccc5o3)c1S4)O2 ; Brc1cc2c3c(c1)Sc1cc(-c4nc5ccccc5s4)cc4c1N3c1c(ccc(-c3nc5ccccc5s3)c1O4)O2]

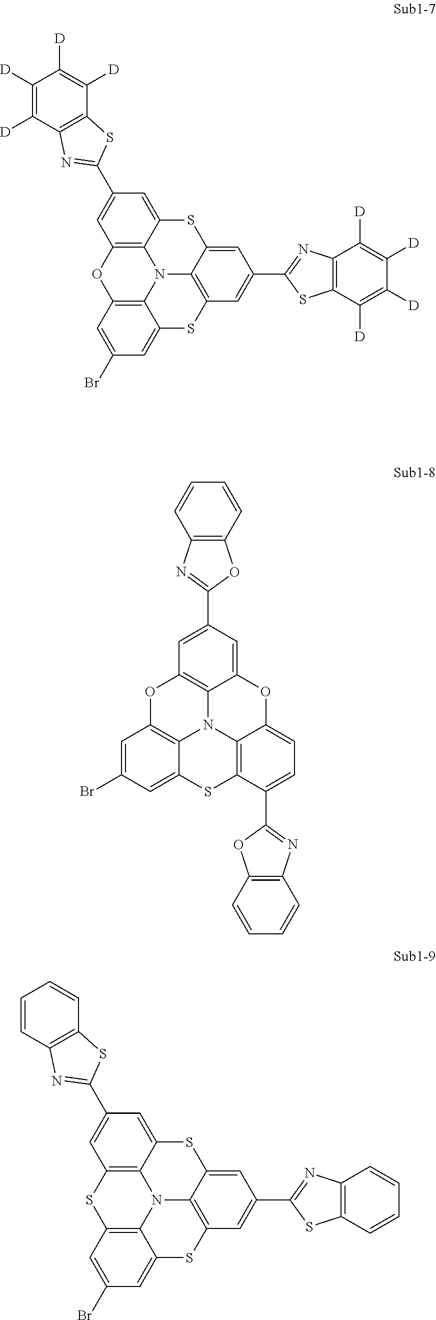 [SMILES: [2H]c1c([2H])c([2H])c2sc(-c3cc4c5c(c3)Sc3cc(-c6nc7c([2H])c([2H])c([2H])c([2H])c7s6)cc6c3N5c3c(cc(Br)cc3S6)O4)nc2c1[2H] ; Brc1cc2c3c(c1)Sc1c(-c4nc5ccccc5o4)ccc4c1N3c1c(cc(-c3nc5ccccc5o3)cc1O4)O2 ; Brc1cc2c3c(c1)Sc1cc(-c4nc5ccccc5s4)cc4c1N3c1c(cc(-c3nc5ccccc5s3)cc1S4)S2]

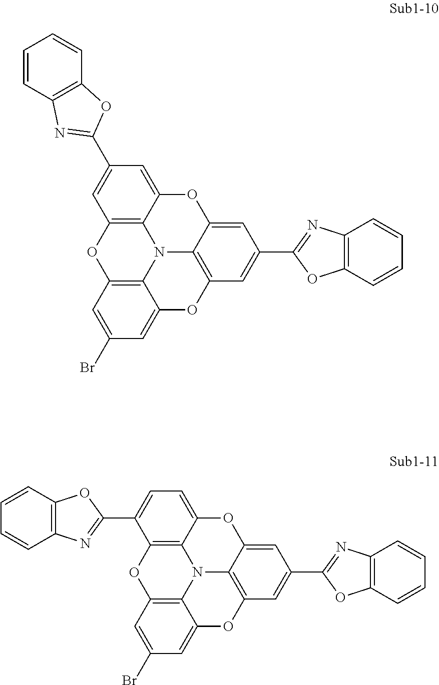 [SMILES: Brc1cc2c3c(c1)Oc1cc(-c4nc5ccccc5o4)cc4c1N3c1c(cc(-c3nc5ccccc5o3)cc1O4)O2 ; Brc1cc2c3c(c1)Oc1c(-c4nc5ccccc5o4)ccc4c1N3c1c(cc(-c3nc5ccccc5o3)cc1O4)O2]

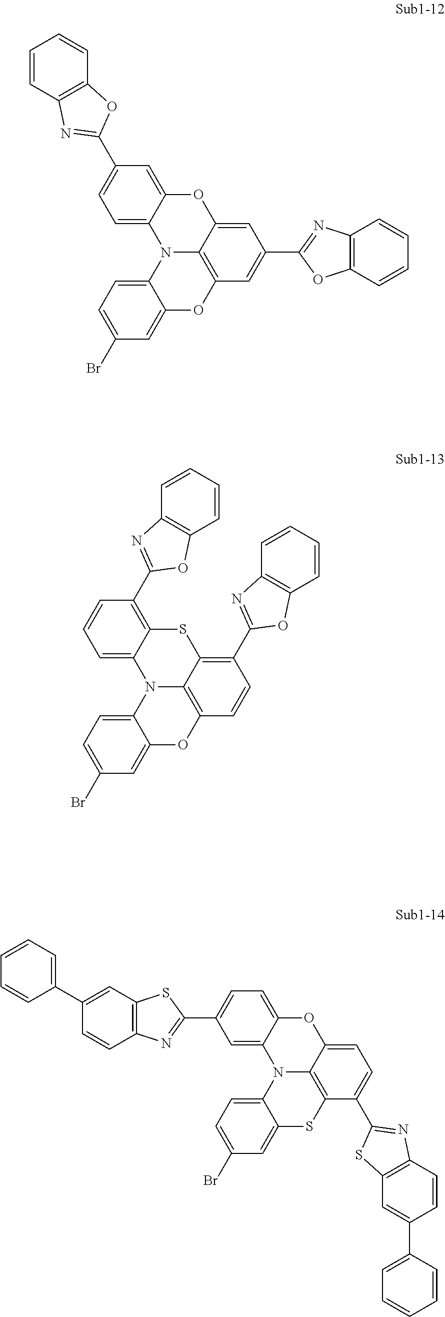 [SMILES: Brc1ccc2c(c1)Oc1cc(-c3nc4ccccc4o3)cc3c1N2c1ccc(-c2nc4ccccc4o2)cc1O3 ; Brc1ccc2c(c1)Oc1ccc(-c3nc4ccccc4o3)c3c1N2c1cccc(-c2nc4ccccc4o2)c1S3 ; Brc1ccc2c(c1)Sc1c(-c3nc4ccc(-c5ccccc5)cc4s3)ccc3c1N2c1cc(-c2nc4ccc(-c5ccccc5)cc4s2)ccc1O3]

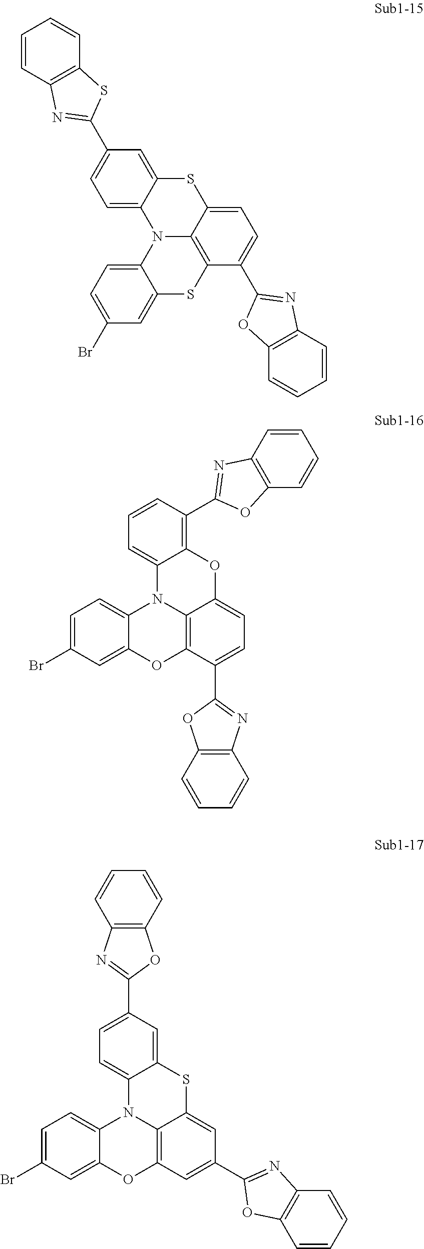

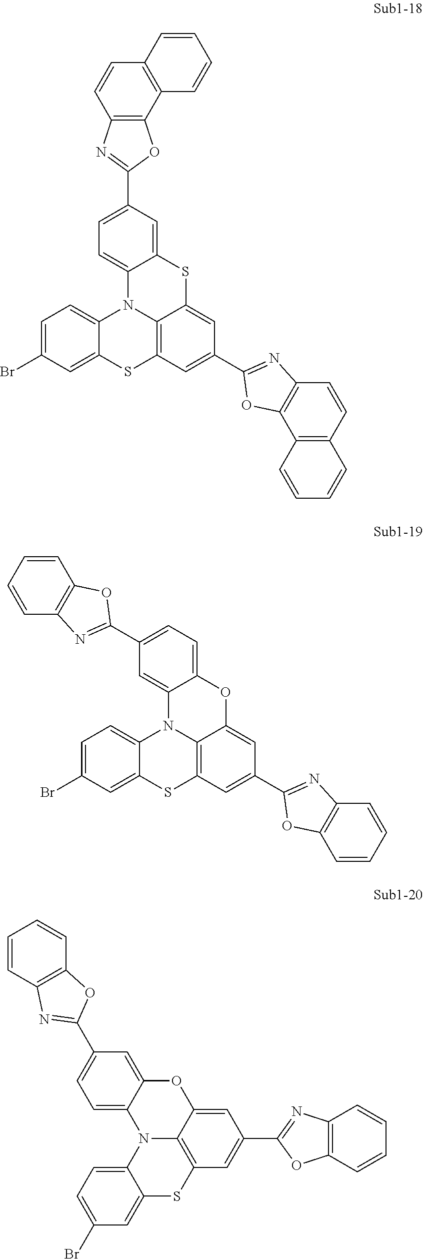 [SMILES: Brc1ccc2c(c1)Sc1cc(-c3nc4ccc5ccccc5c4o3)cc3c1N2c1ccc(-c2nc4ccc5ccccc5c4o2)cc1S3 ; Brc1ccc2c(c1)Sc1cc(-c3nc4ccccc4o3)cc3c1N2c1cc(-c2nc4ccccc4o2)ccc1O3 ; Brc1ccc2c(c1)Sc1cc(-c3nc4ccccc4o3)cc3c1N2c1ccc(-c2nc4ccccc4o2)cc1O3]

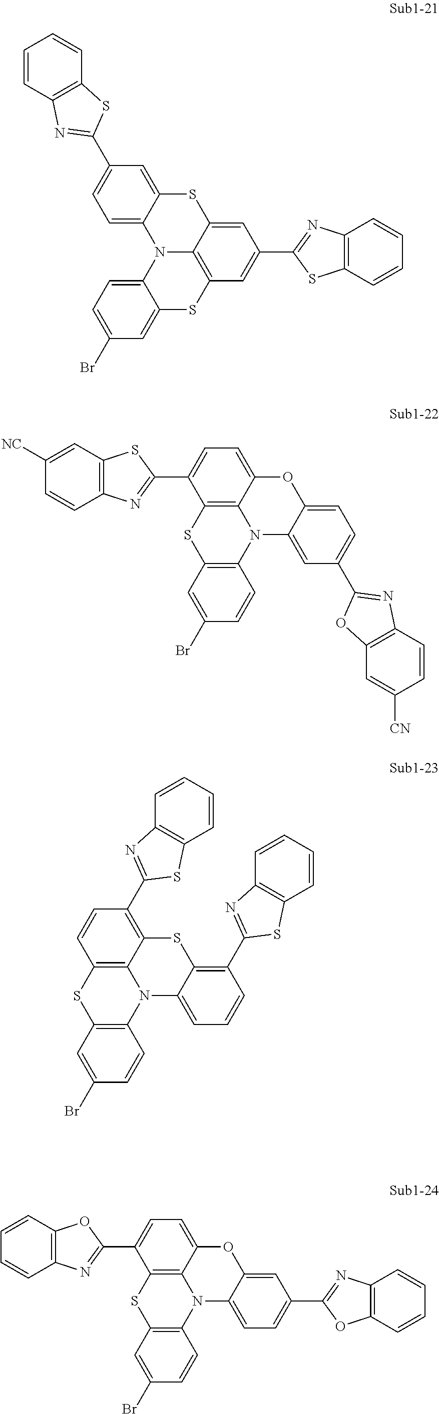 [SMILES: Brc1ccc2c(c1)Sc1cc(-c3nc4ccccc4s3)cc3c1N2c1ccc(-c2nc4ccccc4s2)cc1S3 ; N#Cc1ccc2nc(-c3ccc4c(c3)N3c5ccc(Br)cc5Sc5c(-c6nc7ccc(C#N)cc7s6)ccc(c53)O4)oc2c1 ; Brc1ccc2c(c1)Sc1ccc(-c3nc4ccccc4s3)c3c1N2c1cccc(-c2nc4ccccc4s2)c1S3 ; Brc1ccc2c(c1)Sc1c(-c3nc4ccccc4o3)ccc3c1N2c1ccc(-c2nc4ccccc4o2)cc1O3]

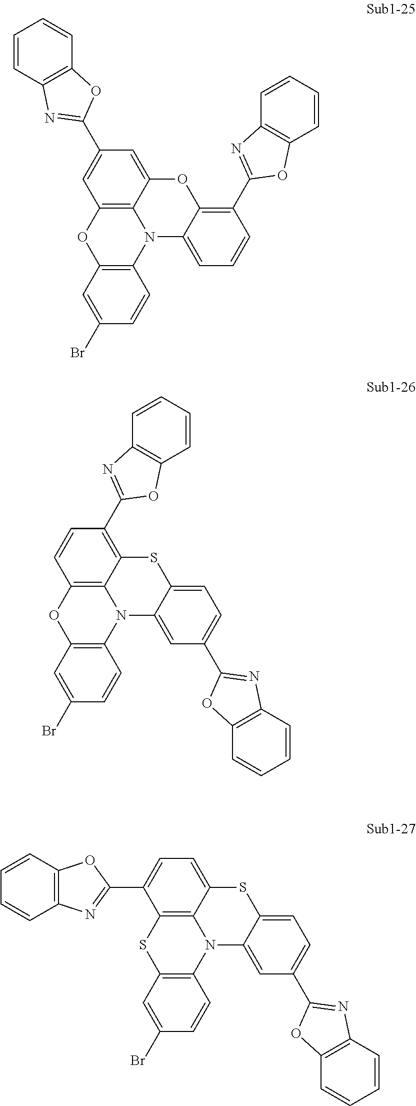 [SMILES: Brc1ccc2c(c1)Oc1cc(-c3nc4ccccc4o3)cc3c1N2c1cccc(-c2nc4ccccc4o2)c1O3 ; Brc1ccc2c(c1)Oc1ccc(-c3nc4ccccc4o3)c3c1N2c1cc(-c2nc4ccccc4o2)ccc1S3 ; Brc1ccc2c(c1)Sc1c(-c3nc4ccccc4o3)ccc3c1N2c1cc(-c2nc4ccccc4o2)ccc1S3]

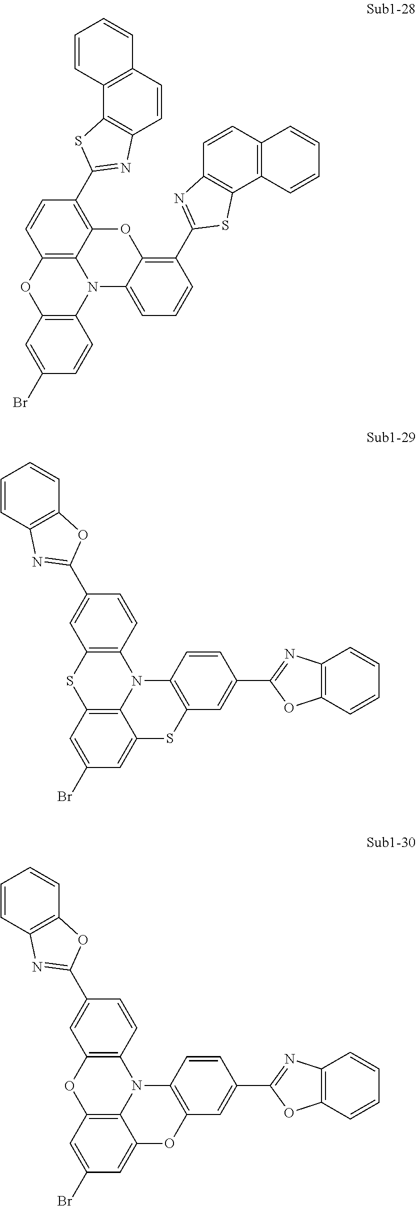 [SMILES: Brc1ccc2c(c1)Oc1ccc(-c3nc4ccc5ccccc5c4s3)c3c1N2c1cccc(-c2nc4ccc5ccccc5c4s2)c1O3 ; Brc1cc2c3c(c1)Sc1cc(-c4nc5ccccc5o4)ccc1N3c1ccc(-c3nc4ccccc4o3)cc1S2 ; Brc1cc2c3c(c1)Oc1cc(-c4nc5ccccc5o4)ccc1N3c1ccc(-c3nc4ccccc4o3)cc1O2]

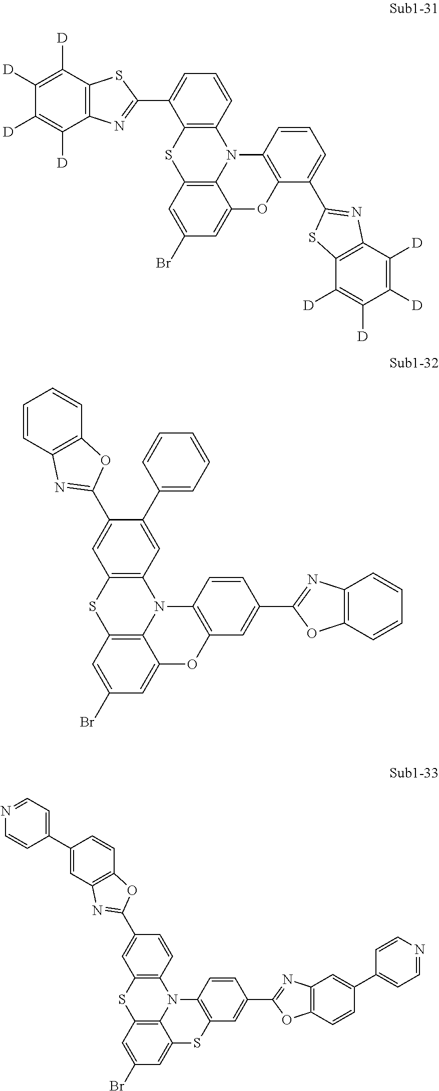 [SMILES: [2H]c1c([2H])c([2H])c2sc(-c3cccc4c3Oc3cc(Br)cc5c3N4c3cccc(-c4nc6c([2H])c([2H])c([2H])c([2H])c6s4)c3S5)nc2c1[2H] ; Brc1cc2c3c(c1)Sc1cc(-c4nc5ccccc5o4)c(-c4ccccc4)cc1N3c1ccc(-c3nc4ccccc4o3)cc1O2 ; Brc1cc2c3c(c1)Sc1cc(-c4nc5cc(-c6ccncc6)ccc5o4)ccc1N3c1ccc(-c3nc4cc(-c5ccncc5)ccc4o3)cc1S2]

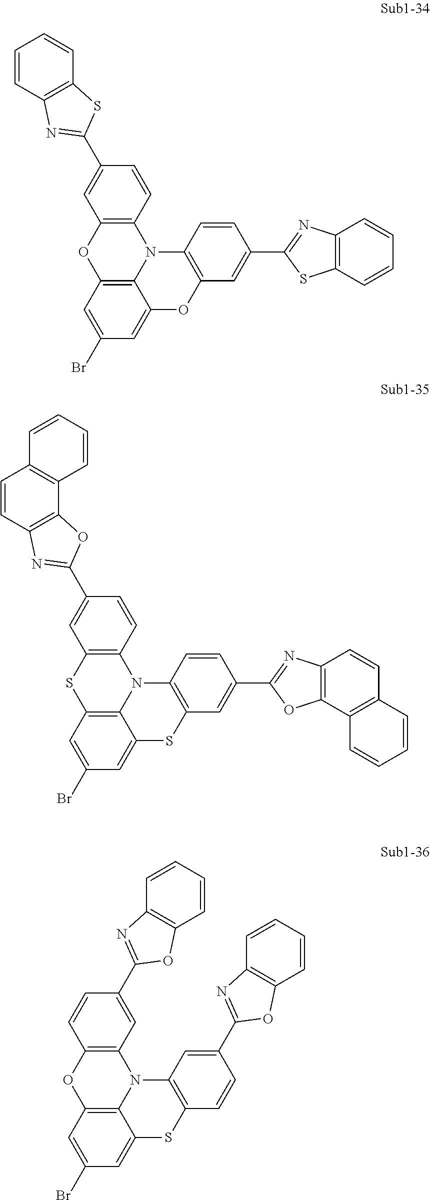 [SMILES: Brc1cc2c3c(c1)Oc1cc(-c4nc5ccccc5s4)ccc1N3c1ccc(-c3nc4ccccc4s3)cc1O2 ; Brc1cc2c3c(c1)Sc1cc(-c4nc5ccc6ccccc6c5o4)ccc1N3c1ccc(-c3nc4ccc5ccccc5c4o3)cc1S2 ; Brc1cc2c3c(c1)Sc1ccc(-c4nc5ccccc5o4)cc1N3c1cc(-c3nc4ccccc4o3)ccc1O2]

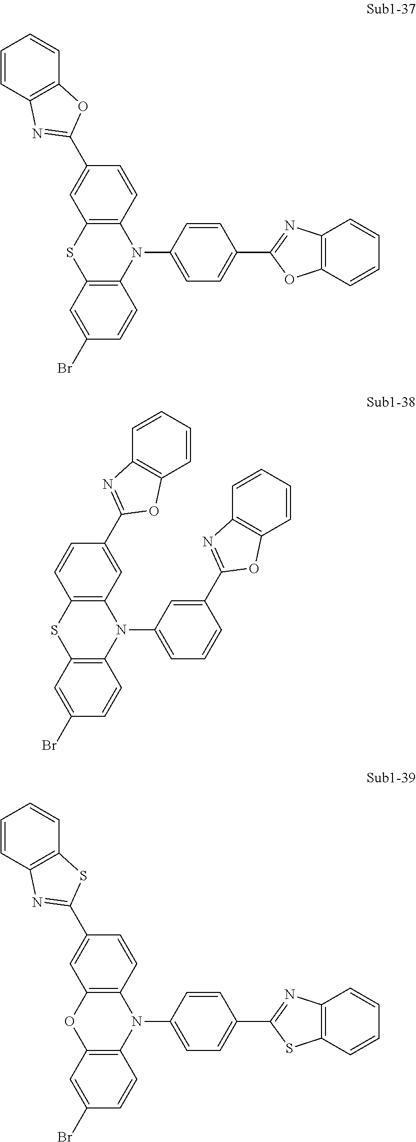 [SMILES: Brc1ccc2c(c1)Sc1cc(-c3nc4ccccc4o3)ccc1N2c1ccc(-c2nc3ccccc3o2)cc1 ; Brc1ccc2c(c1)Sc1ccc(-c3nc4ccccc4o3)cc1N2c1cccc(-c2nc3ccccc3o2)c1 ; Brc1ccc2c(c1)Oc1cc(-c3nc4ccccc4s3)ccc1N2c1ccc(-c2nc3ccccc3s2)cc1]

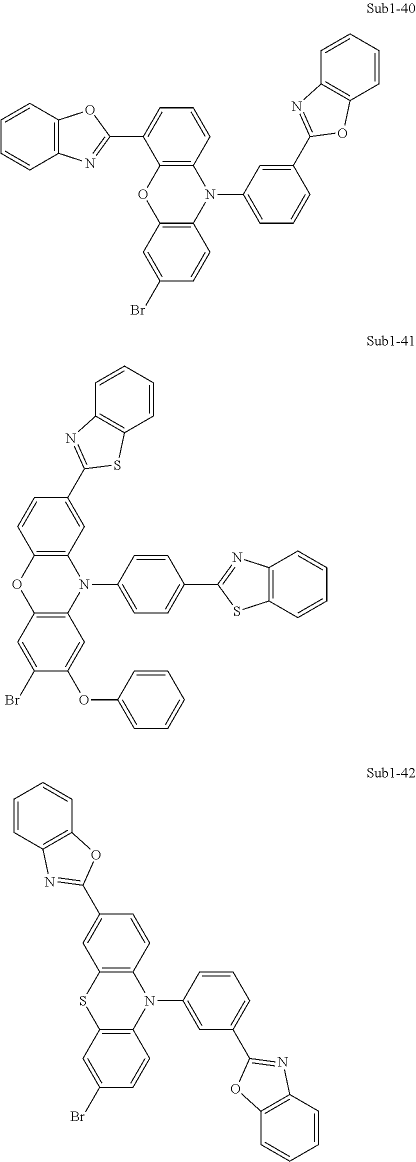 [SMILES: Brc1ccc2c(c1)Oc1c(-c3nc4ccccc4o3)cccc1N2c1cccc(-c2nc3ccccc3o2)c1 ; Brc1cc2c(cc1Oc1ccccc1)N(c1ccc(-c3nc4ccccc4s3)cc1)c1cc(-c3nc4ccccc4s3)ccc1O2 ; Brc1ccc2c(c1)Sc1cc(-c3nc4ccccc4o3)ccc1N2c1cccc(-c2nc3ccccc3o2)c1]

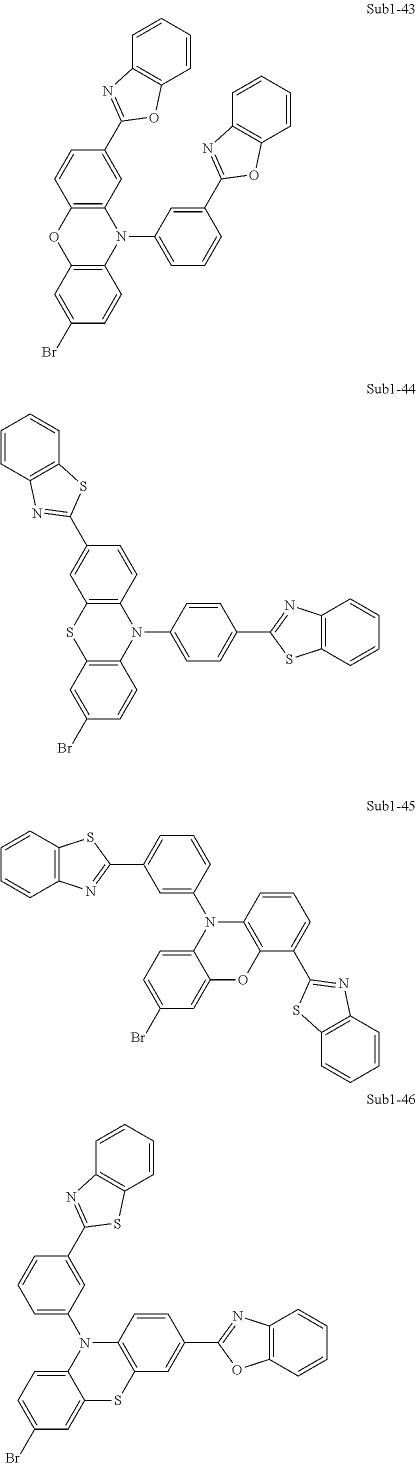 [SMILES: Brc1ccc2c(c1)Oc1ccc(-c3nc4ccccc4o3)cc1N2c1cccc(-c2nc3ccccc3o2)c1 ; Brc1ccc2c(c1)Sc1cc(-c3nc4ccccc4s3)ccc1N2c1ccc(-c2nc3ccccc3s2)cc1 ; Brc1ccc2c(c1)Oc1c(-c3nc4ccccc4s3)cccc1N2c1cccc(-c2nc3ccccc3s2)c1 ; Brc1ccc2c(c1)Sc1cc(-c3nc4ccccc4o3)ccc1N2c1cccc(-c2nc3ccccc3s2)c1]

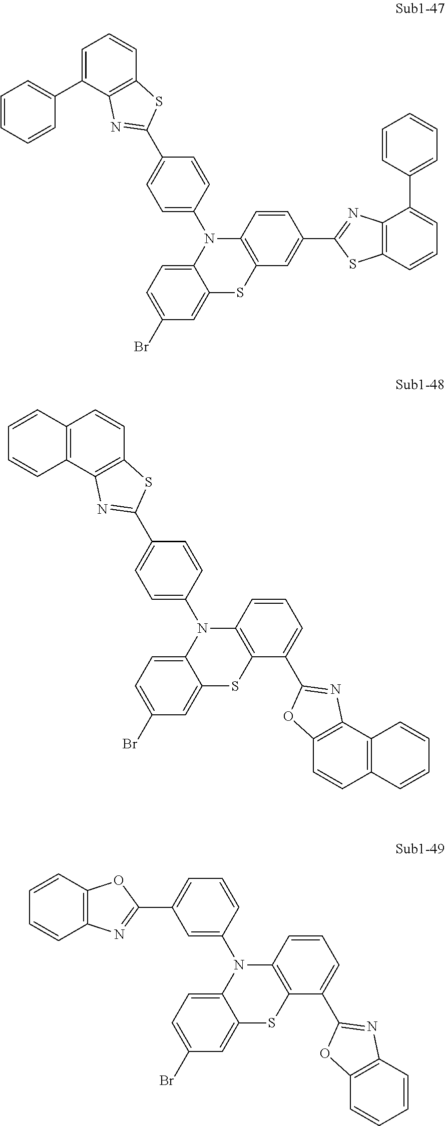 [SMILES: Brc1ccc2c(c1)Sc1cc(-c3nc4c(-c5ccccc5)cccc4s3)ccc1N2c1ccc(-c2nc3c(-c4ccccc4)cccc3s2)cc1 ; Brc1ccc2c(c1)Sc1c(-c3nc4c(ccc5ccccc54)o3)cccc1N2c1ccc(-c2nc3c(ccc4ccccc43)s2)cc1 ; Brc1ccc2c(c1)Sc1c(-c3nc4ccccc4o3)cccc1N2c1cccc(-c2nc3ccccc3o2)c1]

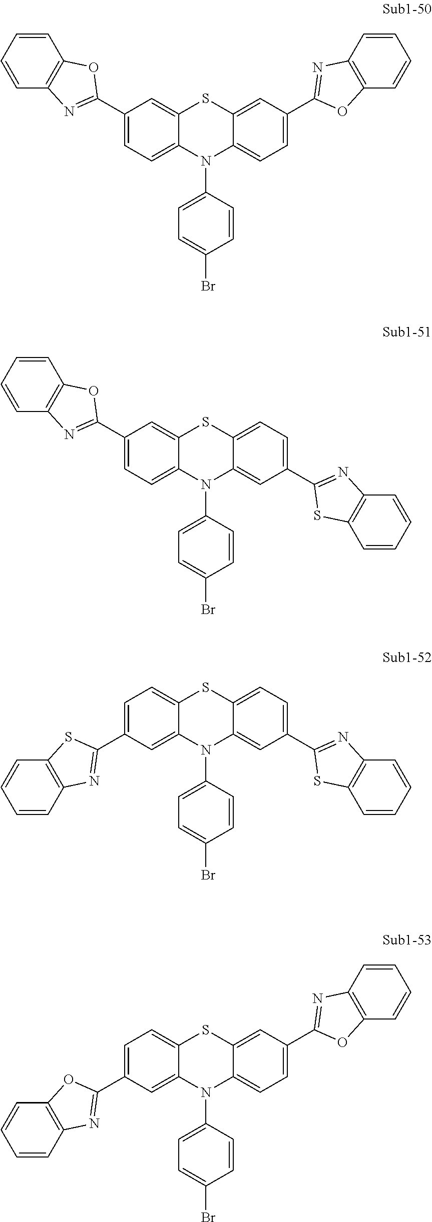 [SMILES: Brc1ccc(N2c3ccc(-c4nc5ccccc5o4)cc3Sc3cc(-c4nc5ccccc5o4)ccc32)cc1 ; Brc1ccc(N2c3ccc(-c4nc5ccccc5o4)cc3Sc3ccc(-c4nc5ccccc5s4)cc32)cc1 ; Brc1ccc(N2c3cc(-c4nc5ccccc5s4)ccc3Sc3ccc(-c4nc5ccccc5s4)cc32)cc1 ; Brc1ccc(N2c3ccc(-c4nc5ccccc5o4)cc3Sc3ccc(-c4nc5ccccc5o4)cc32)cc1]

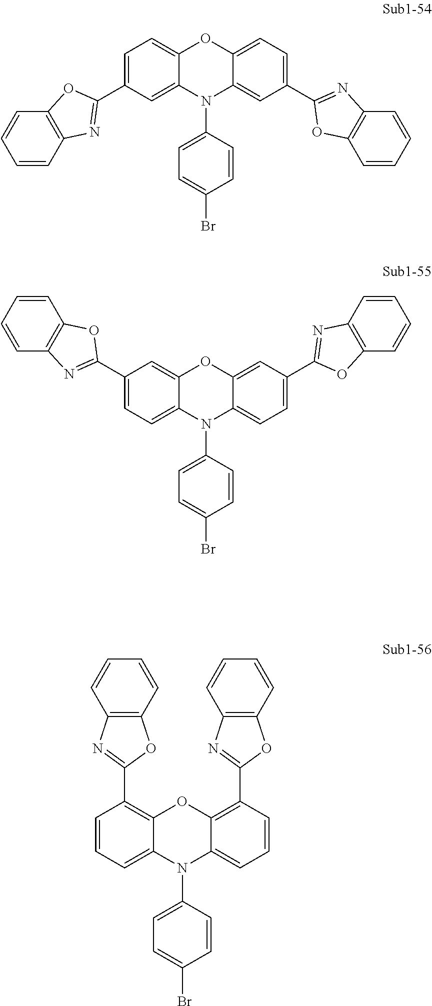 [SMILES: Brc1ccc(N2c3cc(-c4nc5ccccc5o4)ccc3Oc3ccc(-c4nc5ccccc5o4)cc32)cc1 ; Brc1ccc(N2c3ccc(-c4nc5ccccc5o4)cc3Oc3cc(-c4nc5ccccc5o4)ccc32)cc1 ; Brc1ccc(N2c3cccc(-c4nc5ccccc5o4)c3Oc3c(-c4nc5ccccc5o4)cccc32)cc1]

Table 1 below shows the FD-MS (Field Desorption-Mass Spectrometry) values of the compounds belonging to Sub 1.

TABLE 1

| compound | FD-MS | compound | FD-MS |
|---|---|---|---|
| Sub1-1 | m/z = 646.94(C₃₂H₁₄BrN₃O₂S₃ = 648.57) | Sub1-2 | m/z = 646.94(C₃₂H₁₄BrN₃O₂S₃ = 648.57) |
| Sub1-3 | m/z = 662.92(C₃₂H₁₄BrN₃OS₄ = 664.63) | Sub1-4 | m/z = 675.04(C₃₈H₁₈BrN₃O₅ = 676.48) |
| Sub1-5 | m/z = 614.99(C₃₂H₁₄BrN₃O₄S = 616.45) | Sub1-6 | m/z = 646.94(C₃₂H₁₄BrN₃O₂S₃ = 648.57) |
| Sub1-7 | m/z = 670.97(C₃₂H₆D₈BrN₃OS₄ = 672.68) | Sub1-8 | m/z = 614.99(C₃₂H₁₄BrN₃O₄S = 616.45) |
| Sub1-9 | m/z = 678.9(C₃₂H₁₄BrN₃S₅ = 680.69) | Sub1-10 | m/z = 599.01(C₃₂H₁₄BrN₃O₅ = 600.38) |
| Sub1-11 | m/z = 599.01(C₃₂H₁₄BrN₃O₅ = 600.38) | Sub1-12 | m/z = 585.03(C₃₂H₁₆BrN₃O₄ = 586.4) |
| Sub1-13 | m/z = 601.01(C₃₂H₁₆BrN₃O₃S = 602.46) | Sub1-14 | m/z = 785.03(C₄₄H₂₄BrN₃OS₃ = 786.78) |
| Sub1-15 | m/z = 632.96(C₃₂H₁₆BrN₃OS₃ = 634.58) | Sub1-16 | m/z = 585.03(C₃₂H₁₆BrN₃O₄ = 586.4) |
| Sub1-17 | m/z = 601.01(C₃₂H₁₆BrN₃O₃S = 602.46) | Sub1-18 | m/z = 601.01(C₃₂H₁₆BrN₃O₃S = 602.46) |
| Sub1-19 | m/z = 601.01(C₃₂H₁₆BrN₃O₃S = 602.46) | Sub1-20 | m/z = 601.01(C₃₂H₁₆BrN₃O₃S = 602.46) |
| Sub1-21 | m/z = 648.94(C₃₂H₁₆BrN₃S₄ = 650.65) | Sub1-22 | m/z = 648.94(C₃₂H₁₆BrN₃S₄ = 650.65) |
| Sub1-23 | m/z = 648.94(C₃₂H₁₆BrN₃S₄ = 650.65) | Sub1-24 | m/z = 616.99(C₃₂H₁₆BrN₃O₂S₂ = 618.52) |
| Sub1-25 | m/z = 585.03(C₃₂H₁₆BrN₃O₄ = 586.4) | Sub1-26 | m/z = 601.01(C₃₂H₁₆BrN₃O₃S = 602.46) |
| Sub1-27 | m/z = 616.99(C₃₂H₁₆BrN₃O₂S₂ = 618.52) | Sub1-28 | m/z = 717.02(C₄₀H₂₀BrN₃O₂S₂ = 718.64) |
| Sub1-29 | m/z = 616.99(C₃₂H₁₆BrN₃O₂S₂ = 618.52) | Sub1-30 | m/z = 585.03(C₃₂H₁₆BrN₃O₄ = 586.4) |
| Sub1-31 | m/z = 641.01(C₃₂H₈D₈BrN₃OS₃ = 642.63) | Sub1-32 | m/z = 677.04(C₃₈H₂₀BrN₃O₃S = 678.56) |
| Sub1-33 | m/z = 755.06(C₄₂H₂₂BrN₅O₃S = 756.63) | Sub1-34 | m/z = 616.99(C₃₂H₁₆BrN₃O₂S₂ = 618.52) |
| Sub1-35 | m/z = 717.02(C₄₀H₂₈BrN₃O₂S₂ = 718.64) | Sub1-36 | m/z = 601.01(C₃₂H₁₆BrN₃O₃S = 602.46) |
| Sub1-37 | m/z = 587.03(C₃₂H₁₈BrN₃O₂S = 588.48) | Sub1-38 | m/z = 587.03(C₃₂H₁₈BrN₃O₂S = 588.48) |
| Sub1-39 | m/z = 603.01(C₃₂H₁₈BrN₃OS₂ = 604.54) | Sub1-40 | m/z = 571.05(C₃₂H₁₈BrN₃O₃ = 572.42) |
| Sub1-41 | m/z = 695.03(C₃₈H₂₂BrN₃O₂S₂ = 696.64) | Sub1-42 | m/z = 587.03(C₃₂H₁₈BrN₃O₂S = 588.48) |
| Sub1-43 | m/z = 571.05(C₃₂H₁₈BrN₃O₃ = 572.42) | Sub1-44 | m/z = 618.98(C₃₂H₁₈BrN₃S₃ = 620.6) |
| Sub1-45 | m/z = 603.01(C₃₂H₁₈BrN₃OS₂ = 604.54) | Sub1-46 | m/z = 603.01(C₃₂H₁₈BrN₃OS₂ = 604.54) |
| Sub1-47 | m/z = 771.05(C₄₄H₂₆BrN₃S₃ = 772.80) | Sub1-48 | m/z = 671.08(C₄₀H₂₂BrN₃O₃ = 672.54) |
| Sub1-49 | m/z = 587.03(C₃₂H₁₈BrN₃O₂S = 588.48) | Sub1-50 | m/z = 587.03(C₃₂H₁₈BrN₃O₂S = 588.48) |
| Sub1-51 | m/z = 587.03(C₃₂H₁₈BrN₃O₂S = 588.48) | Sub1-52 | m/z = 618.98(C₃₂H₁₈BrN₃S₃ = 620.6) |
| Sub1-53 | m/z = 587.03(C₃₂H₁₈BrN₃O₂S = 588.48) | Sub1-54 | m/z = 571.05(C₃₂H₁₈BrN₃O₃ = 572.42) |
| Sub1-55 | m/z = 571.05(C₃₂H₁₈BrN₃O₃ = 572.42) | Sub1-56 | m/z = 571.05 (C₃₂H₁₈BrN₃O₃ = 572.42) |

### II. Synthesis of Final Product

### 1. Synthesis Example of P-1

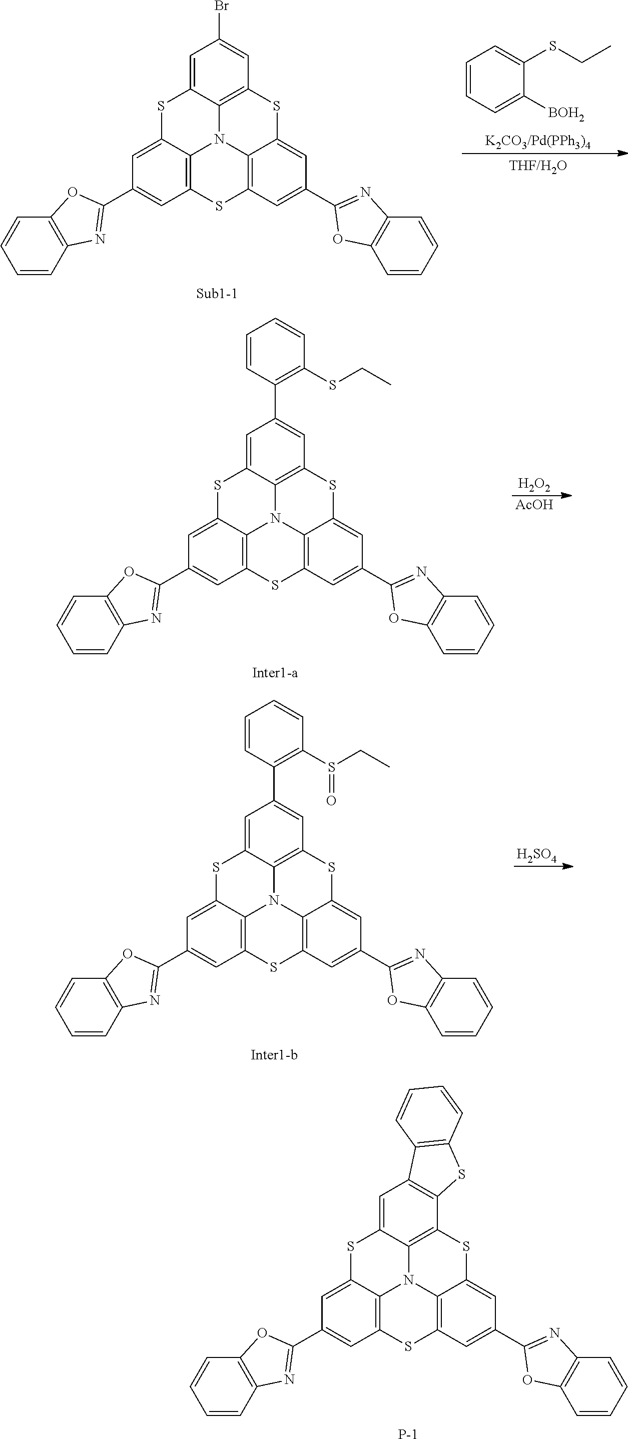

### (1) Synthesis of Inter1-a

After dissolving Sub1-1 (4.0 g, 6.2 mmol) obtained in the above synthesis in THF (30 mL) in a round-bottom flask, 2-Methylsulfanylphenylboronic acid (1.0 g, 6.2 mmol), Pd(PPh₃)₄ (0.43 g, 0.37 mmol), K₂CO₃ (2.6 g, 18.5 mmol), water

(15 mL) were added and stirred at 80° C. When the reaction is complete, the mixture is extracted with CH₂Cl₂ and water, and the organic layer is dried over MgSO₄ and concentrated. Thereafter, the resulting organic material was recrystallized using silicagel column to obtain 3.6 g of a product. (Yield 82%)

### (2) Synthesis of Inter1-b

To Inter1-a (3.6 g, 5.1 mmol) obtained in the above synthesis, acetic acid (20 mL) was added, 35% hydrogen peroxide (H₂O₂) (1.44 mL) was added, and the mixture was stirred at room temperature. When the reaction is complete, the reaction is neutralized with an aqueous NaOH solution, extracted with ethylacetate and water, and the organic layer is dried over MgSO₄ and concentrated. Then, the resulting organic material was recrystallized by sillicagel column to obtain 3.4 g of a product. (yield 93%)

### (3) Synthesis of P-1

Sulfuric acid (H₂SO₄) (10.2 mL) was added to Inter1-b (3.4 g, 4.7 mmol) obtained in the above synthesis and stirred at room temperature. After completion of the reaction, neutralized with aqueous NaOH solution, extracted with methylene chloride and water, the organic layer is dried over MgSO₄ and concentrated. Thereafter, the resulting organic material was recrystallized by sillicagel column to obtain 2.8 g of a product. (Yield 88%)

### 2. Synthesis of P-4

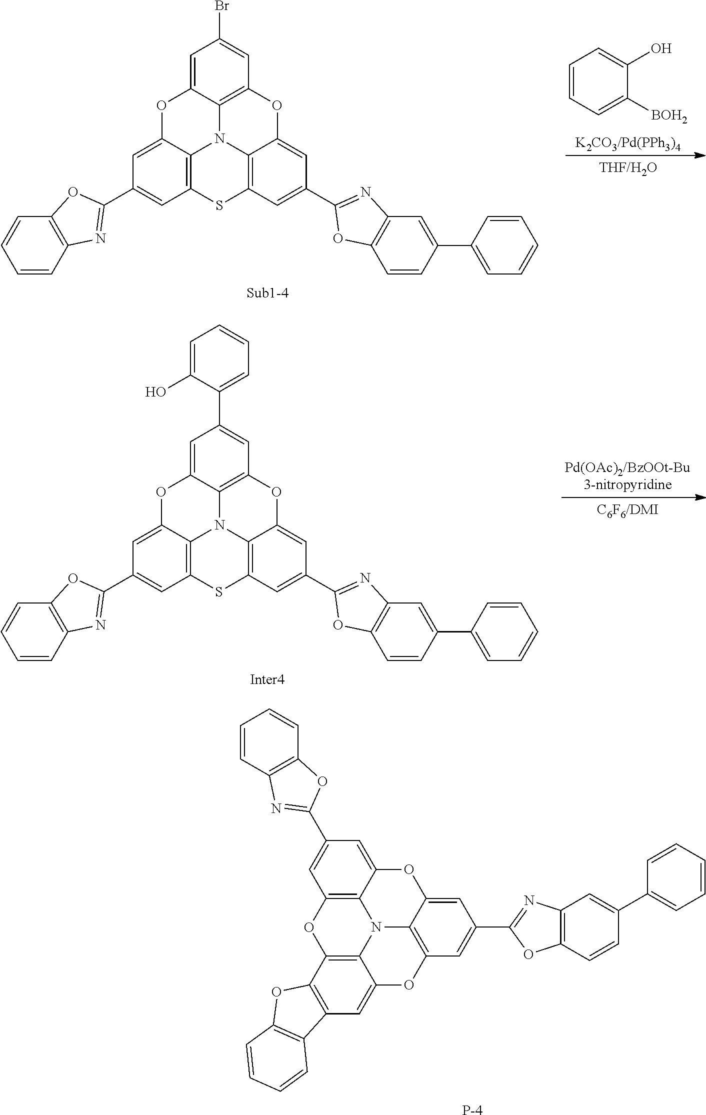

### (1) Synthesis of Inter4

After dissolving Sub1-4 (5.5 g, 8.1 mmol) obtained in the above synthesis in THF (40 mL) in a round-bottom flask, 2-Hydroxyphenylboronic acid (1.0 g, 8.1 mmol), K₂CO₃ (3.4 g, 24.4 mmol), Pd(PPh₃)₄ (0.56 g, 0.49 mmol), water (20 mL) were added, and 4.5 g of the product was obtained by performing an experiment in the same manner as for Inter1-a. (yield 80%)

### (2) Synthesis of P-4

Pd(OAc)₂ (0.07 g, 0.33 mmol), 3-nitropyridine (0.04 g, 0.33 mmol), BzOOtBu (tert-butyl peroxybenzoate) (2.5 g, 13.0 mmol), C₆F₆ (hexafluorobenzene) (10 mL), DMI (N,N′-dimethylimidazolidinone) (7 mL) were added to Inter4 (4.5 g, 6.5 mmol) obtained in the above synthesis and refluxed at 90° C. for 3 hours.

When the reaction is complete, extraction is performed with ethylacetate and water, and the organic layer is dried over MgSO₄ and concentrated. Thereafter, the resulting organic material was recrystallized by sillicagel column to obtain 2.8 g of a product. (Yield 62%)

### 3. Synthesis Example of P=37

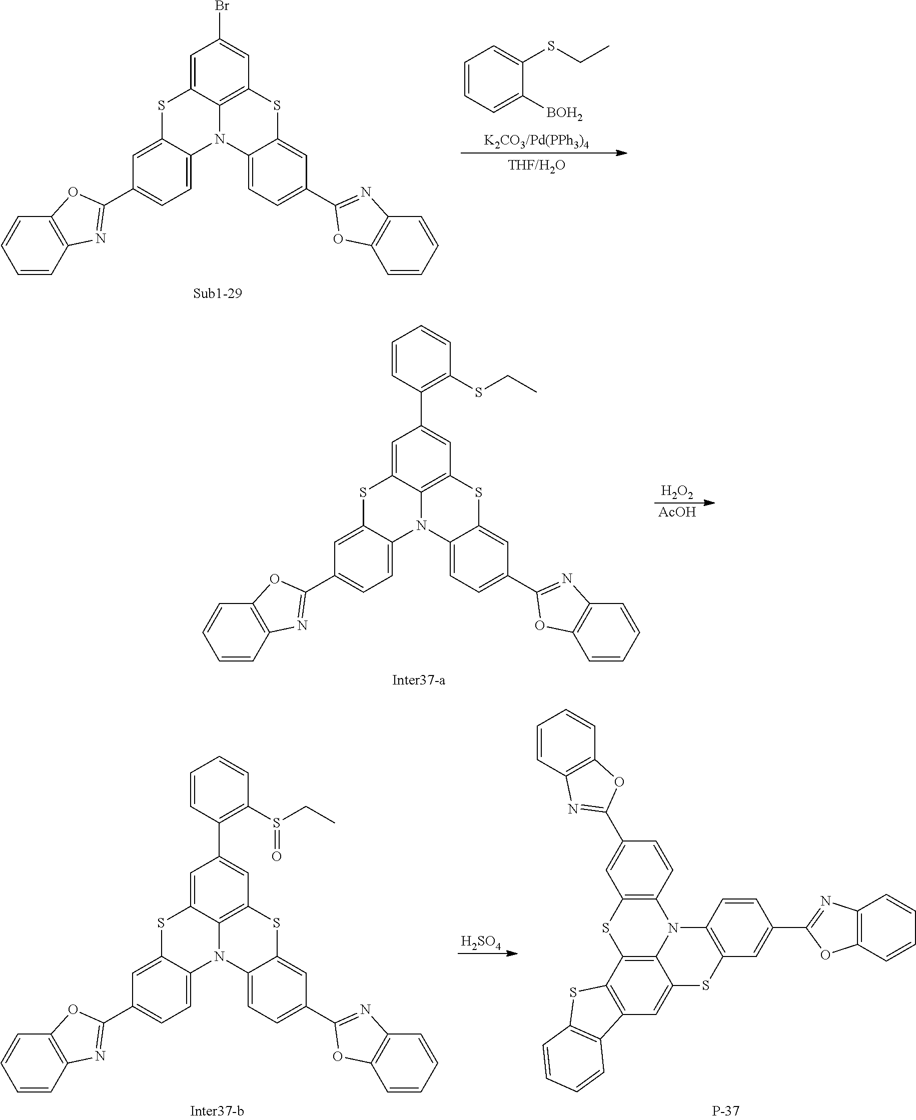

### (1) Synthesis of Inter37-a

After dissolving Sub1-29 (4.0 g, 6.5 mmol) obtained in the above synthesis in THF (32 mL) in a round-bottom flask, 2-Methylsulfanylphenylboronic acid (1.1 g, 6.5 mmol), K₂CO₃ (2.7 g, 19.4 mmol), Pd(PPh₃)₄ (0.45 g, 0.39 mmol), water (16 mL) were added and the experiment was performed in the same manner as for Inter1-a, to obtain 3.5 g of a product. (Yield 81%)

### (2) Synthesis of Inter37-b

Acetic acid (21 mL) and 35% hydrogen peroxide (H₂O₂) (1.50 mL) were added to Inter37-a (3.5 g, 5.2 mmol) obtained in the above synthesis, and the same procedure as for Inter1-b was performed to obtain 3.3 g of the product. (Yield 91%)

### (3) Synthesis of P-37

Sulfuric acid (H₂SO₄) (9.9 mL) was added to Inter37-b (3.3 g, 4.8 mmol) obtained in the above synthesis, and an experiment was performed in the same manner as in P-1 to obtain 2.8 g of a product. (yield 90%)

### 4. Synthesis Example of P-65

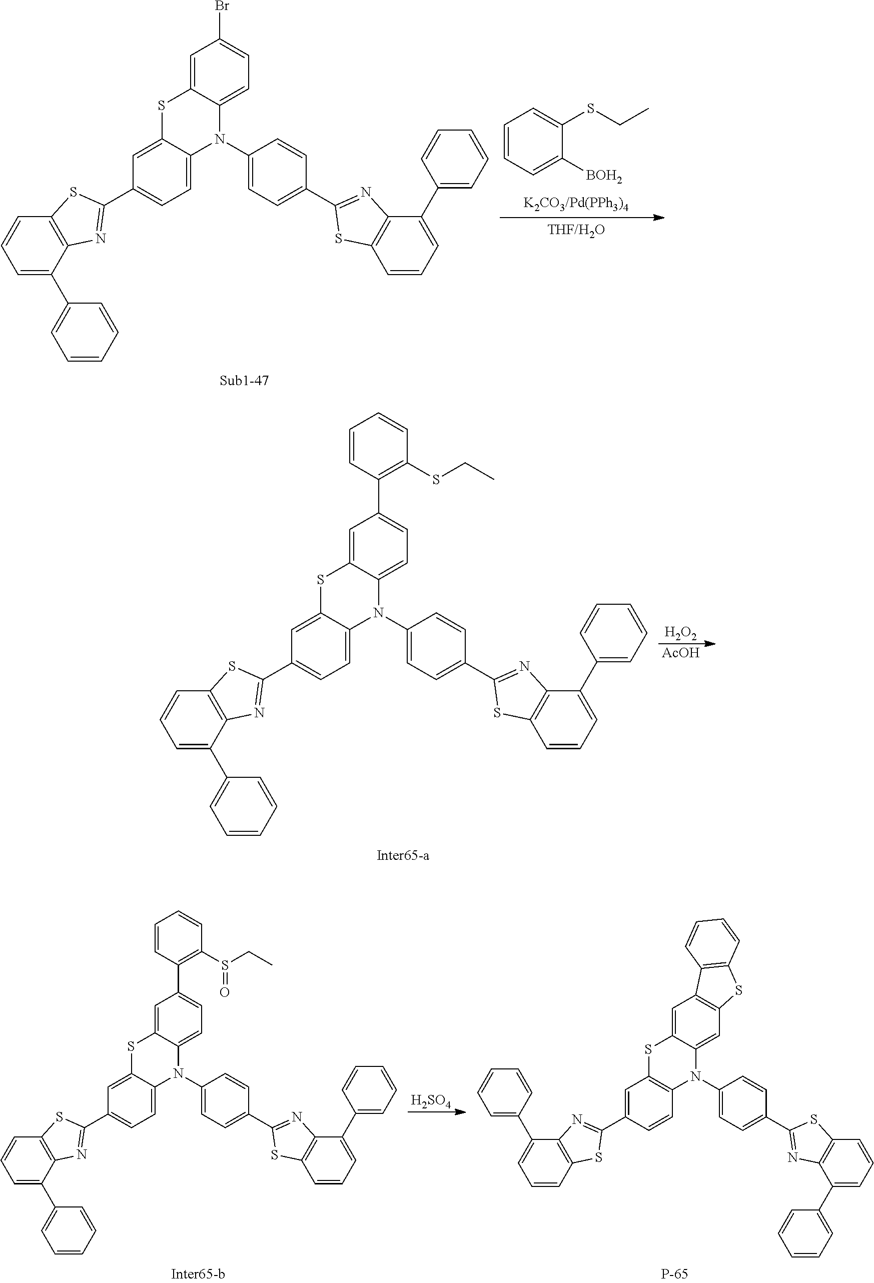

### (1) Synthesis of Inter 65-a

After dissolving Sub1-47 (4.0 g, 5.2 mmol) obtained in the above synthesis in THF (26 mL) in a round-bottom flask, 2-Methylsulfanylphenylboronic acid (0.9 g, 5.2 mmol), K₂CO₃ (2.1 g, 15.5 mmol), Pd(PPh₃)₄ (0.36 g, 0.31 mmol), water (13 mL) were added and the experiment was performed in the same manner as Inter1-a above to obtain 3.4 g of a product. (yield 78%)

### (2) Synthesis of Inter65-b

Acetic acid (16 mL) and 35% hydrogen peroxide (H₂O₂) (1.15 mL) were added to Inter65-a (3.4 g, 4.0 mmol) obtained in the above synthesis, and the experiment was performed in the same manner as for Inter1-b to obtain 3.1 g of the product. (yield 92%)

### (3) Synthesis of P-65

Sulfuric acid (H₂SO₄) (9.4 mL) was added to Inter65-b (3.1 g, 3.7 mmol) obtained in the above synthesis, and an experiment was performed in the same manner as in P-1 to obtain 2.8 g of a product. (yield 93%)

### 5. Synthesis Example of P-73

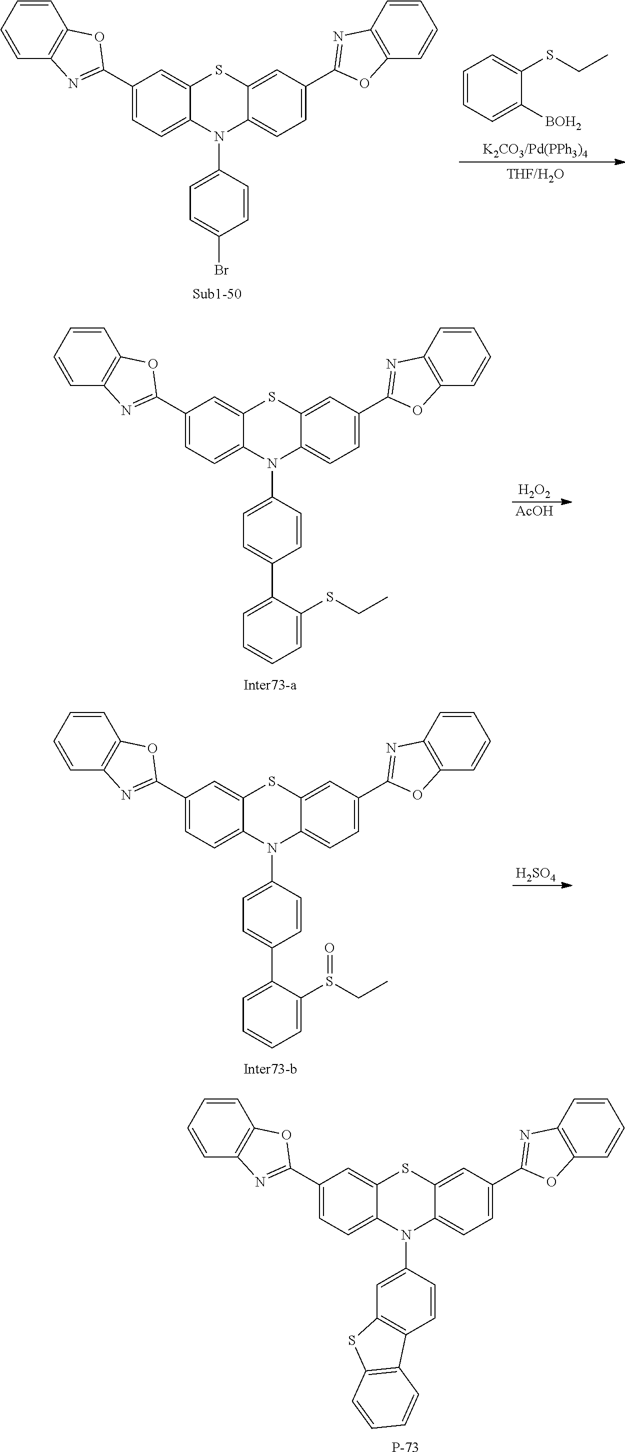

### (1) Synthesis of Inter73-a

After dissolving Sub1-50 (4.0 g, 6.8 mmol) obtained in the above synthesis in THF (34 mL) in a round-bottom flask, 2-Methylsulfanylphenylboronic acid (1.1 g, 6.8 mmol), K₂CO₃ (2.8 g, 20.4 mmol), Pd(PPh₃)₄ (0.47 g, 0.41 mmol), and water (17 mL) were added, and the same test as for Inter1-a was performed to obtain 3.7 g of a product. (yield 85%)

### (2) Synthesis of Inter73-b

Acetic acid (23 mL) and 35% hydrogen peroxide (H₂O₂) (1.65 mL) were added to Inter73-a (3.7 g, 5.8 mmol) obtained in the above synthesis, and the experiment was performed in the same manner as for Inter1-b to obtain 3.4 g of the product. (yield 89%)

### (3) Synthesis of P-73

Sulfuric acid (H2SO4) (10.2 mL) was added to Inter73-b (3.4 g, 5.1 mmol) obtained in the above synthesis, and an experiment was performed in the same manner as in P-1 to obtain 2.9 g of a product. (Yield 91%)

Meanwhile, FD-MS values of compounds P-1 to P-84 of the present invention prepared according to the above synthesis examples are shown in Table 2 below.

TABLE 2

| compound | FD-MS | compound | FD-MS |
|---|---|---|---|
| P-1 | m/z = 675.02(C₃₈H₁₇N₃O₂S₄ = 675.81) | P-2 | m/z = 659.04(C₃₈H₁₇N₃O₃S₃ = 659.75) |
| P-3 | m/z = 767.03(C₄₄H₂₁N₃OS₅ = 767.97) | P-4 | m/z = 687.14(C₄₄H₂₁N₃O₆ = 687.67) |
| P-5 | m/z = 699.13(C₄₂H₂₅N₃O₄S₂ = 699.8) | P-6 | m/z = 659.04(C₃₈H₁₇N₃O₃S₃ = 659.75) |
| P-7 | m/z = 699.05(C₃₈H₉D₈N₃OS₅ = 699.92) | P-8 | m/z = 704.12(C₄₃H₂₀N₄O₅S = 704.72) |
| P-9 | m/z = 756.99(C₄₂H₁₉N₃S₆ = 758) | P-10 | m/z = 756.99(C₄₂H₁₉N₃S₆ = 758) |
| P-11 | m/z = 661.13(C₄₂H₁₉N₃O₆ = 661.63) | P-12 | m/z = 711.14(C₄₆H₂₁N₃O₆ = 711.69) |
| P-13 | m/z = 613.11(C₃₈H₁₉N₃O₄S = 613.65) | P-14 | m/z = 631.1(C₃₈H₁₈FN₃O₄S = 631.64) |
| P-15 | m/z = 813.1(C₅₀H₂₇N₃OS₄ = 814.03) | P-16 | m/z = 645.06(C₃₈H₁₉N₃O₂S₃ = 645.77) |
| P-17 | m/z = 613.11(C₃₈H₁₉N₃O₄S = 613.65) | P-18 | m/z = 689.14(C₄₄H₂₃N₃O₄S = 689.75) |
| P-19 | m/z = 745.1(C₄₆H₂₃N₃O₂S₃ = 745.89) | P-20 | m/z = 613.11(C₃₈H₁₉N₃O₄S = 613.65) |
| P-21 | m/z = 663.13(C₄₂H₂₁N₃O₄S = 663.71) | P-22 | m/z = 679.1(C₄₂H₂₁N₃O₃S₂ = 679.77) |
| P-23 | m/z = 729.17(C₄₂H₂₇N₃O₄S = 729.81) | P-24 | m/z = 711.06(C₄₂H₂₁N₃OS₄ = 711.89) |
| P-25 | m/z = 613.11(C₃₈H₁₉N₃O₄S = 613.65) | P-26 | m/z = 689.14(C₄₄H₂₃N₃O₄ = 689.75) |
| P-27 | m/z = 679.08(C₄₀H₁₇N₅O₃S₂ = 679.73) | P-28 | m/z = 677.02(C₃₈H₁₉N₃S₅ = 677.89) |
| P-29 | m/z = 721.1(C₄₄H₂₃N₃O₂S₃ = 721.87) | P-30 | m/z = 622.13(C₃₉H₁₈N₄O₅ = 622.6) |
| P-31 | m/z = 797.18(C₅₁H₃₁N₃O₃S₂ = 797.95) | P-32 | m/z = 690.14(C₄₃H₂₂N₄O₄ = 690.73) |
| P-33 | m/z = 663.13(C₄₂H₂₁N₃O₄S = 663.71) | P-34 | m/z = 729.12(C₄₆H₂₃N₃O₃S₂ = 729.83) |
| P-35 | m/z = 829.15(C₅₄H₂₇N₃O₃S₂ = 829.95) | P-36 | m/z = 679.16(C₄₃H₂₅N₃O₄ = 679.75) |
| P-37 | m/z = 645.06(C₃₈H₁₉N₃O₂S₃ = 645.77) | P-38 | m/z = 597.13(C₃₈H₁₉N₃O₅ = 597.59) |
| P-39 | m/z = 677.07(C₃₉H₂₃N₃OS₄ = 677.87) | P-40 | m/z = 653.11(C₃₈H₁₁D₈N₃O₂S₃ = 653.82) |
| P-41 | m/z = 705.12(C₄₄H₂₃N₃O₃S₂ = 705.81) | P-42 | m/z = 767.16(C₄₈H₂₅N₅O₄S = 767.82) |
| P-43 | m/z = 886.15(C₅₆H₃₀N₄O₂S₃ = 887.06) | P-44 | m/z = 805.15(C₅₂H₂₂N₃O₃S₂ = 805.93) |
| P-45 | m/z = 679.1(C₄₂H₂₁N₃O₃S₂ = 679.77) | P-46 | m/z = 679.1(C₄₂H₂₁N₃O₃S₂ = 679.77) |
| P-47 | m/z = 679.1(C₄₂H₂₁N₃O₃S₂ = 679.77) | P-48 | m/z = 729.12(C₄₆H₂₃N₃O₃S₂ = 729.83) |
| P-49 | m/z = 615.11(C₃₈H₂₁N₃O₂S₂ = 615.73) | P-50 | m/z = 599.13(C₃₈H₂₁N₃O₃S = 599.66) |
| P-51 | m/z = 647.12(C₃₉H₂₅N₃OS₃ = 647.83) | P-52 | m/z = 615.11(C₃₈H₂₁N₃O₂S₂ = 615.73) |
| P-53 | m/z = 675.16(C₄₄H₂₅N₃O₃S = 675.76) | P-54 | m/z = 707.13(C₄₄H₂₅N₃O₃S₂ = 707.82) |
| P-55 | m/z = 791.22(C₅₃H₃₃N₃O₃S = 791.93) | P-56 | m/z = 619.13(C₃₈H₁₇D₄N₃O₂S₂ = 619.75) |
| P-57 | m/z = 649.15(C₄₂H₂₃N₃O₃S = 649.72) | P-58 | m/z = 699.22(C₄₂H₂₉N₃O₄ = 699.77) |
| P-59 | m/z = 697.08(C₄₂H₂₃N₃S₄ = 697.91) | P-60 | m/z = 697.08(C₄₂H₂₃N₃S₄ = 697.91) |
| P-61 | m/z = 615.11(C₃₈H₂₁N₃O₂S₂ = 615.73) | P-62 | m/z = 631.08(C₃₈H₂₁N₃OS₃ = 631.79) |
| P-63 | m/z = 615.11(C₃₈H₂₁N₃O₂S₂ = 615.73) | P-64 | m/z = 599.18(C₃₉H₂₅N₃O₄ = 599.65) |
| P-65 | m/z = 799.12(C₅₀H₂₉N₃S₄ = 800.04) | P-66 | m/z = 691.19(C₄₅H₂₉N₃O₃S = 691.81) |
| P-67 | m/z = 781.15(C₅₀H₂₇N₃O₃S₂ = 781.9) | P-68 | m/z = 683.18(C₄₆H₂₅N₃O₄ = 683.72) |
| P-69 | m/z = 649.15(C₄₂H₂₃N₃O₃S = 649.72) | P-70 | m/z = 697.08(C₄₂H₂₃N₃S₄ = 697.91) |
| P-71 | m/z = 697.08(C₄₂H₂₃N₃S₄ = 697.91) | P-72 | m/z = 649.2(C₄₃H₂₇N₃O₄ = 649.71) |
| P-73 | m/z = 615.11(C₃₈H₂₁N₃O₂S₂ = 615.73) | P-74 | m/z = 615.11(C₃₈H₂₁N₃O₂S₂ = 615.73) |
| P-75 | m/z = 631.08(C₃₈H₂₁N₃OS₃ = 631.79) | P-76 | m/z = 599.18(C₃₉H₂₅N₃O₄ = 599.65) |
| P-77 | m/z = 846.19(C₅₃H₃₀N₆O₂S₂ = 846.98) | P-78 | m/z = 675.16(C₄₄H₂₅N₃O₃S = 675.76) |
| P-79 | m/z = 599.13(C₃₈H₂₁N₃O₃S = 599.66) | P-80 | m/z = 587.18(C₃₈H₁₇D₄N₃O₄ = 587.63) |
| P-81 | m/z = 681.1(C₄₂H₂₃N₃OS₃ = 681.85) | P-82 | m/z = 649.15(C₄₂H₂₃N₃O₃S = 649.72) |
| P-83 | m/z = 649.15(C₄₂H₂₃N₃O₃S = 649.72) | P-84 | m/z = 683.18(C₄₆H₂₅N₃O₄ = 683.72) |

### Manufacturing Evaluation of Organic Electronic Element

### [Example 1] Green Organic Light Emitting Device (Light Efficiency Enhancing Layer)

An organic electroluminescent device was manufactured according to a conventional method using the compound of the present invention as a light efficiency enhancing layer material. First, on the ITO layer (anode) formed on the glass substrate, N1-(naphthalen-2-yl)-N4,N4-bis(4-(naphthalen-2-yl(phenyl)amino)phenyl)-N1-phenylbenzene-1,4-diamine (abbreviated as 2-TNATA) film as a hole injection layer was vacuum-deposited to form a thickness of 60 nm. Then, 4,4-bis[N-(1-naphthyl)-N-phenylamino]biphenyl (abbreviated as NPD) as a hole transport compound was vacuum-deposited on the film to a thickness of 60 nm to form a hole transport layer. Then, an emitting layer with a thickness of 30 nm was deposited on the emitting-auxiliary layer by doping CBP[4,4′-N,N′-dicarbazole-biphenyl] as a host and Ir(ppy)₃ [tris(2-phenylpyridine)-iridium] as a dopant at a weight of 95:5 on the hole transport layer. Vacuum-depositing (1,1′-bisphenyl)-4-oleato)bis(2-methyl-8-quinolineoleato)aluminum (hereinafter abbreviated as BAlq) to a thickness of 10 nm as a hole blocking layer, and tris(8-quinolinol)aluminum (hereinafter, abbreviated as Alq3) was deposited to a thickness of 40 nm to form an electron transport layer. LiF, which is an alkali metal halide, is deposited on the electron transport layer to a thickness of 0.2 nm to form an electron injection layer, and then Al was deposited to a thickness of 150 nm and used as a cathode, and an organic electroluminescent device was prepared by forming a light efficiency enhancing layer on the compound of the present invention represented by Formula 1 to a thickness of 60 nm.

### [Example 2] to [Example 13] Green Organic Light Emitting Device

An organic electroluminescent device was manufactured in the same manner as in Example 1, except that the compound of the present invention described in Table 3 was used instead of the compound P-1 of the present invention as the light efficiency enhancing layer material.

### Comparative Example 1

An organic electroluminescent device was manufactured in the same manner as in Example 1, except that the comparative compound A was used instead of the compound P-1 of the present invention as a material for the light efficiency enhancing layer.

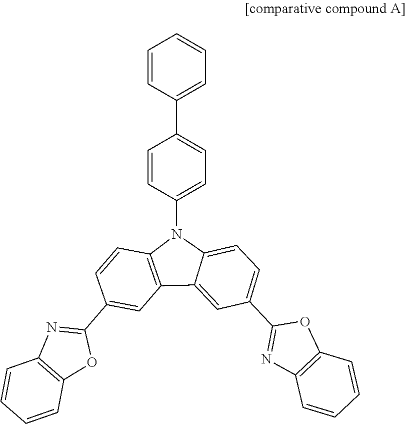 [SMILES: c1ccc(-c2ccc(-n3c4ccc(-c5nc6ccccc6o5)cc4c4cc(-c5nc6ccccc6o5)ccc43)cc2)cc1]

By applying a forward bias DC voltage to the organic electroluminescent devices prepared in Examples and Comparative Examples prepared in this way, Electroluminescence (EL) characteristics were measured with PR-650 from photo research, and as a result of the measurement, the T95 lifetime was measured using a lifetime measuring device manufactured by McScience at 5000 cd/m² standard luminance. Table 3 below shows the device fabrication and evaluation results.

TABLE 3

| | | | Current | | | | | |
|---|---|---|---|---|---|---|---|---|
| | | | Density | Brightness | Efficiency | | CIE | |
| | compound | Voltage | (mA/cm²) | (cd/m²) | (cd/A) | T(95) | x | Y |
| comparative | — | 6.4 | 21.5 | 5000 | 23.3 | 69.6 | 0.34 | 0.61 |
| example(1) | | | — | | | | | |
| comparative | comparative | 6.3 | 15.3 | 5000 | 32.7 | 70.2 | 0.34 | 0.64 |
| example(2) | compound A | | | | | | | |
| example(1) | compound(P-1) | 6.2 | 10.6 | 5000 | 47.4 | 71.0 | 0.33 | 0.64 |
| example(2) | compound(P-4) | 6.3 | 11.6 | 5000 | 43.0 | 70.9 | 0.33 | 0.64 |
| example(3) | compound(P-11) | 6.3 | 11.5 | 5000 | 43.6 | 70.5 | 0.33 | 0.64 |
| example(4) | compound(P-13) | 6.3 | 11.3 | 5000 | 44.3 | 71.0 | 0.34 | 0.65 |
| example(5) | compound(P-17) | 6.2 | 11.5 | 5000 | 43.4 | 70.4 | 0.34 | 0.65 |
| example(6) | compound(P-21) | 6.2 | 11.1 | 5000 | 44.9 | 70.6 | 0.34 | 0.65 |
| example(7) | compound(P-37) | 6.3 | 11.1 | 5000 | 45.1 | 71.2 | 0.33 | 0.64 |
| example(8) | compound(P-49) | 6.3 | 11.4 | 5000 | 43.8 | 70.5 | 0.33 | 0.65 |
| example(9) | compound(P-61) | 6.3 | 11.4 | 5000 | 43.7 | 71.2 | 0.33 | 0.64 |
| example(10) | compound(P-62) | 6.2 | 11.5 | 5000 | 43.3 | 70.9 | 0.34 | 0.65 |
| example(11) | compound(P-65) | 6.3 | 11.1 | 5000 | 45.0 | 70.7 | 0.34 | 0.64 |
| example(12) | compound(P-73) | 6.2 | 11.2 | 5000 | 44.4 | 70.8 | 0.34 | 0.65 |
| example(13) | compound(P-79) | 6.2 | 11.8 | 5000 | 42.3 | 71.0 | 0.33 | 0.64 |

As can be seen from the results of Table 3, when the organic electroluminescent device including the compound of the present invention is configured as the light efficiency enhancing layer, high color purity and luminous efficiency can be significantly improved.

Furthermore, when comparing the results of the device with and without the light efficiency enhancing layer, it can be seen that the color purity and efficiency increase with the light efficiency enhancing layer, and that efficiency is significantly improved when the compound of the present invention is used compared to when the light efficiency enhancing layer is Comparative Compound A.

When the light efficiency enhancing layer is used, SPPs (Surface plasmon polaritons) are generated at the interface between the Al electrode and the high refractive organic material, and among them, TE (transverse electric) polarized light is annihilated from the light efficiency enhancing layer in the vertical direction by evanescent waves, and TM (transverse magnetic) polarized light moving along the cathode and the light efficiency enhancing layer is amplified by surface plasma resonance, enabling high efficiency and effective color purity control.

As described above, in the visible light wavelength band (range of 430 nm to 780 nm), the compound of the present invention has a high refractive index of 1.8 to 2.1, accordingly, the efficiency of light generated in the organic layer being extracted to the outside of the organic light emitting device is increased by the principle of constructive interference, thereby greatly contributing to the improvement of the light efficiency of the organic light emitting device.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

BRIEF DESCRIPTION OF THE DRAWINGS

| | |
|---|---|
| 1001 200, 300: organic electronic | 110: the first electrode |
| element | |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting-auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

1. A compound represented by Formula 1: 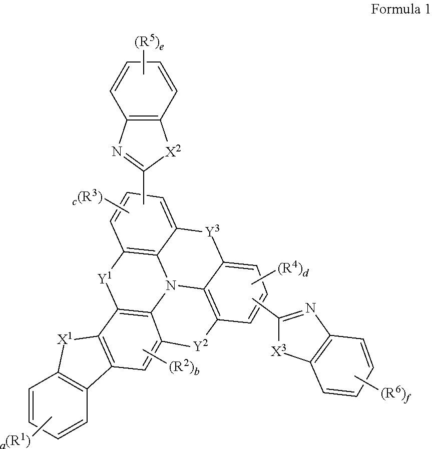
wherein:
1) X¹, X² and X³ are each independently O or S,
2) Y¹, Y² and Y³ are each independently O, S or absent,
3) R¹, R², R³, R⁴, R⁵ and R⁶ are each the same or different, and each independently selected from a group consisting of hydrogen; deuterium; halogen; cyano group; nitro group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; a C₆-C₆₀ aryloxy group; and -L′-N(R^{a})(R^{b}); or a plurality of adjacent R¹s, or a plurality of R²s, or a plurality of R³s, a plurality of adjacent R⁴s, or a plurality of R⁵s, or a plurality of R⁶s may be bonded to each other to form a ring,
4) wherein L′ is each independently selected from the group consisting of a single bond; a C₆-C₆₀ arylene group; a fluorenylene group; a C₂-C₆₀ heterocyclic group including at least one heteroatom of O, N, S, Si or P; and a C₃-C₆₀ aliphatic ring; and R^{a} and R^{b} are each independently a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring,
5) a, c, d, e and f are each independently an integer from 0 to 4, and b is an integer from 0 to 3,
6) wherein the aryl group, arylene group, heterocyclic group, fluorenyl group, fluorenylene group, fused ring group, alkyl group, alkenyl group, alkoxy group and aryloxy group in any of R¹ to R⁶, L′, R^{a} and R^{b} may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; C₈-C₂₀ arylalkenyl group; and -L′-NR^{a}R^{b}; and the substituents may be bonded to each other to form a C₃-C₆₀ aliphatic ring or a C₆-C₆₀ aromatic ring or a C₂-C₆₀ heterocyclic group or a fused ring formed by the combination thereof.

2. The compound of claim 1, wherein the compound represented by Formula 1 is represented by any one of Formulas 2 to 4: 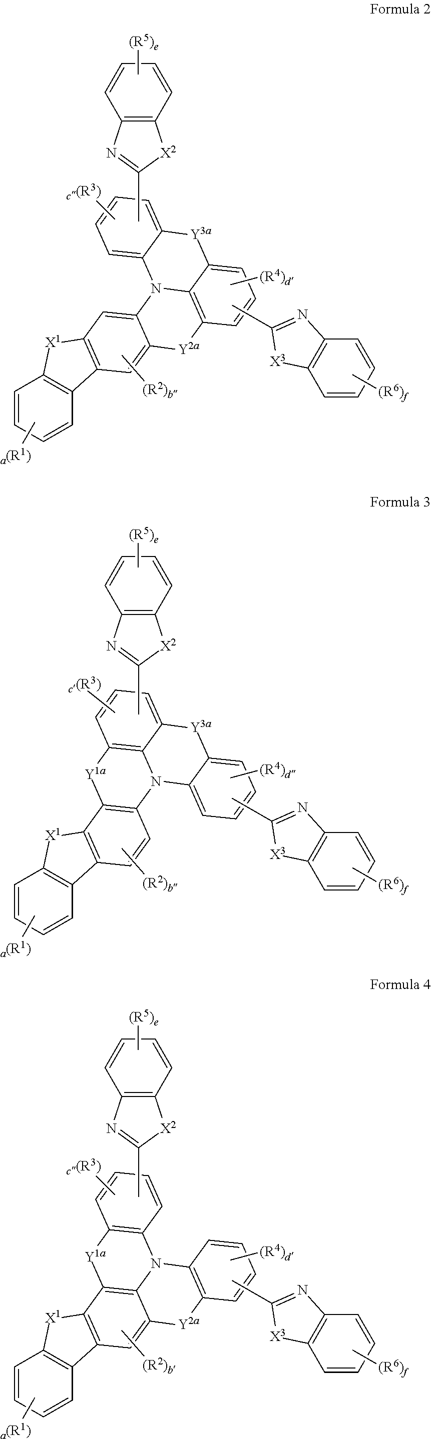
wherein:
1) X¹, X², X³, R¹, R², R³, R⁴, R⁵, R⁶, a, e and f are the same as defined for Formula 1,
2) Y^{1a}, Y^{2a} and Y^{3a} are each independently O or S,
3) b′ is 0 or 1, and b″, c′ and d′ are independently of each other an integer of 0 to 2, and
c″ and d″ are independently of each other an integer from 0 to 3.

3. The compound of claim 1, wherein the compound represented by Formula 1 is represented by any one of Formulas 5 to 7: 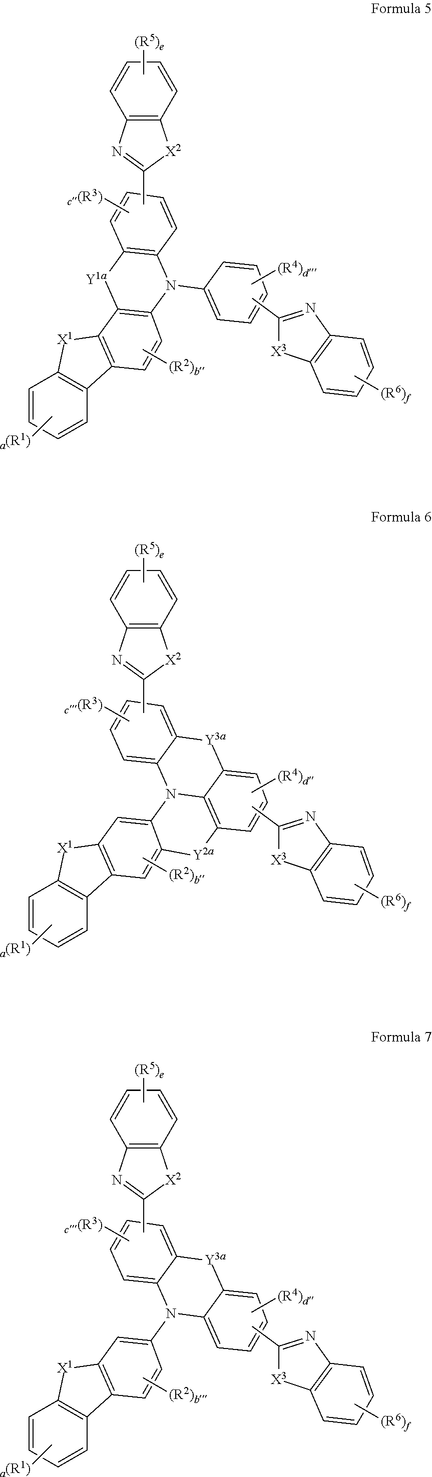
wherein:
1) X¹, X², X³, R¹, R², R³, R⁴, R⁵, R⁶, a, e and f are the same as defined for Formula 1,
2) Y^{1a}, Y^{2a}, Y^{3a} are each independently O or S, b″ is an integer of 0 to 2, and c″ and d″ are each independently an integer of 0 to 3,
3) b′″ is an integer from 0 to 3, and c′″ and d′″ are independently n integer from 0 to 4.

4. The compound of claim 1, wherein the compound represented by Formula 1 is any one of the following compounds:

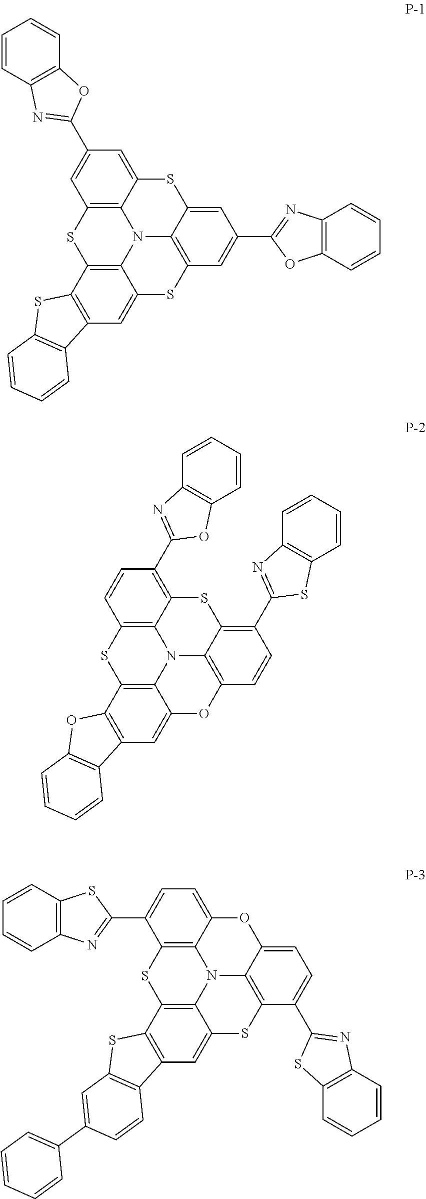 [SMILES: c1ccc2oc(-c3cc4c5c(c3)Sc3cc6c(sc7ccccc76)c6c3N5c3c(cc(-c5nc7ccccc7o5)cc3S6)S4)nc2c1 ; c1ccc2oc(-c3ccc4c5c3Sc3c(-c6nc7ccccc7s6)ccc6c3N5c3c(cc5c(oc7ccccc75)c3S4)O6)nc2c1 ; c1ccc(-c2ccc3c(c2)sc2c4c5c(cc23)Sc2c(-c3nc6ccccc6s3)ccc3c2N5c2c(ccc(-c5nc6ccccc6s5)c2S4)O3)cc1]

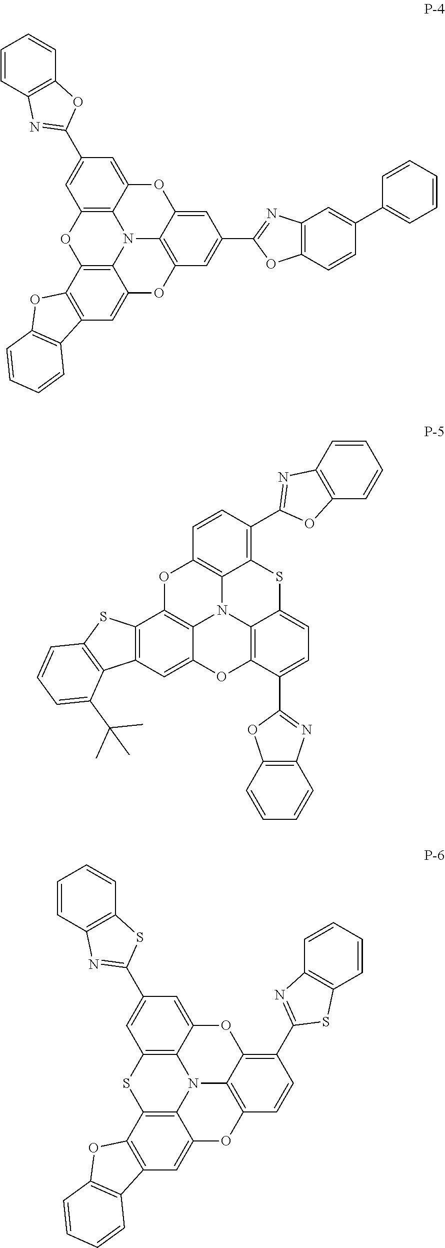 [SMILES: c1ccc(-c2ccc3oc(-c4cc5c6c(c4)Oc4cc7c(oc8ccccc87)c7c4N6c4c(cc(-c6nc8ccccc8o6)cc4O7)O5)nc3c2)cc1 ; CC(C)(C)c1cccc2sc3c4c5c(cc3c12)Oc1c(-c2nc3ccccc3o2)ccc2c1N5c1c(ccc(-c3nc5ccccc5o3)c1S2)O4 ; c1ccc2sc(-c3cc4c5c(c3)Sc3c6c(cc7c3oc3ccccc37)Oc3ccc(-c7nc8ccccc8s7)c(c3N56)O4)nc2c1]

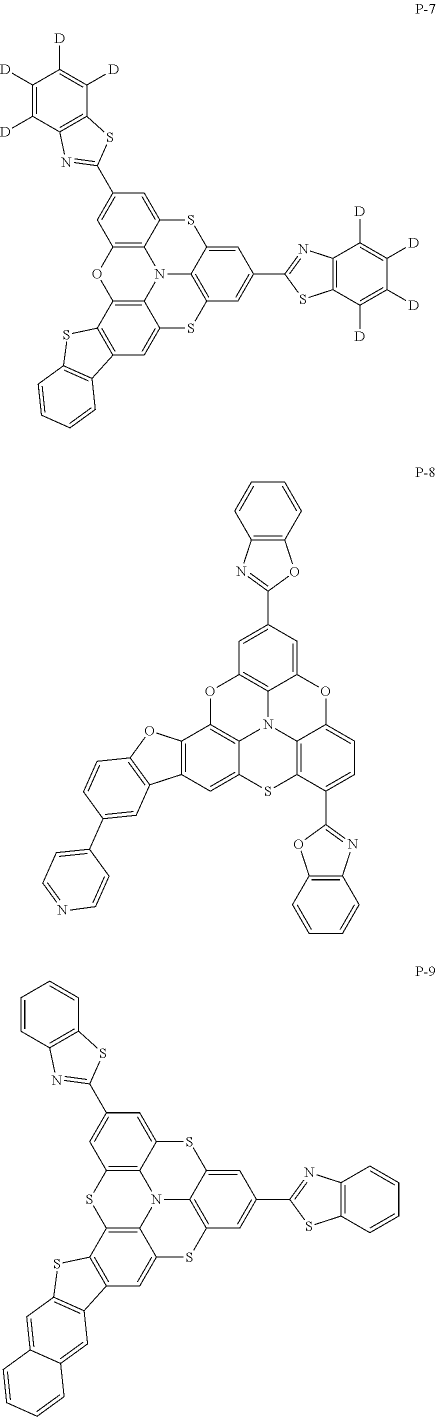 [SMILES: [2H]c1c([2H])c([2H])c2sc(-c3cc4c5c(c3)Sc3cc(-c6nc7c([2H])c([2H])c([2H])c([2H])c7s6)cc6c3N5c3c(cc5c(sc7ccccc75)c3O4)S6)nc2c1[2H] ; c1ccc2oc(-c3cc4c5c(c3)Oc3c6c(cc7c3oc3ccc(-c8ccncc8)cc37)Sc3c(-c7nc8ccccc8o7)ccc(c3N56)O4)nc2c1 ; c1ccc2cc3c(cc2c1)sc1c2c4c(cc13)Sc1cc(-c3nc5ccccc5s3)cc3c1N4c1c(cc(-c4nc5ccccc5s4)cc1S2)S3]

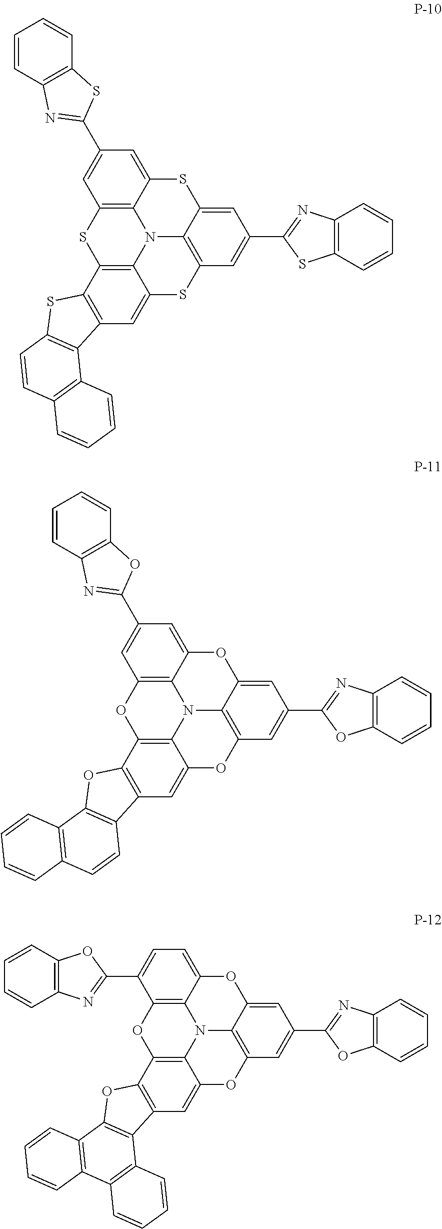

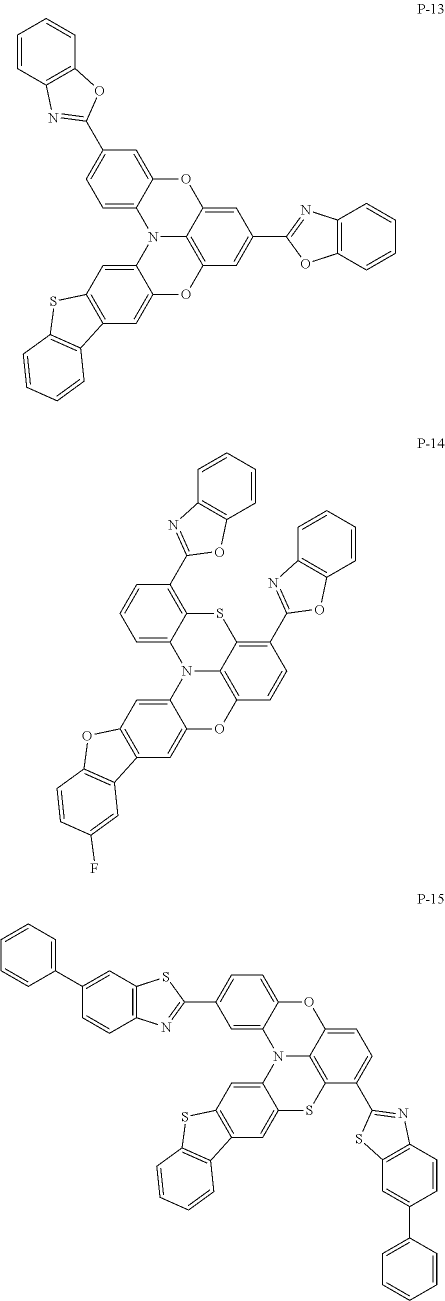 [SMILES: c1ccc2oc(-c3ccc4c(c3)Oc3cc(-c5nc6ccccc6o5)cc5c3N4c3cc4sc6ccccc6c4cc3O5)nc2c1 ; Fc1ccc2oc3cc4c(cc3c2c1)Oc1ccc(-c2nc3ccccc3o2)c2c1N4c1cccc(-c3nc4ccccc4o3)c1S2 ; c1ccc(-c2ccc3nc(-c4ccc5c(c4)N4c6cc7sc8ccccc8c7cc6Sc6c(-c7nc8ccc(-c9ccccc9)cc8s7)ccc(c64)O5)sc3c2)cc1]

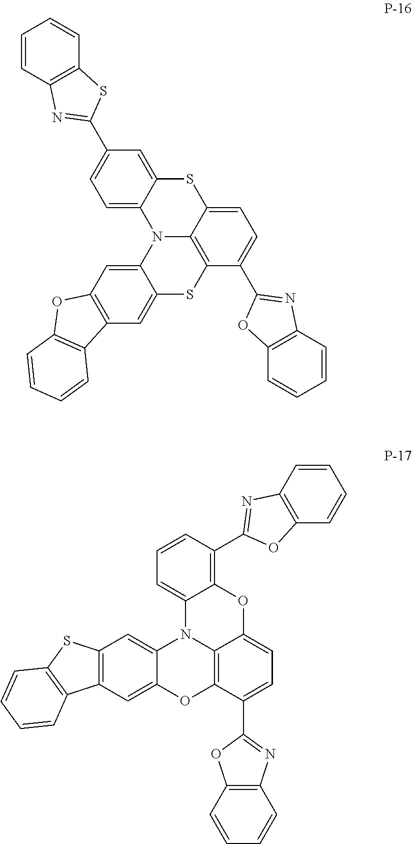 [SMILES: c1ccc2oc(-c3ccc4c5c3Sc3cc6c(cc3N5c3ccc(-c5nc7ccccc7s5)cc3S4)oc3ccccc36)nc2c1 ; c1cc(-c2nc3ccccc3o2)c2c(c1)N1c3cc4sc5ccccc5c4cc3Oc3c(-c4nc5ccccc5o4)ccc(c31)O2]

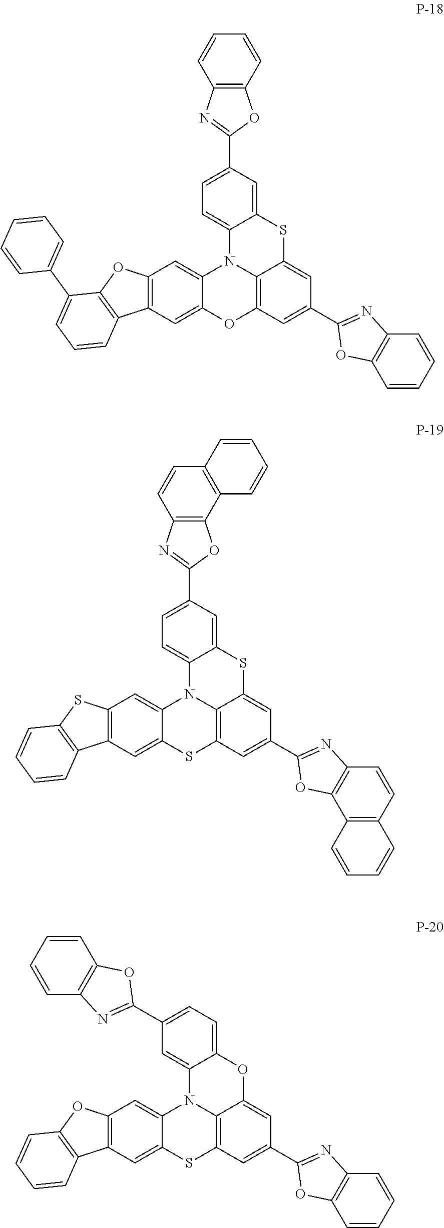 [SMILES: c1ccc(-c2cccc3c2oc2cc4c(cc23)Oc2cc(-c3nc5ccccc5o3)cc3c2N4c2ccc(-c4nc5ccccc5o4)cc2S3)cc1 ; c1ccc2c(c1)ccc1nc(-c3ccc4c(c3)Sc3cc(-c5nc6ccc7ccccc7c6o5)cc5c3N4c3cc4sc6ccccc6c4cc3S5)oc12 ; c1ccc2oc(-c3cc4c5c(c3)Sc3cc6c(cc3N5c3cc(-c5nc7ccccc7o5)ccc3O4)oc3ccccc36)nc2c1]

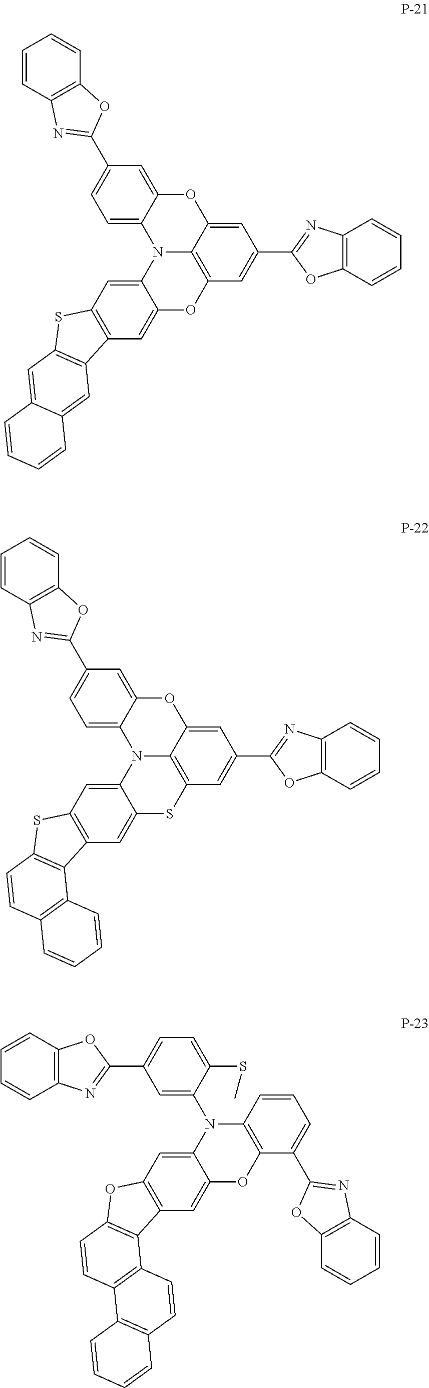 [SMILES: c1ccc2cc3c(cc2c1)sc1cc2c(cc13)Oc1cc(-c3nc4ccccc4o3)cc3c1N2c1ccc(-c2nc4ccccc4o2)cc1O3 ; c1ccc2c(c1)ccc1sc3cc4c(cc3c12)Sc1cc(-c2nc3ccccc3o2)cc2c1N4c1ccc(-c3nc4ccccc4o3)cc1O2 ; CSc1ccc(-c2nc3ccccc3o2)cc1N1c2cc3oc4ccc5c6ccccc6ccc5c4c3cc2Oc2c(-c3nc4ccccc4o3)cccc21]

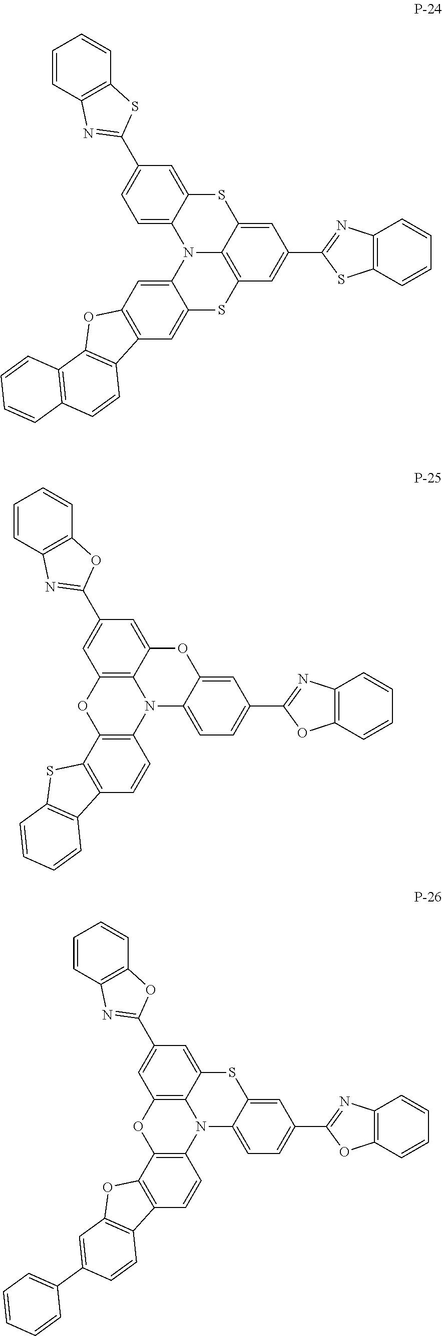 [SMILES: c1ccc2c(c1)ccc1c3cc4c(cc3oc21)N1c2ccc(-c3nc5ccccc5s3)cc2Sc2cc(-c3nc5ccccc5s3)cc(c21)S4 ; c1ccc2oc(-c3ccc4c(c3)Oc3cc(-c5nc6ccccc6o5)cc5c3N4c3ccc4c(sc6ccccc64)c3O5)nc2c1 ; c1ccc(-c2ccc3c(c2)oc2c4c(ccc23)N2c3ccc(-c5nc6ccccc6o5)cc3Sc3cc(-c5nc6ccccc6o5)cc(c32)O4)cc1]

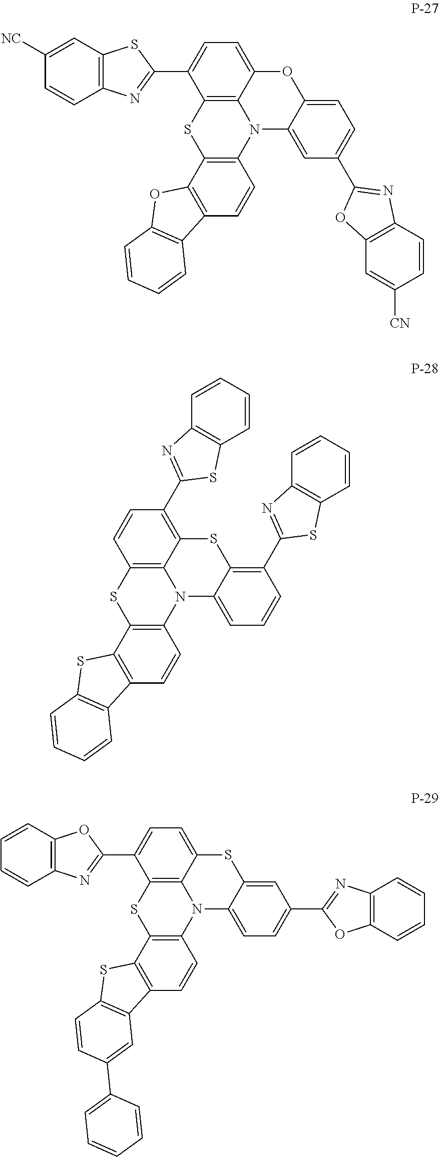 [SMILES: N#Cc1ccc2nc(-c3ccc4c(c3)N3c5ccc6c(oc7ccccc76)c5Sc5c(-c6nc7ccc(C#N)cc7s6)ccc(c53)O4)oc2c1 ; c1cc(-c2nc3ccccc3s2)c2c(c1)N1c3ccc4c(sc5ccccc54)c3Sc3ccc(-c4nc5ccccc5s4)c(c31)S2 ; c1ccc(-c2ccc3sc4c5c(ccc4c3c2)N2c3ccc(-c4nc6ccccc6o4)cc3Sc3ccc(-c4nc6ccccc6o4)c(c32)S5)cc1]

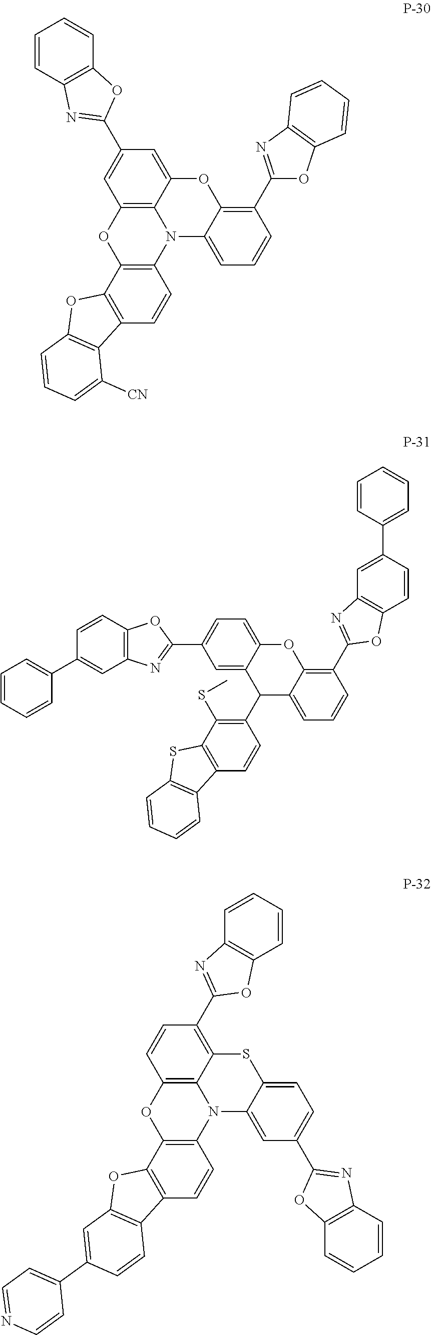 [SMILES: N#Cc1cccc2oc3c4c(ccc3c12)N1c2cccc(-c3nc5ccccc5o3)c2Oc2cc(-c3nc5ccccc5o3)cc(c21)O4 ; CSc1c(C2c3cc(-c4nc5cc(-c6ccccc6)ccc5o4)ccc3Oc3c(-c4nc5cc(-c6ccccc6)ccc5o4)cccc32)ccc2c1sc1ccccc12 ; c1ccc2oc(-c3ccc4c(c3)N3c5ccc6c(oc7cc(-c8ccncc8)ccc76)c5Oc5ccc(-c6nc7ccccc7o6)c(c53)S4)nc2c1]

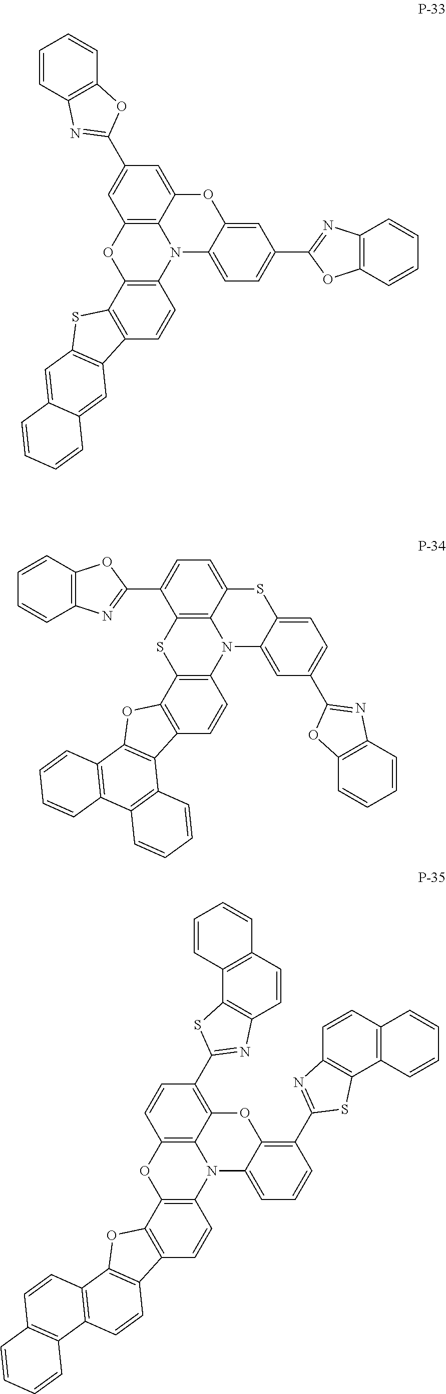 [SMILES: c1ccc2cc3c(cc2c1)sc1c2c(ccc13)N1c3ccc(-c4nc5ccccc5o4)cc3Oc3cc(-c4nc5ccccc5o4)cc(c31)O2 ; c1ccc2oc(-c3ccc4c(c3)N3c5ccc6c(oc7c8ccccc8c8ccccc8c67)c5Sc5c(-c6nc7ccccc7o6)ccc(c53)S4)nc2c1 ; c1cc(-c2nc3ccc4ccccc4c3s2)c2c(c1)N1c3ccc4c(oc5c4ccc4c6ccccc6ccc45)c3Oc3ccc(-c4nc5ccc6ccccc6c5s4)c(c31)O2]

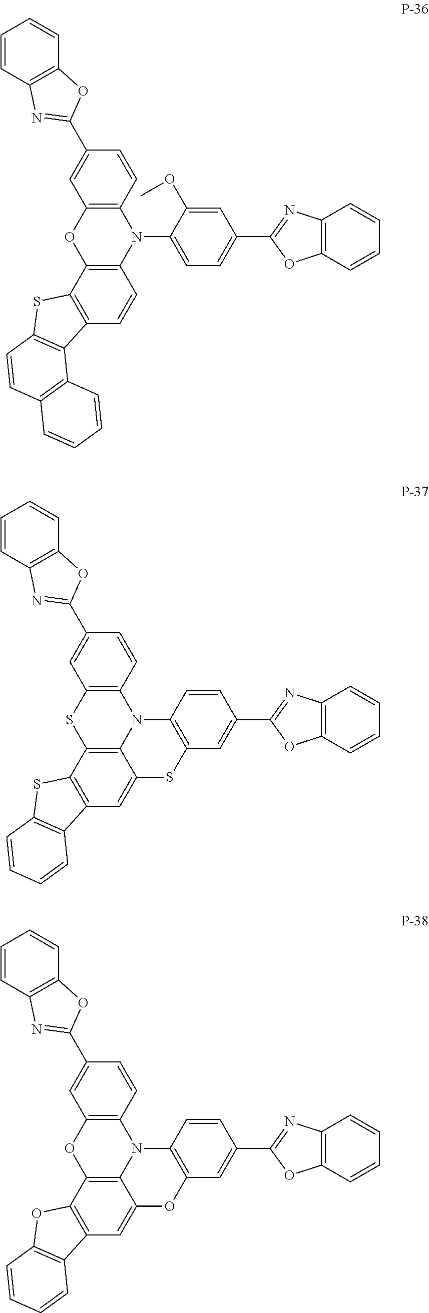 [SMILES: COc1cc(-c2nc3ccccc3o2)ccc1N1c2ccc(-c3nc4ccccc4o3)cc2Oc2c1ccc1c2sc2ccc3ccccc3c21 ; c1ccc2oc(-c3ccc4c(c3)Sc3cc5c(sc6ccccc65)c5c3N4c3ccc(-c4nc6ccccc6o4)cc3S5)nc2c1 ; c1ccc2oc(-c3ccc4c(c3)Oc3cc5c(oc6ccccc65)c5c3N4c3ccc(-c4nc6ccccc6o4)cc3O5)nc2c1]

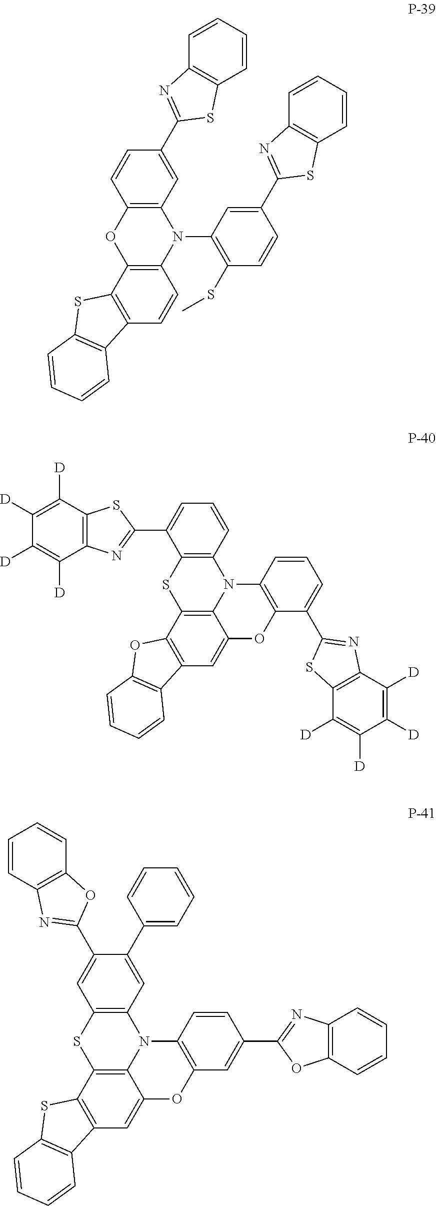 [SMILES: CSc1ccc(-c2nc3ccccc3s2)cc1N1c2cc(-c3nc4ccccc4s3)ccc2Oc2c1ccc1c2sc2ccccc21 ; [2H]c1c([2H])c([2H])c2sc(-c3cccc4c3Oc3cc5c(oc6ccccc65)c5c3N4c3cccc(-c4nc6c([2H])c([2H])c([2H])c([2H])c6s4)c3S5)nc2c1[2H] ; c1ccc(-c2cc3c(cc2-c2nc4ccccc4o2)Sc2c4c(cc5c2sc2ccccc25)Oc2cc(-c5nc6ccccc6o5)ccc2N34)cc1]

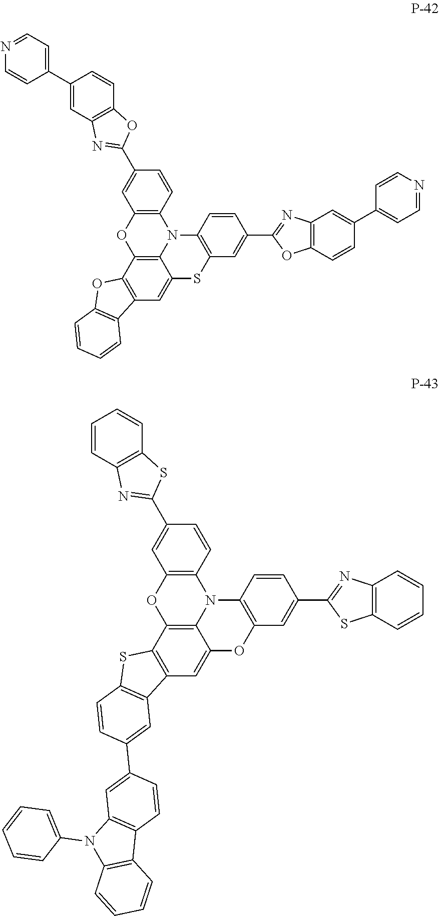 [SMILES: c1ccc2c(c1)oc1c3c4c(cc12)Sc1cc(-c2nc5cc(-c6ccncc6)ccc5o2)ccc1N4c1ccc(-c2nc4cc(-c5ccncc5)ccc4o2)cc1O3 ; c1ccc(-n2c3ccccc3c3ccc(-c4ccc5sc6c7c8c(cc6c5c4)Oc4cc(-c5nc6ccccc6s5)ccc4N8c4ccc(-c5nc6ccccc6s5)cc4O7)cc32)cc1]

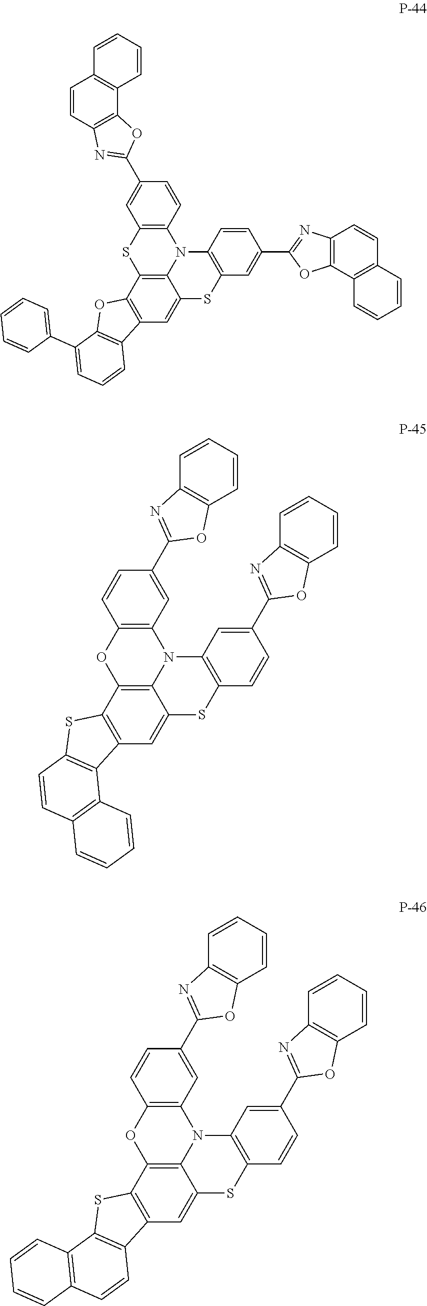 [SMILES: c1ccc(-c2cccc3c2oc2c4c5c(cc23)Sc2cc(-c3nc6ccc7ccccc7c6o3)ccc2N5c2ccc(-c3nc5ccc6ccccc6c5o3)cc2S4)cc1 ; c1ccc2c(c1)ccc1sc3c4c5c(cc3c12)Sc1ccc(-c2nc3ccccc3o2)cc1N5c1cc(-c2nc3ccccc3o2)ccc1O4 ; c1ccc2c(c1)ccc1c3cc4c5c(c3sc21)Oc1ccc(-c2nc3ccccc3o2)cc1N5c1cc(-c2nc3ccccc3o2)ccc1S4]

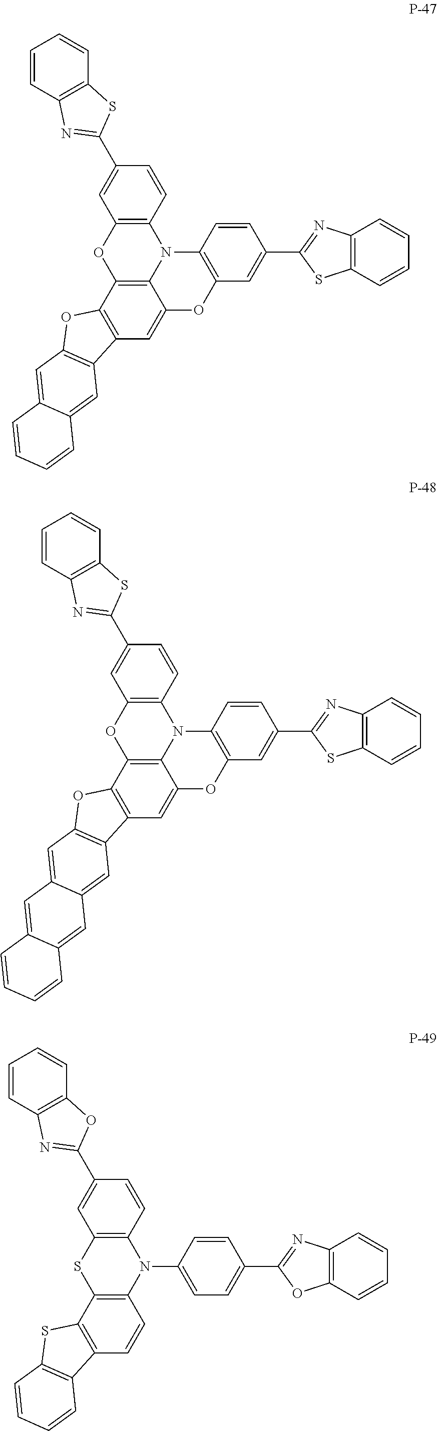 [SMILES: c1ccc2cc3c(cc2c1)oc1c2c4c(cc13)Oc1cc(-c3nc5ccccc5s3)ccc1N4c1ccc(-c3nc4ccccc4s3)cc1O2 ; c1ccc2cc3cc4c(cc3cc2c1)oc1c2c3c(cc14)Oc1cc(-c4nc5ccccc5s4)ccc1N3c1ccc(-c3nc4ccccc4s3)cc1O2 ; c1ccc2oc(-c3ccc(N4c5ccc(-c6nc7ccccc7o6)cc5Sc5c4ccc4c5sc5ccccc54)cc3)nc2c1]

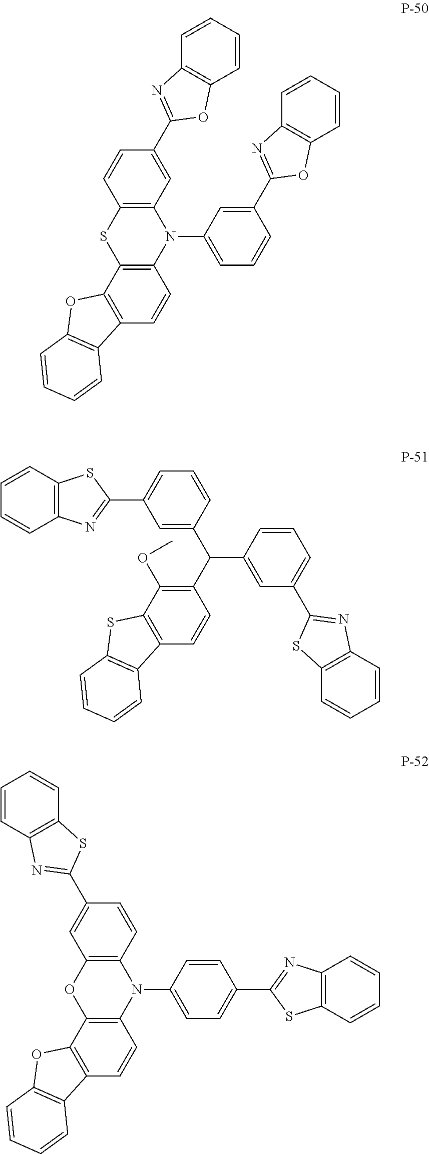 [SMILES: c1cc(-c2nc3ccccc3o2)cc(N2c3cc(-c4nc5ccccc5o4)ccc3Sc3c2ccc2c3oc3ccccc32)c1 ; COc1c(C(c2cccc(-c3nc4ccccc4s3)c2)c2cccc(-c3nc4ccccc4s3)c2)ccc2c1sc1ccccc12 ; c1ccc2sc(-c3ccc(N4c5ccc(-c6nc7ccccc7s6)cc5Oc5c4ccc4c5oc5ccccc54)cc3)nc2c1]

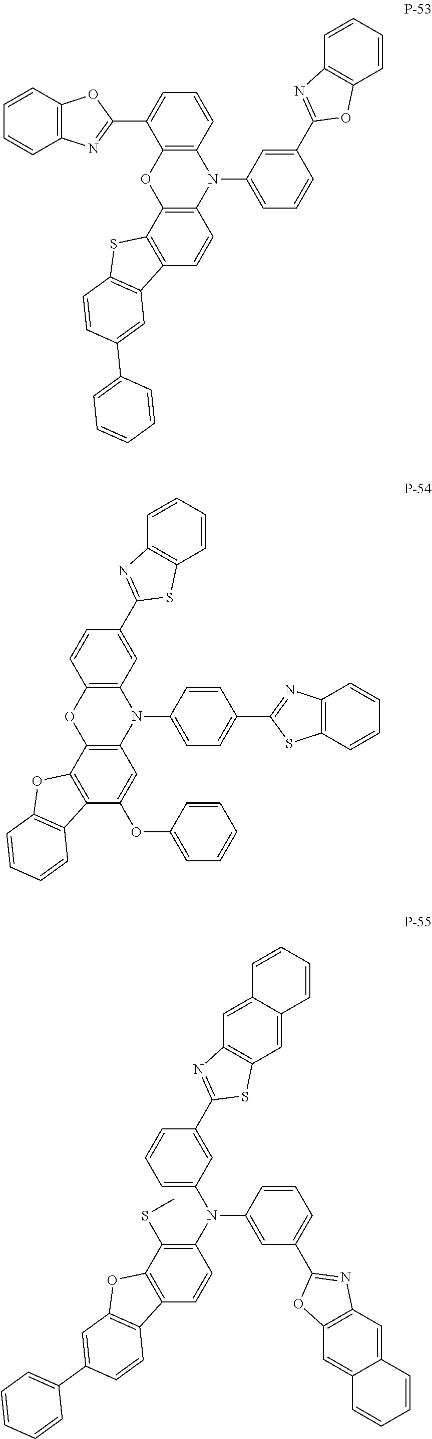 [SMILES: c1ccc(-c2ccc3sc4c5c(ccc4c3c2)N(c2cccc(-c3nc4ccccc4o3)c2)c2cccc(-c3nc4ccccc4o3)c2O5)cc1 ; c1ccc(Oc2cc3c(c4oc5ccccc5c24)Oc2ccc(-c4nc5ccccc5s4)cc2N3c2ccc(-c3nc4ccccc4s3)cc2)cc1 ; CSc1c(N(c2cccc(-c3nc4cc5ccccc5cc4o3)c2)c2cccc(-c3nc4cc5ccccc5cc4s3)c2)ccc2c1oc1cc(-c3ccccc3)ccc12]

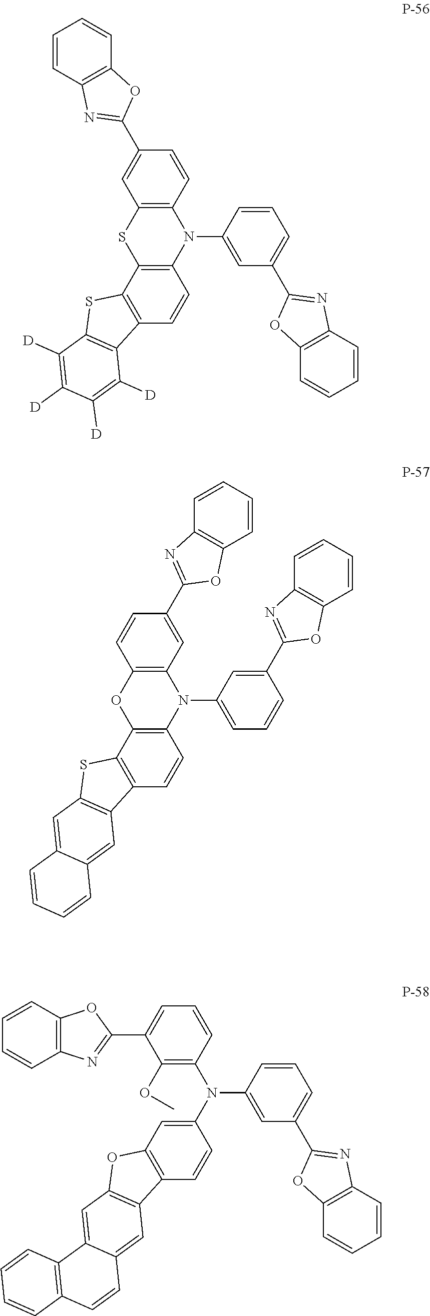 [SMILES: [2H]c1c([2H])c([2H])c2c(sc3c4c(ccc32)N(c2cccc(-c3nc5ccccc5o3)c2)c2ccc(-c3nc5ccccc5o3)cc2S4)c1[2H] ; c1cc(-c2nc3ccccc3o2)cc(N2c3cc(-c4nc5ccccc5o4)ccc3Oc3c2ccc2c3sc3cc4ccccc4cc32)c1 ; COc1c(-c2nc3ccccc3o2)cccc1N(c1cccc(-c2nc3ccccc3o2)c1)c1ccc2c(c1)oc1cc3c(ccc4ccccc43)cc12]

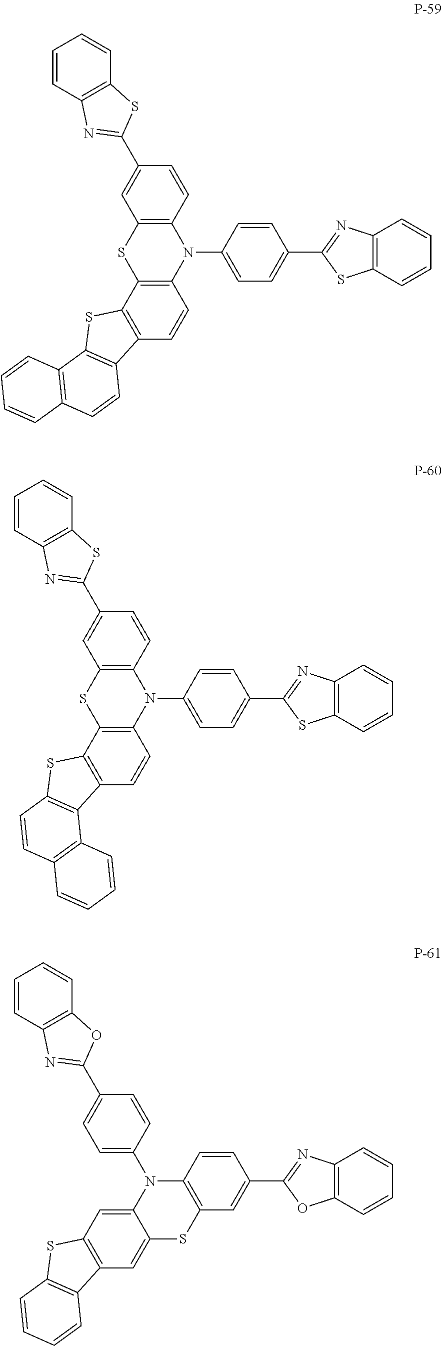 [SMILES: c1ccc2c(c1)ccc1c3ccc4c(c3sc21)Sc1cc(-c2nc3ccccc3s2)ccc1N4c1ccc(-c2nc3ccccc3s2)cc1 ; c1ccc2c(c1)ccc1sc3c4c(ccc3c12)N(c1ccc(-c2nc3ccccc3s2)cc1)c1ccc(-c2nc3ccccc3s2)cc1S4 ; c1ccc2oc(-c3ccc(N4c5ccc(-c6nc7ccccc7o6)cc5Sc5cc6c(cc54)sc4ccccc46)cc3)nc2c1]

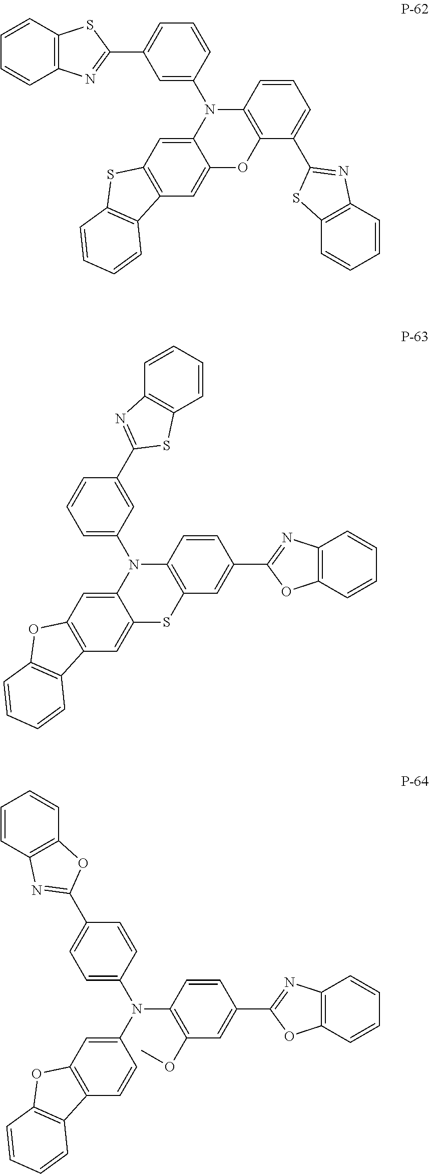 [SMILES: c1cc(-c2nc3ccccc3s2)cc(N2c3cc4sc5ccccc5c4cc3Oc3c(-c4nc5ccccc5s4)cccc32)c1 ; c1cc(-c2nc3ccccc3s2)cc(N2c3ccc(-c4nc5ccccc5o4)cc3Sc3cc4c(cc32)oc2ccccc24)c1 ; COc1cc(-c2nc3ccccc3o2)ccc1N(c1ccc(-c2nc3ccccc3o2)cc1)c1ccc2c(c1)oc1ccccc12]

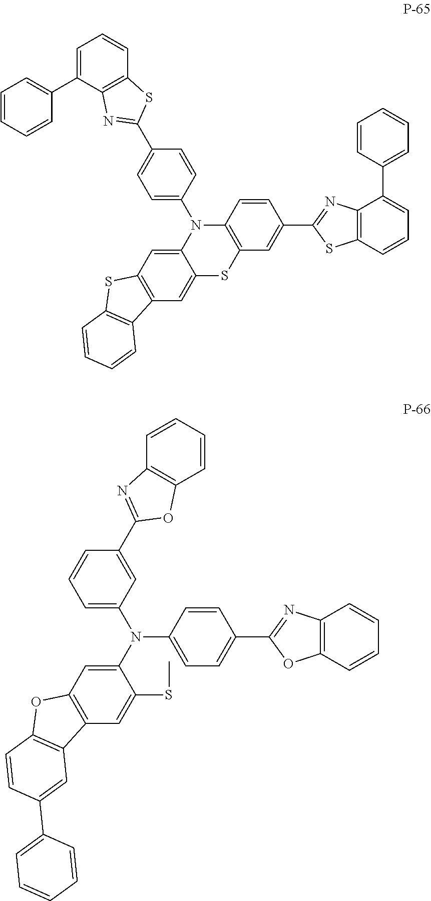 [SMILES: c1ccc(-c2cccc3sc(-c4ccc(N5c6ccc(-c7nc8c(-c9ccccc9)cccc8s7)cc6Sc6cc7c(cc65)sc5ccccc57)cc4)nc23)cc1 ; CSc1cc2c(cc1N(c1ccc(-c3nc4ccccc4o3)cc1)c1cccc(-c3nc4ccccc4o3)c1)oc1ccc(-c3ccccc3)cc12]

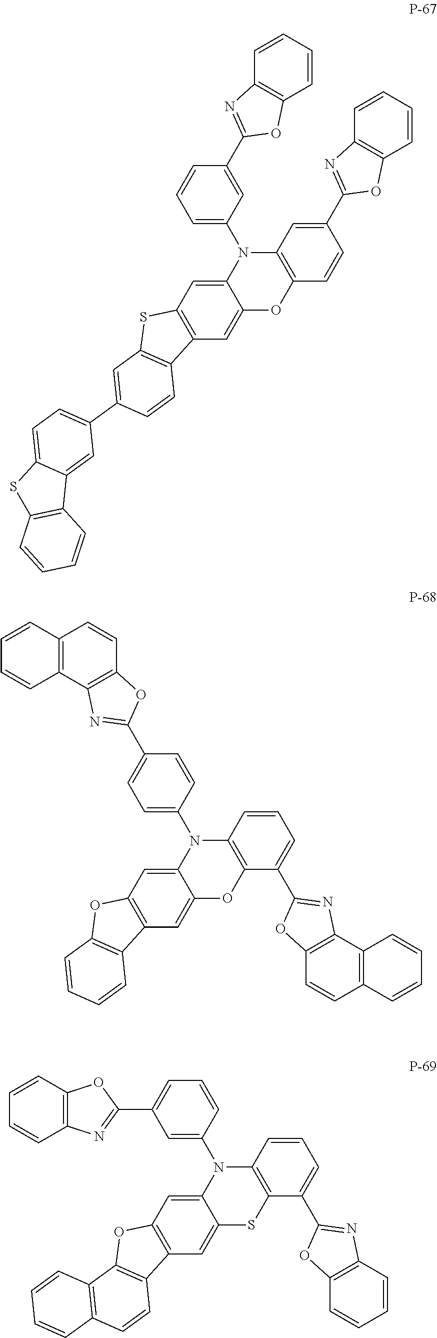 [SMILES: c1cc(-c2nc3ccccc3o2)cc(N2c3cc(-c4nc5ccccc5o4)ccc3Oc3cc4c(cc32)sc2cc(-c3ccc5sc6ccccc6c5c3)ccc24)c1 ; c1cc(-c2nc3c(ccc4ccccc43)o2)c2c(c1)N(c1ccc(-c3nc4c(ccc5ccccc54)o3)cc1)c1cc3oc4ccccc4c3cc1O2 ; c1cc(-c2nc3ccccc3o2)cc(N2c3cc4oc5c6ccccc6ccc5c4cc3Sc3c(-c4nc5ccccc5o4)cccc32)c1]

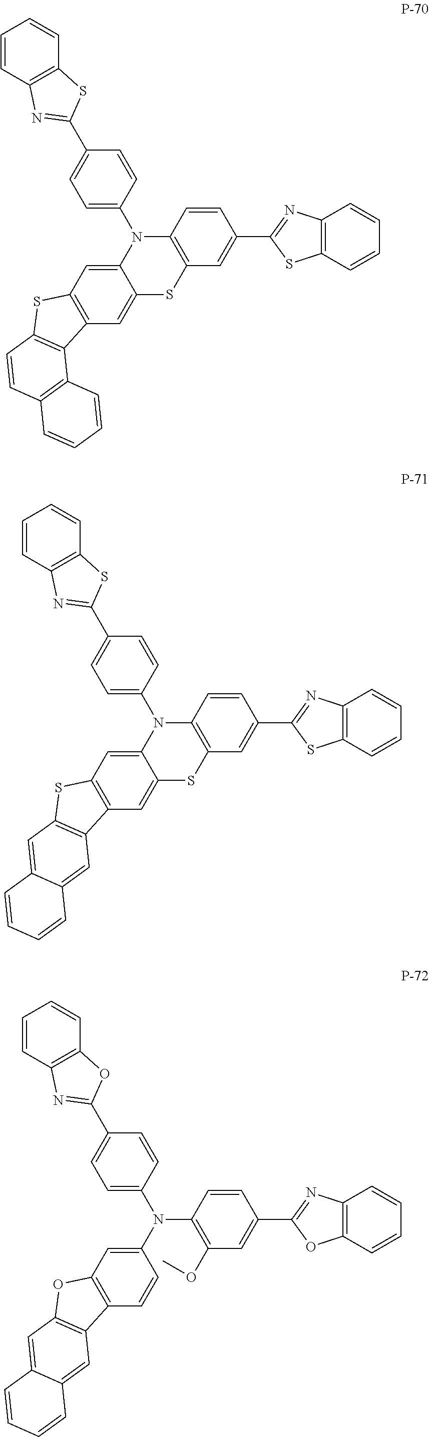 [SMILES: c1ccc2c(c1)ccc1sc3cc4c(cc3c12)Sc1cc(-c2nc3ccccc3s2)ccc1N4c1ccc(-c2nc3ccccc3s2)cc1 ; c1ccc2cc3c(cc2c1)sc1cc2c(cc13)Sc1cc(-c3nc4ccccc4s3)ccc1N2c1ccc(-c2nc3ccccc3s2)cc1 ; COc1cc(-c2nc3ccccc3o2)ccc1N(c1ccc(-c2nc3ccccc3o2)cc1)c1ccc2c(c1)oc1cc3ccccc3cc12]

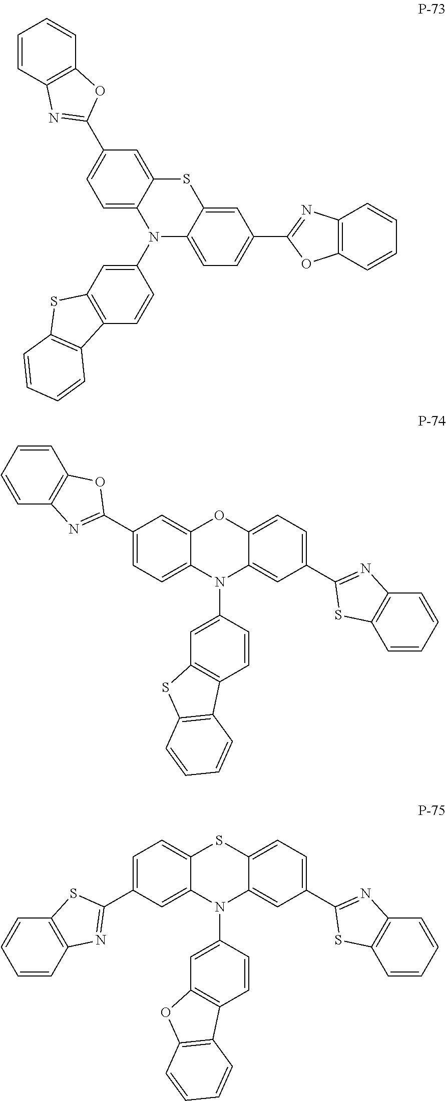

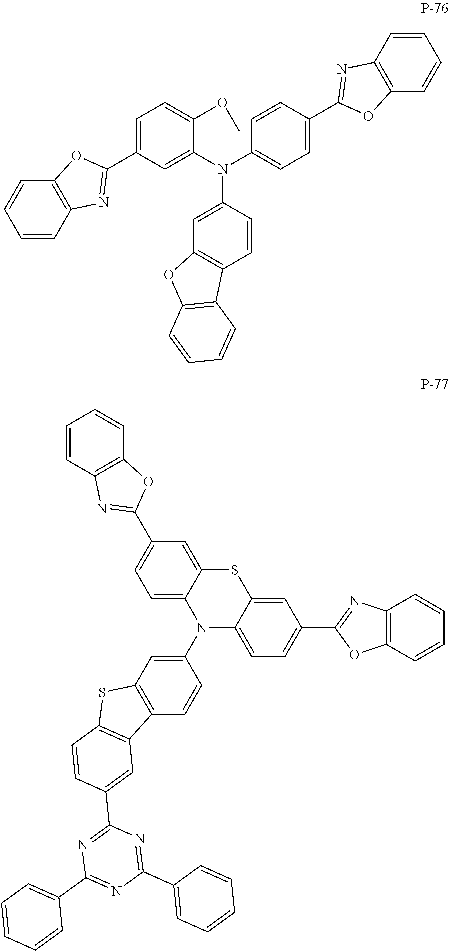 [SMILES: COc1ccc(-c2nc3ccccc3o2)cc1N(c1ccc(-c2nc3ccccc3o2)cc1)c1ccc2c(c1)oc1ccccc12 ; c1ccc(-c2nc(-c3ccccc3)nc(-c3ccc4sc5cc(N6c7ccc(-c8nc9ccccc9o8)cc7Sc7cc(-c8nc9ccccc9o8)ccc76)ccc5c4c3)n2)cc1]

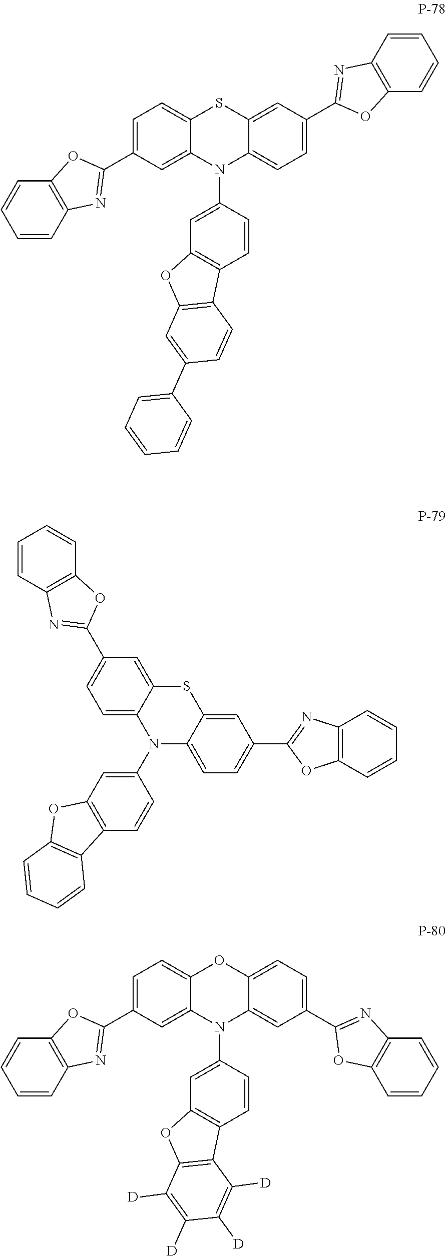 [SMILES: c1ccc(-c2ccc3c(c2)oc2cc(N4c5ccc(-c6nc7ccccc7o6)cc5Sc5ccc(-c6nc7ccccc7o6)cc54)ccc23)cc1 ; c1ccc2oc(-c3ccc4c(c3)Sc3cc(-c5nc6ccccc6o5)ccc3N4c3ccc4c(c3)oc3ccccc34)nc2c1 ; [2H]c1c([2H])c([2H])c2c(oc3cc(N4c5cc(-c6nc7ccccc7o6)ccc5Oc5ccc(-c6nc7ccccc7o6)cc54)ccc32)c1[2H]]

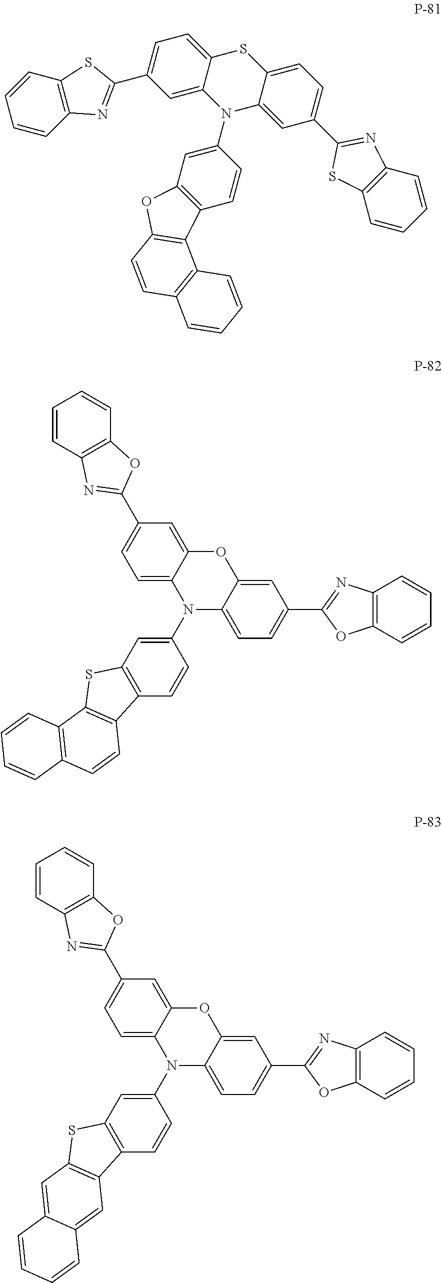 [SMILES: c1ccc2c(c1)ccc1oc3cc(N4c5cc(-c6nc7ccccc7s6)ccc5Sc5ccc(-c6nc7ccccc7s6)cc54)ccc3c12 ; c1ccc2c(c1)ccc1c3ccc(N4c5ccc(-c6nc7ccccc7o6)cc5Oc5cc(-c6nc7ccccc7o6)ccc54)cc3sc21 ; c1ccc2cc3c(cc2c1)sc1cc(N2c4ccc(-c5nc6ccccc6o5)cc4Oc4cc(-c5nc6ccccc6o5)ccc42)ccc13]

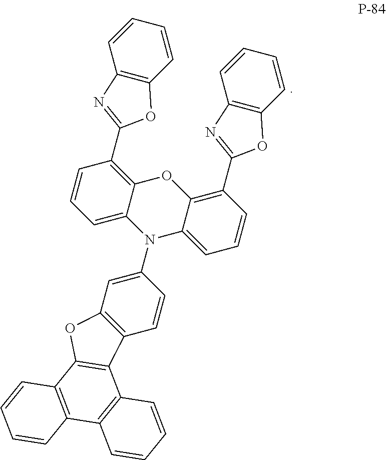 [SMILES: c1cc(-c2nc3ccccc3o2)c2c(c1)N(c1ccc3c(c1)oc1c4ccccc4c4ccccc4c31)c1cccc(-c3nc4ccccc4o3)c1O2]

5. An organic electronic element comprising an anode, a cathode, an organic material layer formed between the anode and the cathode, and a light efficiency enhancing layer, wherein the light efficiency enhancing layer is formed on one side of both surfaces of the anode or the cathode that is not in contact with the organic material layer, wherein the organic material layer or the light efficiency enhancing layer comprises the compound represented by Formula 1 of claim 1.

6. The organic electronic element of claim 5, wherein the organic material layer comprises at least one of a hole injection layer, a hole transport layer, an emitting-auxiliary layer, an emitting layer, an electron transport auxiliary layer, an electron transport layer, and an electron injection layer.

7. The organic electronic element of claim 5, wherein the compound is comprised in the light efficiency enhancing layer.

8. The organic electronic element of claim 5, wherein the organic material layer comprises 2 or more stacks including a hole transport layer, an emitting layer, and an electron transport layer sequentially formed on the anode.

9. The organic electronic element of claim 5, wherein the organic material layer further includes a charge generation layer formed between the 2 or more stacks.

10. An electronic device comprising: a display device including the organic electronic element of claim 5; and a control unit for driving the display device.

11. The electronic device of claim 10, wherein the organic electronic element is any one of an organic electroluminescent device (OLED), an organic solar cell, an organic photoreceptor (OPC), an organic transistor (organic TFT), and an element for monochromic or white illumination.
